(19) United States
(12) Patent Application Publication
(10) Pub. No.: US 2025/0034544 A1
(43) Pub. Date: Jan. 30, 2025

(54) ENGINEERED THREONINE ALDOLASES AND AMINO ACID DECARBOXYLASES

(71) Applicant: Codexis, Inc., Redwood City, CA (US)

(72) Inventors: Oscar Alvizo, Fremont, CA (US); Scott J. Novick, Palo Alto, CA (US); Nandhitha Subramanian, Milton Keynes (GB); Thierry Schlama, Basel (CH)

(21) Appl. No.: 18/714,030

(22) PCT Filed: Dec. 1, 2022
(86) PCT No.: PCT/US2022/080778

§ 371 Date: May 28, 2024

Related U.S. Application Data
(60) Provisional application No. 63/285,377, filed on Dec. 2, 2021.

(51) Int. Cl. C12N 9/88

(52) U.S. Cl. CPC C12N 9/88; C12Y 401/01; C12Y 401/02005

(57) ABSTRACT

The present invention provides engineered threonine aldolase and amino acid decarboxylase polypeptides useful for the production of the chiral tertiary amino alcohols, as well as polynucleotides, compositions, and methods utilizing these engineered polypeptides.

The present application claims priority to U.S. Prov. Pat. Appln. Ser. No. 63/285,377, filed Dec. 2, 2021, which is hereby incorporated by reference in its entirety for all purposes.

### TECHNICAL FIELD

The present invention provides engineered threonine aldolase and amino acid decarboxylase polypeptides useful for the production of chiral tertiary amino alcohols, as well as compositions and methods utilizing these engineered polypeptides.

### REFERENCE TO SEQUENCE LISTING, TABLE OR COMPUTER PROGRAM

The official copy of the Sequence Listing is submitted concurrently with the specification as an XML file, with a file name of “CX2-215WO1_ST26.xml”, a creation date of Nov. 30, 2022, and a size of 729,554 bytes. The Sequence Listing filed is part of the specification and is incorporated in its entirety by reference herein.

### BACKGROUND

Chiral tertiary amino alcohols are important building blocks in the pharmaceutical industry. In particular, chiral tertiary amino alcohols are intermediates for compounds used in the treatment of cystic fibrosis and chronic obstructive pulmonary disease (COPD). However, traditional chemical synthesis methods for chiral tertiary amino alcohols can have drawbacks, such as requiring long synthetic routes, utilizing expensive substrates, providing poor yields and selectivity, and generating toxic by-products.

Therefore, more cost effective and environmentally sensitive biocatalytic synthesis routes are of interest to efficiently produce these compounds. However, only a few biocatalytic routes are known to produce chiral tertiary amino alcohol compounds.

Threonine aldolases (ThrAldos) are a well-characterized class of enzymes prevalent in fungi and bacterial species and useful for the metabolism of threonine, the sole source of carbon and nitrogen for a variety of species (Fesko. Appl Microbiol Biotechnol, 2016, 100:2579-2590). Threonine aldolases natively catalyze the reversible conversion of acetaldehyde and glycine to threonine. Four distinct classes of threonine aldolases are known based on chirality at the α-(L- or D-threonine aldolases) and β-centers of the threonine product. Of these, the D-threonine aldolase is magnesium dependent and is evolutionary and structurally unrelated to the three classes of L-threonine aldolases. Crystal structures of several L-threonine aldolase enzymes have been determined, revealing an active homotetramer with a pyridoxal 5′-phosphate (PLP) cofactor separating the two domains of each monomer (Franz & Stewart. Advances in Applied Microbiology, Volume 88, 2014, 57-101).

Threonine aldolases are highly stereospecific at the α-carbon. Threonine aldolases, together with a second enzyme, have been used to synthesize various compounds, including chiral β-amino alcohols. (Dückers et al. Appl Microbiol Biotechnol, 2010, 88:409-424). In one synthetic route, chiral amino alcohols are synthesized in two steps, the first step using a threonine aldolase to create the intermediate hydroxy-amino acid followed by conversion to the amino alcohol product by a tyrosine decarboxylase (Duckers et al. Appl Microbiol Biotechnol, 2010, 88:409-424). Additional amino acid decarboxylases (together with L-threonine aldolases) may be useful in the synthesis of a range of chiral amino alcohol compounds. In addition to providing a direct synthesis, this route offers several advantages. Namely, the starting materials are inexpensive and readily available. Also, the two enzyme catalytic cascade can be performed under industrial conditions in a one-pot process.

Native and engineered threonine aldolases have been discovered that accept a range of nucleophilic amino acids as substrates. In addition to glycine, threonine aldolases have been discovered that have activity on serine, alanine, and cysteine. A range of electrophilic aldehyde substrates are also known. However, no current reports demonstrate activity of a threonine aldolase on a ketone substrate.

A threonine aldolase with activity on ketones would enable the synthesis of a range useful chiral amino alcohol compounds.

### SUMMARY

The present invention provides novel biocatalysts and associated methods of use for the synthesis chiral β-hydroxy-α-amino acids and tertiary amino alcohols. The threonine aldolase biocatalysts of the present disclosure are engineered polypeptide variants of a homolog gene from Escherichia coli (SEQ ID NO:2) or a homolog gene from Sinorhizobium arboris (SEQ ID NO:158). These engineered polypeptides are capable of catalyzing the conversion of trifluoroacetone and glycine to a β-hydroxy-α-amino acid. The amino acid decarboxylase biocatalysts of the present disclosure are engineered polypeptide variants of the wild-type gene from Planctomycetaceae bacterium (SEQ ID NO: 282). These engineered polypeptides are capable of catalyzing the further conversion of a β-hydroxy-α-amino acid to a tertiary amino alcohol product.

The present invention provides an engineered polypeptide comprising an amino acid sequence with at least 80%, 85%, 90%, 91%, 92%, 93%, 94%, 95%, 96%, 97%, 98%, 99% sequence identity to a reference sequence of SEQ ID NO: 2, comprising at least one substitution or one substitution set at one or more positions selected from 7/8/170/171, 7/170/171/172/195/196, 7/170/171/193, 8, 8/170/194/196, 10, 10/138/205/312, 10/167, 10/169/205, 10/198, 11/81/206, 11/206, 55/60, 55/60/171/174/253, 55/60/173, 55/60/247/250, 55/60/250, 55/60/250/253, 55/60/253, 55/170/253, 57/140/143/206, 60/247/253, 60/250/253, 60/253, 102, 138/167, 138/167/312, 138/169, 138/169/205, 138/169/276, 138/205, 138/245, 140/143/176/206, 140/143/206, 167/205, 169, 170, 170/171, 170/171/172/193/194, 170/172, 170/193/195/196, 170/196, 170/249, 170/253, 171, 171/172, 171/172/193/195/249, 171/174/201/253, 171/195, 171/201/253, 171/249, 171/253, 172, 173/247/250/324, 173/250/253, 174/211/253, 174/253, 191/253, 193, 193/194, 193/195, 194, 194/196, 195, 195/196, 196, 198/201/205, 201/205/246, 205, 206, 245, 249, 250/253, 253, and 276, wherein the positions are numbered with reference to SEQ ID NO: 2. In some additional embodiments, the engineered polypeptide comprises at least one substitution or one substitution set selected from 7E/8L/170V/171A, 7E/170S/171A/193T, 7E/170V/171A/172G/195T/196H, 8L/170S/194T/196H, 8V, 10A, 10A/138C/205E/312P, 10A/167M, 10A/169H/205E, 10A/198T, 11L/81L/206A, 11L/206A, 55S/170L/253W, 55T/60S/253R, 55Y/60S, 55Y/60S/171G/174L/253W, 55Y/60S/173T, 55Y/60S/247L/250G, 55Y/60S/250G, 55Y/60S/250G/253W, 57S/140F/143S/206A, 60S/247L/253W, 60S/250G/253W, 60S/253W, 102H, 138A/167M, 138A/169H/205E, 138A/205E, 138C/167M, 138C/167M/312P, 138C/169H, 138C/169H/276H, 138C/245E, 140F/143S/206A, 140M/143S/176L/206A, 167M/205E, 169H, 170G/171A/172G/193T/194T, 170L/253W, 170S, 170S/172G, 170V/171A, 170V/193T/195T/196H, 170V/196H, 170V/249T, 171A, 171A/172G, 171A/172G/193T/195T/249M, 171A/174L/201C/253R, 171A/195T, 171A/249T, 171A/253W, 171G/201A/253R, 172G, 173T/247L/250G/324T, 173T/250G/253W, 174L/211C/253W, 174L/253W, 191A/253W, 193T, 193T/194T, 193T/195T, 194T, 194T/196H, 195T, 195T/196H, 196H, 198T/201P/205E, 201P/205E/246F, 205E, 206A, 245E, 249T, 250G/253W, 253R, 253W, and 276H, wherein the positions are numbered with reference to SEQ ID NO: 2. In some embodiments, the engineered polypeptide comprises at least one substitution or one substitution set selected from D7E/T8L/I170V/F171A, D7E/I170S/F171A/I193T, D7E/I170V/F171A/N172G/L195T/S196H, T8L/I170S/C194T/S196H, T8V, T10A, T10A/G138C/S205E/H312P, T10A/G167M, T10A/R169H/S205E, T10A/G198T, R11L/A81L/L206A, R11L/L206A, L55S/I170L/H253W, L55T/Q60S/H253R, L55Y/Q60S, L55Y/Q60S/F171G/V174L/H253W, L55Y/Q60S/A173T, L55Y/Q60S/A247L/Q250G, L55Y/Q60S/Q250G, L55Y/Q60S/Q250G/H253W, T57S/N140F/V143S/L206A, Q60S/A247L/H253W, Q60S/Q250G/H253W, Q60S/H253W, P102H, G138A/G167M, G138A/R169H/S205E, G138A/S205E, G138C/G167M, G138C/G167M/H312P, G138C/R169H, G138C/R169H/N276H, G138C/N245E, N140F/V143S/L206A, N140M/V143S/A176L/L206A, G167M/S205E, R169H, 1170G/F171A/N172G/1193T/C194T, 1170L/H253W, 1170S, 1170S/N172G, 1170V/F171A, 1170V/1193T/L195T/S196H, 1170V/S196H, 1170V/L249T, F171A, F171A/N172G, F171A/N172G/1193T/L195T/L249M, F171A/V174L/T201C/H253R, F171A/L195T, F171A/L249T, F171A/H253W, F171G/T201A/H253R, N172G, A173T/A247L/Q250G/A324T, A173T/Q250G/H253W, V174L/R211C/H253W, V174L/H253W, F191A/H253W, 1193T, 1193T/C194T, 1193T/L195T, C194T, C194T/S196H, L195T, L195T/S196H, S196H, G198T/T201P/S205E, T201P/S205E/V246F, S205E, L206A, N245E, L249T, Q250G/H253W, H253R, H253W, and N276H, wherein the positions are numbered with reference to SEQ ID NO: 2. In some further embodiments, the engineered polypeptide comprises an amino acid sequence with at least 80% sequence identity to any even-numbered sequence set forth in SEQ ID NO: 4 to SEQ ID NO: 156.

The present invention also provides an engineered polypeptide comprising an amino acid sequence with at least 80%, 85%, 90%, 91%, 92%, 93%, 94%, 95%, 96%, 97%, 98%, 99% sequence identity to a reference sequence of SEQ ID NO: 158, comprising at least one substitution or one substitution set selected from: 15/268/317, 128, 141/207/210/254/257/258, 141/207/210/254/258, 141/207/255/258, 141/211/212/254, 141/211/257/258, 141/212/254/257/258, 141/212/257/258, 141/254/255/257/258, 141/254/255/258, 141/254/257/258, 141/254/258, 141/255/257/258, 141/257/258, 141/258, 172/174/175, 172/174/175/200/203/205/245, 172/174/175/245, 172/174/175/245/248, 172/174/199/200/203/205/248, 172/174/245, 172/174/248, 172/175, 172/199/203/204/206, 174/175, 174/175/199/200/203/206/245/248, 174/175/199/200/205, 174/175/199/203/204/205, 174/175/245, 174/175/245/248, 174/245/248, 175/176/199, 175/245, 178/181/211/254/255/258, 178/207/211/254/255/257/258, 199/200/203/204/206/245, 199/200/203/206/248, 199/200/204, 199/200/206, 199/203/204, 199/203/204/206/245/248, 203/245/248, 207/210/211/255/257, 207/210/212/257, 207/210/255, 207/211, 207/211/212/254/255/257/258, 207/211/257/258, 207/212/255/258, 210/212/258, 210/254, 211/254/257/258, 211/254/258, 211/257/258, 245/248, 254/255/257/258, 254/255/258, 254/257, and 258, wherein the positions are numbered with reference to SEQ ID NO: 158. In some embodiments, the engineered polypeptide comprises at least one substitution or one substitution set selected from 15L/268Q/317W, 128V, 141S/207L/210R/254M/258Q, 141S/207L/255K/258S, 141S/207M/210R/254M/257Y/258Q, 141S/211G/212G/254M, 141S/211S/257Y/258Q, 141S/212G/254M/257Y/258S, 141S/212G/257Y/258Q, 141S/254M/255K/257Y/258Q, 141S/254M/255K/258Q, 141S/254M/257Y/258Q, 141S/254M/258Q, 141S/255K/257Y/258Q, 141S/257Y/258Q, 141S/258S, 172M/174H/175I/245K/248A, 172M/174H/175V, 172M/174H/175V/200K/203S/205E/245K, 172M/174H/175V/245K, 172M/174H/199T/200K/203S/205E/248A, 172M/174H/245K, 172M/174H/248A, 172M/175V, 172M/199T/203S/204E/206L, 174H/175I/199T/200K/203S/206L/245K/248A, 174H/175I/199T/200K/205E, 174H/175I/245K, 174H/175I/245K/248A, 174H/175V, 174H/175V/199T/203S/204E/205E, 174H/245K/248A, 1751/245K, 175S/176Q/199T, 178G/181C/211S/254M/255K/258Q, 178G/207L/211S/254M/255K/257Y/258S, 199T/200K/203S/204E/206L/245K, 199T/200K/203S/206L/248A, 199T/200K/204E, 199T/200K/206L, 199T/203S/204E, 199T/203S/204E/206L/245K/248A, 203S/245K/248A, 207L/212G/255K/258Q, 207M/210P/211Q/255K/257Y, 207M/210R/212G/257Y, 207M/210R/255K, 207M/211G, 207M/211G/212G/254M/255K/257Y/258Q, 207M/211S, 207M/211S/257Y/258Q, 210R/212G/258Q, 210R/254M, 211A/254M/258S, 211S/254M/257Y/258Q, 211S/257Y/258Q, 245K/248A, 254M/255K/257Y/258Q, 254M/255K/258Q, 254M/257Y, and 258Q, wherein the positions are numbered with reference to SEQ ID NO: 158. In some further embodiments, the engineered polypeptide comprises at least one substitution or one substitution set selected from I15L/L268Q/L317W, N128V, Q141S/C207L/A210R/W254M/A258Q, Q141S/C207L/L255K/A258S, Q141S/C207M/A210R/W254M/L257Y/A258Q, Q141S/E211G/A212G/W254M, Q141S/E211S/L257Y/A258Q, Q141S/A212G/W254M/L257Y/A258S, Q141S/A212G/L257Y/A258Q, Q141S/W254M/L255K/L257Y/A258Q, Q141S/W254M/L255K/A258Q, Q141S/W254M/L257Y/A258Q, Q141S/W254M/A258Q, Q141S/L255K/L257Y/A258Q, Q141S/L257Y/A258Q, Q141S/A258S, G172M/R174H/F175I/F245K/Y248A, G172M/R174H/F175V, G172M/R174H1F175V/F200K/T203S/N205E/F245K, G172M/R174H/F175V/F245K, G172M/R174H1S199T/F200K/T203S/N205E/Y248A, G172M/R174H/F245K, G172M/R174H/Y248A, G172M/F175V, G172M/S199T/T203S/K204E/G206L, R174H/F175I/S199T/F200K/T203S/G206L/F245K/Y248A, R174H/F175I/S199T/F200K/N205E, R174H/F175I/F245K, R174H/F175I/F245K/Y248A, R174H/F175V, R174H/F175V/S199T/T203S/K204E/N205E, R174H/F245K/Y248A, F175I/F245K, F175S/A176Q/S199T, A178G/S181C/E211S/W254M/L255K/A258Q, A178G/C207L/E211S/W254M/L255K/L257Y/A258S, S199T/F200K/T203S/K204E/G206L/F245K, S199T/F200K/T203S/G206L/Y248A, S199T/F200K/K204E, S199T/F200K/G206L, S199T/T203S/K204E, S199T/T203S/K204E/G206L/F245K/Y248A, T203S/F245K/Y248A, C207L/A212G/L255K/A258Q, C207M/A210P/E211Q/L255K/L257Y, C207M/A210R/A212G/L257Y, C207M/A210R/L255K, C207M/E211G, C207M/E211G/A212G/W254M/L255K/L257Y/A258Q, C207M/E211S, C207M/E211S/L257Y/A258Q, A210R/A212G/A258Q, A210R/W254M, E211A/W254M/A258S, E211S/W254M/L257Y/A258Q, E211S/L257Y/A258Q, F245K/Y248A, W254M/L255K/L257Y/A258Q, W254M/L255K/A258Q, W254M/L257Y, and A258Q, wherein the positions are numbered with reference to SEQ ID NO: 158. In some further embodiments, the engineered polypeptide comprises an amino acid sequence with at least 80% sequence identity to any even-numbered sequence set forth in SEQ ID NO: 160 to SEQ ID NO: 280.

The present invention further provides an engineered polypeptide comprising an amino acid sequence with at least 80%, 85%, 90%, 91%, 92%, 93%, 94%, 95%, 96%, 97%, 98%, 99% sequence identity to a reference sequence of SEQ ID NO: 282, comprising at least at least one substitution or one substitution set selected from 48, 49, 55/74/110/121/194/233/253, 55/74/110/194/233/281/324, 55/74/110/211, 55/74/121/233/253/324, 55/74/194/233, 55/194/211/233/253, 55/211/253/324, 56, 63/398, 66/86/198/235/329, 66/198/202/290/316/329, 66/202/290/329, 66/290/329, 72, 74/110/121/194, 74/110/233/324, 74/110/324, 74/121/194/233/253, 74/121/194/233/324, 74/194, 74/194/233, 74/194/253, 80, 86, 86/162/186/187/202/203/235/329, 103, 135, 162, 162/187/202/235/252/290/316, 174, 183, 198, 245, 248, 258, 279, 280, and 350, wherein the positions are numbered with reference to SEQ ID NO: 282.

In some additional embodiments, the engineered polypeptide comprises at least one substitution or one substitution set selected from 48V, 49N, 55I/74F/110Y/121L/194L/233I/253K, 55I/74F/110Y/194L/233I/281H/324W, 55I/74F/110Y/211S, 55I/74F/121L/233I/253K/324W, 55I/74F/194L/233I, 55I/194L/211S/233I/253K, 55I/211S/253K/324W, 56D, 63T/398L, 66N/86F/198G/235V/329S, 66N/198G/202Y/290I/316L/329S, 66N/202Y/290I/329S, 66N/290I/3295, 72A, 72E, 74F/110Y/121L/194L, 74F/110Y/2331/324W, 74F/110Y/324W, 74F/121L/194L/233I/253K, 74F/121L/194L/233I/324W, 74F/194L, 74F/194L/233I, 74F/194L/253K, 80K, 86F/162I/186D/187I/202Y/203Y/235V/329S, 86T, 103G, 135I, 162I/187I/202Y/235V/252A/290I/316L, 162L, 174C, 183V, 198S, 245E, 245W, 248S, 258S, 279P, 280A, and 350E, wherein the positions are numbered with reference to SEQ ID NO: 282. In some further embodiments, the engineered polypeptide comprises at least at least one substitution or one substitution set selected from L48V, P49N, V55I/I74F/F110Y/A121L/F194L/L233I/R253K, V55I/I74F/F110Y/F194L/L233I/I281H/R324W, V55I/I74F/F110Y/A211S, V55I/I74F/A121L/L233I/R253K/R324W, V55I/174F/F194L/L233I, V55I/F194L/A211S/L233I/R253K, V55I/A211S/R253K/R324W, N56D, A63T/P398L, S66N/M86F/A198G/I235V/A329S, S66N/A198G/S202Y/V290I/F316L/A329S, S66N/S202Y/V290I/A329S, S66N/V290I/A329S, R72A, R72E, I74F/F110Y/A121L/F194L, 174F/F110Y/L233I/R324W, 174F/F110Y/R324W, I74F/A121L/F194L/L233I/R253K, I74F/A121L/F194L/L233I/R324W, I74F/F194L, I74F/F194L/L233I, I74F/F194L/R253K, A80K, M86F/M162I/N186D/V187I/S202Y/H203Y/I235V/A329S, M86T, E103G, L135I, M162I/V187I/S202Y/1235V/T252A/V290I/F316L, M162L, A174C, G183V, A198S, I245E, I245W, G248S, E258S, N279P, G280A, and N350E, wherein the positions are numbered with reference to SEQ ID NO: 282. In some additional embodiments, the engineered polypeptide comprises an amino acid sequence with at least 80% sequence identity to any even-numbered sequence set forth in SEQ ID NO:284 to SEQ ID NO: 366.

The present invention also provides an engineered polynucleotide encoding at least one engineered polypeptide described in the above paragraphs. In some embodiments, the engineered polynucleotide comprises the odd-numbered sequences set forth in SEQ ID NO: 5 to SEQ ID NO: 365.

The present invention further provides vectors comprising at least one engineered polynucleotide described above. In some embodiments, the vectors further comprise at least one control sequence.

The present invention also provides host cells comprising the vectors provided herein. In some embodiments, the host cell produces at least one engineered polypeptide provided herein.

The present invention further provides methods of producing an engineered threonine aldolase or amino acid decarboxylase polypeptide, comprising the steps of culturing the host cell provided herein under conditions such that the engineered polynucleotide is expressed and the engineered polypeptide is produced. In some embodiments, the methods further comprise the step of recovering the engineered polypeptide.

### DESCRIPTION OF THE INVENTION

Unless defined otherwise, all technical and scientific terms used herein generally have the same meaning as commonly understood by one of ordinary skill in the art to which this invention pertains. Generally, the nomenclature used herein and the laboratory procedures of cell culture, molecular genetics, microbiology, organic chemistry, analytical chemistry and nucleic acid chemistry described below are those well-known and commonly employed in the art. Such techniques are well-known and described in numerous texts and reference works well known to those of skill in the art. Standard techniques, or modifications thereof, are used for chemical syntheses and chemical analyses. All patents, patent applications, articles and publications mentioned herein, both supra and infra, are hereby expressly incorporated herein by reference.

Although any suitable methods and materials similar or equivalent to those described herein find use in the practice of the present invention, some methods and materials are described herein. It is to be understood that this invention is not limited to the particular methodology, protocols, and reagents described, as these may vary, depending upon the context they are used by those of skill in the art. Accordingly, the terms defined immediately below are more fully described by reference to the invention as a whole.

It is to be understood that both the foregoing general description and the following detailed description are exemplary and explanatory only and are not restrictive of the present invention. The section headings used herein are for organizational purposes only and not to be construed as limiting the subject matter described. Numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used in this specification and the appended claims, the singular forms “a”, “an” and “the” include plural referents unless the context clearly indicates otherwise. Thus, for example, reference to “a polypeptide” includes more than one polypeptide. Similarly, “comprise,” “comprises,” “comprising” “include,” “includes,” and “including” are interchangeable and not intended to be limiting.

It is to be understood that where descriptions of various embodiments use the term “comprising,” those skilled in the art would understand that in some specific instances, an embodiment can be alternatively described using language “consisting essentially of” or “consisting of” It is to be further understood that where descriptions of various embodiments use the term “optional” or “optionally” the subsequently described event or circumstance may or may not occur, and that the description includes instances where the event or circumstance occurs and instances in which it does not. It is to be understood that both the foregoing general description, and the following detailed description are exemplary and explanatory only and are not restrictive of this disclosure. The section headings used herein are for organizational purposes only and not to be construed as limiting the subject matter described.

### Abbreviations:

The abbreviations used for the genetically encoded amino acids are conventional and are as follows:

| Amino Acid | Three-Letter | One-Letter Abbreviation |
|---|---|---|
| Alanine | Ala | A |
| Arginine | Arg | R |
| Asparagine | Asn | N |
| Aspartate | Asp | D |
| Cysteine | Cys | C |
| Glutamate | Glu | E |
| Glutamine | Gln | Q |
| Glycine | Gly | G |
| Histidine | HIS | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

When the three-letter abbreviations are used, unless specifically preceded by an “L” or a “D” or clear from the context in which the abbreviation is used, the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively.

When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

The abbreviations used for the genetically encoding nucleosides are conventional and are as follows: adenosine (A); guanosine (G); cytidine (C); thymidine (T); and uridine (U). Unless specifically delineated, the abbreviated nucleotides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

### Definitions

In reference to the present invention, the technical and scientific terms used in the descriptions herein will have the meanings commonly understood by one of ordinary skill in the art, unless specifically defined otherwise. Accordingly, the following terms are intended to have the following meanings.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Protein,” “polypeptide,” and “peptide” are used interchangeably herein to denote a polymer of at least two amino acids covalently linked by an amide bond, regardless of length or post-translational modification (e.g., glycosylation, phosphorylation, lipidation, myristilation, ubiquitination, etc.). Included within this definition are D- and L-amino acids, and mixtures of D- and L-amino acids, as well as polymers comprising D- and L-amino acids, and mixtures of D- and L-amino acids.

“Amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes.

As used herein, “polynucleotide” and “nucleic acid’ refer to two or more nucleosides that are covalently linked together. The polynucleotide may be wholly comprised of ribonucleotides (i.e., RNA), wholly comprised of 2′ deoxyribonucleotides (i.e., DNA), or comprised of mixtures of ribo- and 2′ deoxyribonucleotides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. In some embodiments, such modified or synthetic nucleobases are nucleobases encoding amino acid sequences.

“Threonine aldolase” or “ThrAldo,” as used herein, refers to a wild-type or engineered enzyme having threonine aldolase activity (EC 4.1.2.5), natively catalyzing the PLP-dependent reversible conversion of acetaldehyde and glycine to threonine. Threonine aldolases also catalyze other reactions, and the definition of threonine aldolase activity is intended to be non-limiting with regard to substrates and products. While the threonine aldolase enzymes of the present invention are derived from Escherichia coli or Sinorhizobium arboris, the present invention is not thus limited, and threonine aldolase enzymes may be derived from any suitable organism or created synthetically. Threonine aldolases, as used herein, include naturally occurring (wild-type) threonine aldolases as well as non-naturally occurring engineered polypeptides generated by human manipulation.

“Amino acid decarboxylase” or “AADC” refers to a PLP-dependent enzyme categorized under EC 4.1.1 having activity to decarboxylate or remove a carboxyl group from an amino acid, amino acid analog, or related molecule. While the AADC enzymes of the present invention are derived from Planctomycetaceae bacterium, the present invention is not thus limited, and AADC enzymes may be derived from any suitable organism or created synthetically. Amino acid decarboxylases, as used herein, include naturally occurring (wild-type) amino acid decarboxylases, as well as non-naturally occurring engineered polypeptides generated by human manipulation.

“Coding sequence” refers to that portion of a nucleic acid (e.g., a gene) that encodes an amino acid sequence of a protein.

“Naturally-occurring” or “wild-type” refers to the form found in nature. For example, a naturally occurring or wild-type polypeptide or polynucleotide sequence is a sequence present in an organism that can be isolated from a source in nature and which has not been intentionally modified by human manipulation.

As used herein, “recombinant,” “engineered,” and “non-naturally occurring” when used with reference to a cell, nucleic acid, or polypeptide, refer to a material, or a material corresponding to the natural or native form of the material, that has been modified in a manner that would not otherwise exist in nature. In some embodiments, the cell, nucleic acid or polypeptide is identical a naturally occurring cell, nucleic acid or polypeptide, but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percentage of sequence identity” and “percentage homology” are used interchangeably herein to refer to comparisons among polynucleotides or polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 2:482 [1981]), by the homology alignment algorithm of Needleman and Wunsch (Needleman and Wunsch, J. Mol. Biol., 48:443 [1970]), by the search for similarity method of Pearson and Lipman (Pearson and Lipman, Proc. Natl. Acad. Sci. USA 85:2444 [1988]), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms, which are described by Altschul et al. (See, Altschul et al., J. Mol. Biol., 215: 403-410 [1990]; and Altschul et al., Nucl. Acids Res., 3389-3402 [1977], respectively). Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (See, Henikoff and Henikoff, Proc. Natl. Acad. Sci. USA 89:10915 [1989]). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, a “reference sequence based on SEQ ID NO:4 having at the residue corresponding to X14 a valine” or X14V refers to a reference sequence in which the corresponding residue at X14 in SEQ ID NO:4, which is a tyrosine, has been changed to valine.

“Comparison window” refers to a conceptual segment of at least about 20 contiguous nucleotide positions or amino acids residues wherein a sequence may be compared to a reference sequence of at least 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. The comparison window can be longer than 20 contiguous residues, and includes, optionally 30, 40, 50, 100, or longer windows.

As used herein, “substantial identity” refers to a polynucleotide or polypeptide sequence that has at least 80 percent sequence identity, at least 85 percent identity, at least between 89 to 95 percent sequence identity, or more usually, at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In some specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, at least 95 percent sequence identity or more (e.g., 99 percent sequence identity). In some embodiments, residue positions that are not identical in sequences being compared differ by conservative amino acid substitutions.

“Corresponding to,” “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered threonine aldolase or amino acid decarboxylase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Amino acid difference” or “residue difference” refers to a change in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X25 as compared to SEQ ID NO: 2” refers to a change of the amino acid residue at the polypeptide position corresponding to position 25 of SEQ ID NO:2. Thus, if the reference polypeptide of SEQ ID NO: 2 has a valine at position 25, then a “residue difference at position X25 as compared to SEQ ID NO:2” an amino acid substitution of any residue other than valine at the position of the polypeptide corresponding to position 25 of SEQ ID NO: 2. In most instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some embodiments, more than one amino acid can appear in a specified residue position (i.e., the alternative amino acids can be listed in the form XnY/Z, where Y and Z represent alternate amino acid residues). In some instances (e.g., in Tables 5.1, 6.1, 7.1, 8.1, 9.1, 10.1, 11.1 and 12.1) the present invention also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. Furthermore, in some instances, a polypeptide of the present invention can include one or more amino acid residue differences relative to a reference sequence, which is indicated by a list of the specified positions where changes are made relative to the reference sequence. In some additional embodiments, the present invention provides engineered polypeptide sequences comprising both conservative and non-conservative amino acid substitutions.

As used herein, “conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain is substituted with another aliphatic amino acid (e.g., alanine, valine, leucine, and isoleucine); an amino acid with an hydroxyl side chain is substituted with another amino acid with a hydroxyl side chain (e.g., serine and threonine); an amino acid having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively. Exemplary conservative substitutions are provided in Table 1 below.

TABLE 1
Conservative Amino Acid Substitution Examples

| Residue | Possible Conservative Substitutions |
|---|---|
| A, L, V, I | Other aliphatic (A, L, V, I) |
| | Other non-polar (A, L, V, I, G, M) |
| G, M | Other non-polar (A, L, V, I, G, M) |
| D, E | Other acidic (D, E) |
| K, R | Other basic (K, R) |
| N, Q, S, T | Other polar |
| H, Y, W, F | Other aromatic (H, Y, W, F) |
| C, P | None |

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine), (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered threonine aldolase or amino acid decarboxylase enzyme. Deletions can be directed to the internal portions and/or terminal portions of the polypeptide. In various embodiments, the deletion can comprise a continuous segment or can be discontinuous.

“Insertion” refers to modification to the polypeptide by addition of one or more amino acids from the reference polypeptide. In some embodiments, the improved engineered threonine aldolase or amino acid decarboxylase enzymes comprise insertions of one or more amino acids to the naturally occurring threonine aldolase or amino acid decarboxylase polypeptide as well as insertions of one or more amino acids to other improved threonine aldolase or amino acid decarboxylase polypeptides. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Fragment” as used herein refers to a polypeptide that has an amino-terminal and/or carboxy-terminal deletion, but where the remaining amino acid sequence is identical to the corresponding positions in the sequence. Fragments can be at least 14 amino acids long, at least 20 amino acids long, at least 50 amino acids long or longer, and up to 70%, 80%, 90%, 95%, 98%, and 99% of the full-length threonine aldolase or amino acid decarboxylase polypeptide, for example the polypeptide of SEQ ID NO: 4 or an engineered threonine aldolase or amino acid decarboxylase provided in the even-numbered sequences of SEQ ID NO: 4-156, 160-280, and 284-366.

“Isolated polypeptide” refers to a polypeptide which is substantially separated from other contaminants that naturally accompany it, e.g., protein, lipids, and polynucleotides. The term embraces polypeptides which have been removed or purified from their naturally-occurring environment or expression system (e.g., host cell or in vitro synthesis). The engineered threonine aldolase or amino acid decarboxylase enzymes may be present within a cell, present in the cellular medium, or prepared in various forms, such as lysates or isolated preparations. As such, in some embodiments, the engineered threonine aldolase or amino acid decarboxylase enzyme can be an isolated polypeptide.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure threonine aldolase or amino acid decarboxylase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated engineered threonine aldolase or amino acid decarboxylase polypeptide is a substantially pure polypeptide composition.

“Stereoselective” refers to a preference for formation of one stereoisomer over another in a chemical or enzymatic reaction. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (e.e.) calculated therefrom according to the formula [major enantiomer−minor enantiomer]/[major enantiomer+minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (d.e.). Enantiomeric excess and diastereomeric excess are types of stereomeric excess.

“Highly stereoselective” refers to a chemical or enzymatic reaction that is capable of converting a substrate or substrates (e.g., substrate compounds (2) and (3)), to the corresponding amine product (e.g., Compound (1)), with at least about 85% stereomeric excess.

As used herein, “improved enzyme property” refers to at least one improved property of an enzyme. In some embodiments, the present invention provides engineered threonine aldolase or amino acid decarboxylase polypeptides that exhibit an improvement in any enzyme property as compared to a reference threonine aldolase or amino acid decarboxylase polypeptide and/or a wild-type threonine aldolase or amino acid decarboxylase polypeptide, and/or another engineered threonine aldolase or amino acid decarboxylase polypeptide. For the engineered threonine aldolase or amino acid decarboxylase polypeptides described herein, the comparison is generally made to the parent enzyme from which the threonine aldolase or amino acid decarboxylase is derived, although in some embodiments, the reference enzyme can be another improved engineered threonine aldolase or amino acid decarboxylase. Thus, the level of “improvement” can be determined and compared between various threonine aldolase or amino acid decarboxylase polypeptides, including wild-type, as well as engineered threonine aldolases or amino acid decarboxylases. Improved properties include, but are not limited, to such properties as enzymatic activity (which can be expressed in terms of percent conversion of the substrate), thermo stability, solvent stability, pH activity profile, cofactor requirements, refractoriness to inhibitors (e.g., substrate or product inhibition), stereospecificity, and/or stereoselectivity (including enantioselectivity).

“Increased enzymatic activity” refers to an improved property of the engineered threonine aldolase or amino acid decarboxylase polypeptides, which can be represented by an increase in specific activity (e.g., product produced/time/weight protein) or an increase in percent conversion of the substrate to the product (e.g., percent conversion of starting amount of substrate to product in a specified time period using a specified amount of threonine aldolase or amino acid decarboxylase) as compared to the reference threonine aldolase or amino acid decarboxylase enzyme. Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 1.2 times the enzymatic activity of the corresponding parent enzyme, to as much as 2 times, 5 times, 10 times, 20 times, 25 times, 50 times or more enzymatic activity than the naturally occurring or another engineered threonine aldolase or amino acid decarboxylase from which the threonine aldolase or amino acid decarboxylase polypeptides were derived. Threonine aldolase or amino acid decarboxylase activity can be measured by any one of standard assays, such as by monitoring changes in properties of substrates, cofactors, or products. In some embodiments, the amount of products generated can be measured by Liquid Chromatography-Mass Spectrometry (LC-MS). Comparisons of enzyme activities are made using a defined preparation of enzyme, a defined assay under a set condition, and one or more defined substrates, as further described in detail herein. Generally, when lysates are compared, the numbers of cells and the amount of protein assayed are determined as well as use of identical expression systems and identical host cells to minimize variations in amount of enzyme produced by the host cells and present in the lysates.

“Conversion” refers to the enzymatic conversion of the substrate(s) to the corresponding product(s). “Percent conversion” refers to the percent of the substrate that is converted to the product within a period of time under specified conditions. Thus, the “enzymatic activity” or “activity” of a threonine aldolase or amino acid decarboxylase polypeptide can be expressed as “percent conversion” of the substrate to the product.

“Thermostable” refers to a threonine aldolase or amino acid decarboxylase polypeptide that maintains similar activity (more than 60% to 80% for example) after exposure to elevated temperatures (e.g., 40-80° C.) for a period of time (e.g., 0.5-24 hrs) compared to the parent enzyme exposed to the same elevated temperature.

“Solvent stable” refers to a threonine aldolase or amino acid decarboxylase polypeptide that maintains similar activity (more than e.g., 60% to 80%) after exposure to varying concentrations (e.g., 5-99%) of solvent (ethanol, isopropyl alcohol, dimethylsulfoxide (DMSO), tetrahydrofuran, 2-methyltetrahydrofuran, acetone, toluene, butyl acetate, methyl tert-butyl ether, etc.) for a period of time (e.g., 0.5-24 hrs) compared to the parent enzyme exposed to the same concentration of the same solvent.

“Thermo- and solvent stable” refers to a threonine aldolase or amino acid decarboxylase polypeptide that is both thermostable and solvent stable.

The term “stringent hybridization conditions” is used herein to refer to conditions under which nucleic acid hybrids are stable. As known to those of skill in the art, the stability of hybrids is reflected in the melting temperature (Tₘ) of the hybrids. In general, the stability of a hybrid is a function of ion strength, temperature, G/C content, and the presence of chaotropic agents. The Tₘ values for polynucleotides can be calculated using known methods for predicting melting temperatures (See e.g., Baldino et al., Meth. Enzymol., 168:761-777 [1989]; Bolton et al., Proc. Natl. Acad. Sci. USA 48:1390 [1962]; Bresslauer et al., Proc. Natl. Acad. Sci. USA 83:8893-8897 [1986]; Freier et al., Proc. Natl. Acad. Sci. USA 83:9373-9377 [1986]; Kierzek et al., Biochem., 25:7840-7846 [1986]; Rychlik et al., 1990, Nucl. Acids Res., 18:6409-6412 [1990] (erratum, Nucl. Acids Res., 19:698 [1991]); Sambrook et al., supra); Suggs et al., 1981, in Developmental Biology Using Purified Genes, Brown et al. [eds.], pp. 683-693, Academic Press, Cambridge, MA [1981]; and Wetmur, Crit. Rev. Biochem. Mol. Biol., 26:227-259 [1991]). In some embodiments, the polynucleotide encodes the polypeptide disclosed herein and hybridizes under defined conditions, such as moderately stringent or highly stringent conditions, to the complement of a sequence encoding an engineered threonine aldolase or amino acid decarboxylase enzyme of the present invention.

“Hybridization stringency” relates to hybridization conditions, such as washing conditions, in the hybridization of nucleic acids. Generally, hybridization reactions are performed under conditions of lower stringency, followed by washes of varying but higher stringency. The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tₘ as determined under the solution condition for a defined polynucleotide sequence. In some embodiments, a high stringency condition refers to conditions that permit hybridization of only those nucleic acid sequences that form stable hybrids in 0.018 M NaCl at 65° C. (i.e., if a hybrid is not stable in 0.018 M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

“Heterologous” polynucleotide refers to any polynucleotide that is introduced into a host cell by laboratory techniques, and includes polynucleotides that are removed from a host cell, subjected to laboratory manipulation, and then reintroduced into a host cell.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the threonine aldolase or amino acid decarboxylase enzymes may be codon optimized for optimal production from the host organism selected for expression.

As used herein, “preferred, optimal, high codon usage bias codons” refers interchangeably to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (See e.g., GCG CodonPreference, Genetics Computer Group Wisconsin Package; CodonW, Peden, University of Nottingham; McInerney, Bioinform., 14:372-73 [1998]; Stenico et al., Nucl. Acids Res., 222437-46 [1994]; Wright, Gene 87:23-29 [1990]). Codon usage tables are available for many different organisms (See e.g., Wada et al., Nucl. Acids Res., 20:2111-2118 [1992]; Nakamura et al., Nucl. Acids Res., 28:292 [2000]; Duret, et al., supra; Henaut and Danchin, in Escherichia coli and Salmonella, Neidhardt, et al. (eds.), ASM Press, Washington D.C., p. 2047-2066 [1996]). The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (See e.g., Mount, Bioinformatics: Sequence and Genome Analysis, Chapter 8, Cold Spring Harbor Laboratory Press, Cold Spring Harbor, N.Y. [2001]; Uberbacher, Meth. Enzymol., 266:259-281 [1996]; and Tiwari et al., Comput. Appl. Biosci., 13:263-270 [1997]).

“Control sequence” is defined herein to include all components, which are necessary or advantageous for the expression of a polynucleotide and/or polypeptide of the present invention. Each control sequence may be native or foreign to the nucleic acid sequence encoding the polypeptide. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the nucleic acid sequence encoding a polypeptide.

“Operably linked” is defined herein as a configuration in which a control sequence is appropriately placed (i.e., in a functional relationship) at a position relative to a polynucleotide of interest such that the control sequence directs or regulates the expression of the polynucleotide and/or polypeptide of interest.

“Promoter sequence” refers to a nucleic acid sequence that is recognized by a host cell for expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Suitable reaction conditions” refer to those conditions in the biocatalytic reaction solution (e.g., ranges of enzyme loading, substrate loading, cofactor loading, temperature, pH, buffers, co-solvents, etc.) under which threonine aldolase or amino acid decarboxylase polypeptide of the present invention is capable of converting one or more substrate compounds to a product compound (e.g., conversion of compound (1) and compound (2) to compound (3) or conversion of compound (3) to compound (4), as shown in Scheme 1). Exemplary “suitable reaction conditions” are provided in the present invention and illustrated by the Examples.

“Composition” refers to a mixture or combination of one or more substances, wherein each substance or component of the composition retains its individual properties. As used herein, a biocatalytic composition refers to a combination of one or more substances useful for biocatalysis.

“Loading”, such as in “compound loading” or “enzyme loading” or “cofactor loading” refers to the concentration or amount of a component in a reaction mixture at the start of the reaction.

“Substrate” in the context of a biocatalyst mediated process refers to the compound or molecule acted on by the biocatalyst. For example, compound (1) and compound (2) are substrates for a threonine aldolase, while compound (3) is a substrate for an amino acid decarboxylase.

“Product” in the context of a biocatalyst mediated process refers to the compound or molecule resulting from the action of the biocatalyst. For example, compound (3) is a product of the threonine aldolase-mediated conversion of compound (1) and compound (2) Similarly, compound (4) is a product for the amino acid decarboxylase-mediated conversion of compound (3).

“Alkyl” refers to saturated hydrocarbon groups of from 1 to 18 carbon atoms inclusively, either straight chained or branched, more preferably from 1 to 8 carbon atoms inclusively, and most preferably 1 to 6 carbon atoms inclusively. An alkyl with a specified number of carbon atoms is denoted in parenthesis (e.g., (C₁-C₆)alkyl refers to an alkyl of 1 to 6 carbon atoms).

“Alkenyl” refers to hydrocarbon groups of from 2 to 12 carbon atoms inclusively, either straight or branched containing at least one double bond but optionally containing more than one double bond.

“Alkynyl” refers to hydrocarbon groups of from 2 to 12 carbon atoms inclusively, either straight or branched containing at least one triple bond but optionally containing more than one triple bond, and additionally optionally containing one or more double bonded moieties.

“Alkylene” refers to a straight or branched chain divalent hydrocarbon radical having from 1 to 18 carbon atoms inclusively, more preferably from 1 to 8 carbon atoms inclusively, and most preferably 1 to 6 carbon atoms inclusively, optionally substituted with one or more suitable substituents. Exemplary “alkylenes” include, but are not limited to, methylene, ethylene, propylene, butylene, and the like.

“Alkenylene” refers to a straight or branched chain divalent hydrocarbon radical having 2 to 12 carbon atoms inclusively and one or more carbon-carbon double bonds, more preferably from 2 to 8 carbon atoms inclusively, and most preferably 2 to 6 carbon atoms inclusively, optionally substituted with one or more suitable substituents.

“Heteroalkyl, “heteroalkenyl,” and heteroalkynyl,” refer respectively, to alkyl, alkenyl and alkynyl as defined herein in which one or more of the carbon atoms are each independently replaced with the same or different heteroatoms or heteroatomic groups. Heteroatoms and/or heteroatomic groups which can replace the carbon atoms include, but are not limited to, —O—, —S—, —S—O—, —NR^{γ}—, —PH—, —S(O)—, —S(O)₂—, —S(O)NR^{γ}—, —S(O)₂NR^{γ}—, and the like, including combinations thereof, where each R^{γ} is independently selected from hydrogen, alkyl, cycloalkyl, heterocycloalkyl, aryl, and heteroaryl.

“Aryl” refers to an unsaturated aromatic carbocyclic group of from 6 to 12 carbon atoms inclusively having a single ring (e.g., phenyl) or multiple condensed rings (e.g., naphthyl or anthryl). Exemplary aryls include phenyl, pyridyl, naphthyl and the like.

“Arylalkyl” refers to an alkyl substituted with an aryl (i.e., aryl-alkyl-groups), preferably having from 1 to 6 carbon atoms inclusively in the alkyl moiety and from 6 to 12 carbon atoms inclusively in the aryl moiety. Such arylalkyl groups are exemplified by benzyl, phenethyl and the like.

“Aryloxy” refers to —OR^{λ} groups, where R^{λ} is an aryl group, which can be optionally substituted.

“Cycloalkyl” refers to cyclic alkyl groups of from 3 to 12 carbon atoms inclusively having a single cyclic ring or multiple condensed rings which can be optionally substituted with from 1 to 3 alkyl groups. Exemplary cycloalkyl groups include, but are not limited to, single ring structures such as cyclopropyl, cyclobutyl, cyclopentyl, cyclooctyl, 1-methylcyclopropyl, 2-methylcyclopentyl, 2-methylcyclooctyl, and the like, or multiple ring structures, including bridged ring systems, such as adamantyl, and the like.

“Cycloalkylalkyl” refers to an alkyl substituted with a cycloalkyl (i.e., cycloalkyl-alkyl-groups), preferably having from 1 to 6 carbon atoms inclusively in the alkyl moiety and from 3 to 12 carbon atoms inclusively in the cycloalkyl moiety. Such cycloalkylalkyl groups are exemplified by cyclopropylmethyl, cyclohexylethyl and the like.

“Amino” refers to the group —NH₂. Substituted amino refers to the group —NHR^{η}, NR^{η}R^{η}, and NR^{η}R^{η}R^{η}, where each R^{η} is independently selected from substituted or unsubstituted alkyl, cycloalkyl, cycloheteroalkyl, alkoxy, aryl, heteroaryl, heteroarylalkyl, acyl, alkoxycarbonyl, sulfanyl, sulfinyl, sulfonyl, and the like. Typical amino groups include, but are limited to, dimethylamino, diethylamino, trimethylammonium, triethylammonium, methylysulfonylamino, furanyl-oxy-sulfamino, and the like.

“Aminoalkyl” refers to an alkyl group in which one or more of the hydrogen atoms are replaced with one or more amino groups, including substituted amino groups.

“Aminocarbonyl” refers to —C(O)NH₂. Substituted aminocarbonyl refers to —C(O)NR^{η}R^{η}, where the amino group NR^{η}R^{η} is as defined herein.

“Oxy” refers to a divalent group —O—, which may have various substituents to form different oxy groups, including ethers and esters.

“Alkoxy” or “alkyloxy” are used interchangeably herein to refer to the group —ORC, wherein RC is an alkyl group, including optionally substituted alkyl groups.

“Carboxy” refers to —COOH.

“Carbonyl” refers to —C(O)—, which may have a variety of substituents to form different carbonyl groups including acids, acid halides, aldehydes, amides, esters, and ketones.

“Carboxyalkyl” refers to an alkyl in which one or more of the hydrogen atoms are replaced with one or more carboxy groups.

“Aminocarbonylalkyl” refers to an alkyl substituted with an aminocarbonyl group, as defined herein.

“Halogen” or “halo” refers to fluoro, chloro, bromo and iodo.

“Haloalkyl” refers to an alkyl group in which one or more of the hydrogen atoms are replaced with a halogen. Thus, the term “haloalkyl” is meant to include monohaloalkyls, dihaloalkyls, trihaloalkyls, etc. up to perhaloalkyls. For example, the expression “(C₁-C₂) haloalkyl” includes 1-fluoromethyl, difluoromethyl, trifluoromethyl, 1-fluoroethyl, 1,1-difluoroethyl, 1,2-difluoroethyl, 1,1,1 trifluoroethyl, perfluoroethyl, etc.

“Hydroxy” refers to —OH.

“Hydroxyalkyl” refers to an alkyl group in which in which one or more of the hydrogen atoms are replaced with one or more hydroxy groups.

“Thiol” or “sulfanyl” refers to —SH. Substituted thiol or sulfanyl refers to —S—R^{η}, where R^{η} is an alkyl, aryl or other suitable substituent.

“Alkylthio” refers to —SR^{ζ}, where R^{ζ} is an alkyl, which can be optionally substituted. Typical alkylthio group include, but are not limited to, methylthio, ethylthio, n-propylthio, and the like.

“Alkylthioalkyl” refers to an alkyl substituted with an alkylthio group, —SR^{ζ}, where R^{ζ} is an alkyl, which can be optionally substituted.

“Sulfonyl” refers to —SO₂—. Substituted sulfonyl refers to —SO₂—R^{η}, where R^{η} is an alkyl, aryl or other suitable substituent.

“Alkylsulfonyl” refers to —SO₂—R^{ζ}, where R^{ζ} is an alkyl, which can be optionally substituted. Typical alkylsulfonyl groups include, but are not limited to, methylsulfonyl, ethylsulfonyl, n-propylsulfonyl, and the like.

“Alkylsulfonylalkyl” refers to an alkyl substituted with an alkylsulfonyl group, —SO₂—R^{ζ}, where R^{ζ} is an alkyl, which can be optionally substituted.

“Heteroaryl” refers to an aromatic heterocyclic group of from 1 to 10 carbon atoms inclusively and 1 to 4 heteroatoms inclusively selected from oxygen, nitrogen and sulfur within the ring. Such heteroaryl groups can have a single ring (e.g., pyridyl or furyl) or multiple condensed rings (e.g., indolizinyl or benzothienyl).

“Heteroarylalkyl” refers to an alkyl substituted with a heteroaryl (i.e., heteroaryl-alkyl-groups), preferably having from 1 to 6 carbon atoms inclusively in the alkyl moiety and from 5 to 12 ring atoms inclusively in the heteroaryl moiety. Such heteroarylalkyl groups are exemplified by pyridylmethyl and the like.

“Heterocycle”, “heterocyclic” and interchangeably “heterocycloalkyl” refer to a saturated or unsaturated group having a single ring or multiple condensed rings, from 2 to 10 carbon ring atoms inclusively and from 1 to 4 hetero ring atoms inclusively selected from nitrogen, sulfur or oxygen within the ring. Such heterocyclic groups can have a single ring (e.g., piperidinyl or tetrahydrofuryl) or multiple condensed rings (e.g., indolinyl, dihydrobenzofuran or quinuclidinyl). Examples of heterocycles include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like.

“Heterocycloalkylalkyl” refers to an alkyl substituted with a heterocycloalkyl (i.e., heterocycloalkyl-alkyl-groups), preferably having from 1 to 6 carbon atoms inclusively in the alkyl moiety and from 3 to 12 ring atoms inclusively in the heterocycloalkyl moiety.

“Membered ring” is meant to embrace any cyclic structure. The number preceding the term “membered” denotes the number of skeletal atoms that constitute the ring. Thus, for example, cyclohexyl, pyridine, pyran and thiopyran are 6-membered rings and cyclopentyl, pyrrole, furan, and thiophene are 5-membered rings.

“Fused bicyclic ring” as used herein refers to both unsubstituted and substituted carbocyclic and/or heterocyclic ring moieties having 5 to 8 atoms in each ring, the rings having 2 common atoms.

“Optionally substituted” as used herein with respect to the foregoing chemical groups means that positions of the chemical group occupied by hydrogen can be substituted with another atom (unless otherwise specified) exemplified by, but not limited to carbon, oxygen, nitrogen, or sulfur, or a chemical group, exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; where preferred heteroatoms are oxygen, nitrogen, and sulfur. Additionally, where open valences exist on these substitute chemical groups they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further contemplated that the above substitutions can be made provided that replacing the hydrogen with the substituent does not introduce unacceptable instability to the molecules of the present invention, and is otherwise chemically reasonable. One of ordinary skill in the art would understand that with respect to any chemical group described as optionally substituted, only sterically practical and/or synthetically feasible chemical groups are meant to be included. “Optionally substituted” as used herein refers to all subsequent modifiers in a term or series of chemical groups. For example, in the term “optionally substituted arylalkyl,” the “alkyl” portion and the “aryl” portion of the molecule may or may not be substituted, and for the series “optionally substituted alkyl, cycloalkyl, aryl and heteroaryl,” the alkyl, cycloalkyl, aryl, and heteroaryl groups, independently of the others, may or may not be substituted.

“Reaction” as used herein refers to a process in which one or more substances or compounds or substrates is converted into one or more different substances, compounds, or processes.

### Conversion of Glycine and Trifluoroacetone to a Chiral Amino Alcohol

Compound (4), (S)-3-amino-1,1,1-trifluoro-2-methylpropan-2-ol, is an intermediate in the synthesis of a novel compound for the treatment of cystic fibrosis.

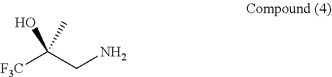

Enzymatic synthesis of compound (4), a β-hydroxy-α-amino alcohol, presents an attractive alternative to traditional chemical synthesis. Use of enzyme biocatalysts may reduce chemical waste and allow a one-pot synthesis method.

Generation of chiral β-amino alcohols via a β-hydroxy-α-amino acid intermediate using a two-enzyme cascade has been reported by others (Dückers et al. Appl Microbiol Biotechnol, 2010, 88:409-424). In the first step, threonine aldolase catalyzes the conversion of glycine (or another nucleophile) and acetaldehyde (or another electrophile) to threonine in a PLP-dependent mechanism. In the second step, an amino acid decarboxylase catalyzes the removal of the carboxyl group to generate the amino alcohol (which is also a PLP-dependent mechanism).

Many amino acid decarboxylases are known, with substrate specificity for various amino acids and amino acid analogs. Threonine aldolases accept a range of aldehyde electrophiles and amino acids as substrates, including alanine, serine, and cysteine. However, a threonine aldolase with activity on ketone substrates has not been reported.

Threonine aldolases catalyze the reaction between two achiral molecules, for example 1,1,1-trifluoroacetone and glycine, and produce a new molecule, a β-hydroxy-α-amino acid, with two chiral centers. The amino acid decarboxylase catalyzed reaction removes one of these chiral centers (the one at the α-position, assuming glycine is used as the nucleophile) during the subsequent decarboxylation step producing a chiral tertiary amino alcohol. There are two possible strategies for controlling the enantiomeric purity of the tertiary amino alcohol product. A threonine aldolase can be chosen or engineered to be highly selective at the β-position as the subsequent decarboxylation step does not affect the selectivity at this position. Alternatively, an amino acid decarboxylase can be chosen or engineered such that is has high selectivity at the β-position and will predominately decarboxylate only β-hydroxy-α-amino acids that have a particular enantiomeric configuration at the β-position. This catalyzed decarboxylation step can also occur in a dynamic kinetic resolution fashion, where the β-hydroxy-α-amino acid is concurrently interchanging between starting materials (trifluoroacetone and glycine) and product (the β-hydroxy-α-amino acid), as the threonine aldolase reaction is an equilibrium reaction. This could allow for a reaction providing up to 100% conversion to the tertiary amino alcohol product and with a product enantiomeric excess of up to 100%.

The present disclosure provides novel biocatalysts and associated methods of use for the synthesis of chiral β-amino alcohols via a β-hydroxy-α-amino acid intermediate using a two-enzyme cascade. The threonine aldolase biocatalysts of the present disclosure are engineered polypeptide variants of a homolog gene from Escherichia coli (SEQ ID NO:2) or a homolog gene from Sinorhizobium arboris (SEQ ID NO: 158). The amino acid decarboxylase biocatalysts of the present disclosure are engineered polypeptide variants of the wild-type gene from Planctomycetaceae bacterium (SEQ ID NO: 282). These engineered polypeptides are capable of catalyzing the conversion of trifluoroacetone (compound (1)) and glycine (compound (2)) to compound (4) via intermediate compound (3), as depicted below in Scheme 1.

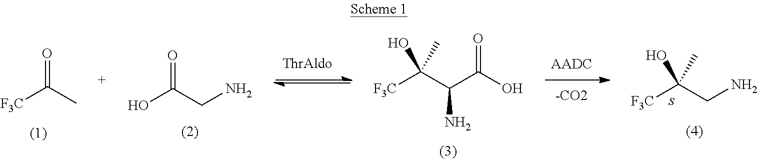

In some embodiments, the present disclosure provides engineered threonine aldolase and amino acid decarboxylase enzymes having improved activity in the production of compound (3) and/or compound (4) as compared to a reference polypeptide. In some embodiments, the engineered threonine aldolase enzymes and amino acid decarboxylase enzymes have the activity of Scheme 1.

As described herein, the engineered polypeptides exhibit stereoselectivity; thus, a threonine aldolase and amino acid decarboxylase of Scheme 1 can be used to establish one, or more, chiral centers of a product. The amino acid decarboxylases of the present disclosure produce (R) or (S) enantiomers according to the reaction in Scheme 2.

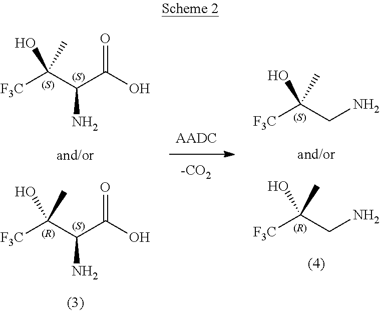

In some embodiments, the present disclosure provides engineered amino acid decarboxylase enzymes having improved stereoselectivity towards the (S)-amino alcohol product (compound (4)) as compared to a reference polypeptide. In some embodiments, the present disclosure provides an engineered polypeptide comprising an amino acid sequence having at least 80% sequence identity to an amino acid reference sequence of SEQ ID NO: 282 and further comprising one or more amino acid residue differences as compared to the reference amino acid sequence, wherein the engineered amino acid decarboxylase polypeptide has increased enantioselectivity towards compound (4).

### Engineered Threonine Aldolase and Amino Acid Decarboxylase Polypeptides

The present invention provides threonine aldolase and amino acid decarboxylase polypeptides, polynucleotides encoding the polypeptides, methods of preparing the polypeptides, and methods for using the polypeptides. Where the description relates to polypeptides, it is to be understood that it can describe the polynucleotides encoding the polypeptides.

Suitable reaction conditions under which the above-described improved properties of the engineered polypeptides carry out the desired reaction can be determined with respect to concentrations or amounts of polypeptide, substrate, co-substrate, buffer, solvent, pH, conditions including temperature and reaction time, and/or conditions with the polypeptide immobilized on a solid support, as further described below and in the Examples.

In some embodiments, exemplary engineered threonine aldolases with improved properties, particularly in the conversion of compound (1) and compound (2) to compound (3), comprise an amino acid sequence that has one or more residue differences as compared to SEQ ID NO: 2 at the residue positions indicated in Table 2-1.

In some embodiments, exemplary engineered threonine aldolase polypeptides with improved properties, particularly in the conversion of compound (1) and compound (2) to compound (3) and to compound (4), when paired with an amino acid decarboxylase, comprise an amino acid sequence that has one or more residue differences as compared to SEQ ID NO: 158 at the residue positions indicated in Table 4-1.

In some embodiments, exemplary engineered amino acid decarboxylase polypeptides with improved properties, particularly in the conversion of compound (3) to compound (4), when paired with a threonine aldolase in the presence of compound (1) and compound (2), comprise an amino acid sequence that has one or more residue differences as compared to SEQ ID NO: 282 at the residue positions indicated in Table 6-1.

The structure and function information for exemplary non-naturally occurring (or engineered) polypeptides of the present invention are based on the conversion of compound (1) and compound (2) to compound (3), the results of which are shown below in Tables 2-1 and 4-1, and the conversion of compound (1) and (2) to compound (3) and then to compound (4), the results of which are shown below in Table 6-1, as further described in the Examples. The odd numbered sequence identifiers (i.e., SEQ ID NOs) in these Tables refer to the nucleotide sequence encoding the amino acid sequence provided by the even numbered SEQ ID NOs in these Tables. Exemplary sequences are provided in the electronic sequence listing file accompanying this invention, which is hereby incorporated by reference herein. The amino acid residue differences are based on comparison to the reference sequence of SEQ ID NOs: 2, 158, and/or 282, as indicated.

Two threonine aldolases (SEQ ID NO:2 a homolog from Escherichia coli and SEQ ID NO: 158 a homolog from Sinorhizobium arboris) were selected based on their conversion of compound (1) and compound (2) to compound (3). SEQ ID NO: 1 is a codon-optimized polynucleotide for expression in Escherichia coli that was synthesized based on the polypeptide sequence of SEQ ID NO: 2, while SEQ ID NO: 157 is a codon-optimized polynucleotide for expression in Escherichia coli that was synthesized based on the polypeptide sequence of SEQ ID NO: 158. Additionally, an amino acid decarboxylase (SEQ ID NO: 282 from Planctomycetaceae bacterium) was selected based on conversion of compound (3) to compound (4) in a paired reaction with a threonine aldolase conversion of compound (1) and compound (2). SEQ ID NO: 281 is a codon-optimized polynucleotide for expression in Escherichia co/i that was synthesized based on the polypeptide sequence of SEQ ID NO: 281.

The activity of each engineered threonine aldolase and amino acid decarboxylase polypeptide relative to the reference polypeptide of SEQ ID NO: 2, 158, and/or 282 was determined as conversion of the substrates described in the Examples herein. In some embodiments, a shake flask powder (SFP) is used as a secondary screen to assess the properties of the engineered glucose dehydrogenases, the results of which are provided in the Examples. In some embodiments, the SFP forms provide a more purified powder preparation of the engineered polypeptides and can contain the engineered polypeptides that are up to about 30% of total protein.

In some embodiments, the specific enzyme properties are associated with the residues differences as compared to SEQ ID NO: 2, 158, and/or 282 at the residue positions indicated herein. In some embodiments, residue differences affecting polypeptide expression can be used to increase expression of the engineered threonine aldolase and/or amino acid decarboxylase.

In light of the guidance provided herein, it is further contemplated that any of the exemplary engineered polypeptides comprising the even-numbered sequences of SEQ ID NOs: 4-156, 160-280, and 284-366 find use as the starting amino acid sequence for synthesizing other engineered threonine aldolase and/or amino acid decarboxylase polypeptides, for example by subsequent rounds of evolution that incorporate new combinations of various amino acid differences from other polypeptides in Tables 2-1, 4-1, and 6-1, and other residue positions described herein. Further improvements may be generated by including amino acid differences at residue positions that had been maintained as unchanged throughout earlier rounds of evolution.

In some embodiments, the engineered threonine aldolase polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 2 and one or more residue differences as compared to SEQ ID NO: 2, selected from: 7/8/170/171, 7/170/171/172/195/196, 7/170/171/193, 8, 8/170/194/196, 10, 10/138/205/312, 10/167, 10/169/205, 10/198, 11/81/206, 11/206, 55/60, 55/60/171/174/253, 55/60/173, 55/60/247/250, 55/60/250, 55/60/250/253, 55/60/253, 55/170/253, 57/140/143/206, 60/247/253, 60/250/253, 60/253, 102, 138/167, 138/167/312, 138/169, 138/169/205, 138/169/276, 138/205, 138/245, 140/143/176/206, 140/143/206, 167/205, 169, 170, 170/171, 170/171/172/193/194, 170/172, 170/193/195/196, 170/196, 170/249, 170/253, 171, 171/172, 171/172/193/195/249, 171/174/201/253, 171/195, 171/201/253, 171/249, 171/253, 172, 173/247/250/324, 173/250/253, 174/211/253, 174/253, 191/253, 193, 193/194, 193/195, 194, 194/196, 195, 195/196, 196, 198/201/205, 201/205/246, 205, 206, 245, 249, 250/253, 253, and 276. In some embodiments, the engineered threonine aldolase polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 2 and one or more residue differences as compared to SEQ ID NO: 2, selected from: 7E/8L/170V/171A, 7E/170S/171A/193T, 7E/170V/171A/172G/195T/196H, 8L/170S/194T/196H, 8V, 10A, 10A/138C/205E/312P, 10A/167M, 10A/169H/205E, 10A/198T, 11L/81L/206A, 11L/206A, 55S/170L/253W, 55T/60S/253R, 55Y/60S, 55Y/60S/171G/174L/253W, 55Y/60S/173T, 55Y/60S/247L/250G, 55Y/60S/250G, 55Y/60S/250G/253W, 57S/140F/143S/206A, 60S/247L/253W, 60S/250G/253W, 60S/253W, 102H, 138A/167M, 138A/169H/205E, 138A/205E, 138C/167M, 138C/167M/312P, 138C/169H, 138C/169H/276H, 138C/245E, 140F/143S/206A, 140M/143S/176L/206A, 167M/205E, 169H, 170G/171A/172G/193T/194T, 170L/253W, 170S, 170S/172G, 170V/171A, 170V/193T/195T/196H, 170V/196H, 170V/249T, 171A, 171A/172G, 171A/172G/193T/195T/249M, 171A/174L/201C/253R, 171A/195T, 171A/249T, 171A/253W, 171G/201A/253R, 172G, 173T/247L/250G/324T, 173T/250G/253W, 174L/211C/253W, 174L/253W, 191A/253W, 193T, 193T/194T, 193T/195T, 194T, 194T/196H, 195T, 195T/196H, 196H, 198T/201P/205E, 201P/205E/246F, 205E, 206A, 245E, 249T, 250G/253W, 253R, 253W, and 276H. In some embodiments, the engineered threonine aldolase polypeptide comprises an amino acid sequence having at least 80%, 85%, 86%, 87%, 88%, 89%, 90%, 91%, 92%, 93%, 94%, 95% 96% 97%, 98%, 99% or more sequence identity to reference sequence SEQ ID NO: 2 and one or more residue differences as compared to SEQ ID NO: 2, selected from: D7E/T8L/1170V/F171A, D7E/I170S/F171A/I193T, D7E/1170V/F171A/N172G/L195T/S196H, T8L/I170S/C194T/S196H, T8V, T10A, T10A/G138C/S205E/H312P, T10A/G167M, T10A/R169H/S205E, T10A/G198T, R11L/A81L/L206A, R11L/L206A, L55S/1170L/H253W, L55T/Q60S/H253R, L55Y/Q60S, L55Y/Q60S/F171G/V174L/H253W, L55Y/Q60S/A173T, L55Y/Q60S/A247L/Q250G, L55Y/Q60S/Q250G, L55Y/Q60S/Q250G/H253W, T57S/N140F/V143S/L206A, Q60S/A247L/H253W, Q60S/Q250G/H253W, Q60S/H253W, P102H, G138A/G167M, G138A/R169H/S205E, G138A/S205E, G138C/G167M, G138C/G167M/H312P, G138C/R169H, G138C/R169H/N276H, G138C/N245E, N140F/V143S/L206A, N140M/V143S/A176L/L206A, G167M/S205E, R169H, I170G/F171A/N172G/1193T/C194T, 1170L/H253W, 1170S, 1170S/N172G, 1170V/F171A, I170V/1193T/L195T/S196H, I170V/S196H, I170V/L249T, F171A, F171A/N172G, F171A/N172G/1193T/L195T/L249M, F171A/V174L/T201C/H253R, F171A/L195T, F171A/L249T, F171A/H253W, F171G/T201A/H253R, N172G, A173T/A247L/Q250G/A324T, A173T/Q250G/H253W, V174L/R211C/H253W, V174L/H253W, F191A/H253W, 1193T, I193T/C194T, 1193T/L195T, C194T, C194T/S196H, L195T, L195T/S196H, S196H, G198T/T201P/S205E, T201P/S205E/V246F, S205E, L206A, N245E, L249T, Q250G/H253W, H253R, H253W, and N276H.

In some embodiments, the engineered threonine aldolase polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 158 and one or more residue differences as compared to SEQ ID NO: 158, selected from: 15/268/317, 128, 141/207/210/254/257/258, 141/207/210/254/258, 141/207/255/258, 141/211/212/254, 141/211/257/258, 141/212/254/257/258, 141/212/257/258, 141/254/255/257/258, 141/254/255/258, 141/254/257/258, 141/254/258, 141/255/257/258, 141/257/258, 141/258, 172/174/175, 172/174/175/200/203/205/245, 172/174/175/245, 172/174/175/245/248, 172/174/199/200/203/205/248, 172/174/245, 172/174/248, 172/175, 172/199/203/204/206, 174/175, 174/175/199/200/203/206/245/248, 174/175/199/200/205, 174/175/199/203/204/205, 174/175/245, 174/175/245/248, 174/245/248, 175/176/199, 175/245, 178/181/211/254/255/258, 178/207/211/254/255/257/258, 199/200/203/204/206/245, 199/200/203/206/248, 199/200/204, 199/200/206, 199/203/204, 199/203/204/206/245/248, 203/245/248, 207/210/211/255/257, 207/210/212/257, 207/210/255, 207/211, 207/211/212/254/255/257/258, 207/211/257/258, 207/212/255/258, 210/212/258, 210/254, 211/254/257/258, 211/254/258, 211/257/258, 245/248, 254/255/257/258, 254/255/258, 254/257, and 258. In some embodiments, the engineered threonine aldolase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 158 and one or more residue differences as compared to SEQ ID NO: 158, selected from: 15L/268Q/317W, 128V, 141S/207L/210R/254M/258Q, 141S/207L/255K/258S, 141S/207M/210R/254M/257Y/258Q, 141S/211G/212G/254M, 141S/211S/257Y/258Q, 141S/212G/254M/257Y/258S, 141S/212G/257Y/258Q, 141S/254M/255K/257Y/258Q, 141S/254M/255K/258Q, 141S/254M/257Y/258Q, 141S/254M/258Q, 141S/255K/257Y/258Q, 141S/257Y/258Q, 141S/258S, 172M/174H/175I/245K/248A, 172M/174H/175V, 172M/174H/175V/200K/203S/205E/245K, 172M/174H/175V/245K, 172M/174H/199T/200K/203S/205E/248A, 172M/174H/245K, 172M/174H/248A, 172M/175V, 172M/199T/203S/204E/206L, 174H/175I/199T/200K/203S/206L/245K/248A, 174H/175I/199T/200K/205E, 174H/175I/245K, 174H/175I/245K/248A, 174H/175V, 174H/175V/199T/203S/204E/205E, 174H/245K/248A, 175I/245K, 175S/176Q/199T, 178G/181C/211S/254M/255K/258Q, 178G/207L/211S/254M/255K/257Y/258S, 199T/200K/203S/204E/206L/245K, 199T/200K/203S/206L/248A, 199T/200K/204E, 199T/200K/206L, 199T/203S/204E, 199T/203S/204E/206L/245K/248A, 203S/245K/248A, 207L/212G/255K/258Q, 207M/210P/211Q/255K/257Y, 207M/210R/212G/257Y, 207M/210R/255K, 207M/211G, 207M/211G/212G/254M/255K/257Y/258Q, 207M/211S, 207M/211S/257Y/258Q, 210R/212G/258Q, 210R/254M, 211A/254M/258S, 211S/254M/257Y/258Q, 211S/257Y/258Q, 245K/248A, 254M/255K/257Y/258Q, 254M/255K/258Q, 254M/257Y, and 258Q. In some embodiments, the engineered threonine aldolase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 158 and one or more residue differences as compared to SEQ ID NO: 158, selected from: 115L/L268Q/L317W, N128V, Q141S/C207L/A210R/W254M/A258Q, Q141S/C207L/L255K/A258S, Q141S/C207M/A210R/W254M/L257Y/A258Q, Q141S/E211G/A212G/W254M, Q141S/E211S/L257Y/A258Q, Q141S/A212G/W254M/L257Y/A258S, Q141S/A212G/L257Y/A258Q, Q141S/W254M/L255K/L257Y/A258Q, Q141S/W254M/L255K/A258Q, Q141S/W254M/L257Y/A258Q, Q141S/W254M/A258Q, Q141S/L255K/L257Y/A258Q, Q141S/L257Y/A258Q, Q141S/A258S, G172M/R174H/F175I/F245K/Y248A, G172M/R174H/F175V, G172M/R174H1F175V/F200K/T203S/N205E/F245K, G172M/R174H/F175V/F245K, G172M/R174H1S199T/F200K/T203S/N205E/Y248A, G172M/R174H/F245K, G172M/R174H/Y248A, G172M/F175V, G172M/S199T/T203S/K204E/G206L, R174H/F175I/S199T/F200K/T203S/G206L/F245K/Y248A, R174H/F175I/S199T/F200K/N205E, R174H/F175I/F245K, R174H/F175I/F245K/Y248A, R174H/F175V, R174H/F175V/S199T/T203S/K204E/N205E, R174H/F245K/Y248A, F175I/F245K, F175S/A176Q/S199T, A178G/S181C/E211S/W254M/L255K/A258Q, A178G/C207L/E211S/W254M/L255K/L257Y/A258S, S199T/F200K/T203S/K204E/G206L/F245K, S199T/F200K/T203S/G206L/Y248A, S199T/F200K/K204E, S199T/F200K/G206L, S199T/T203S/K204E, S199T/T203S/K204E/G206L/F245K/Y248A, T203S/F245K/Y248A, C207L/A212G/L255K/A258Q, C207M/A210P/E211Q/L255K/L257Y, C207M/A210R/A212G/L257Y, C207M/A210R/L255K, C207M/E211G, C207M/E211G/A212G/W254M/L255K/L257Y/A258Q, C207M/E211S, C207M/E211S/L257Y/A258Q, A210R/A212G/A258Q, A210R/W254M, E211A/W254M/A258S, E211S/W254M/L257Y/A258Q, E211S/L257Y/A258Q, F245K/Y248A, W254M/L255K/L257Y/A258Q, W254M/L255K/A258Q, W254M/L257Y, and A258Q.

In some embodiments, the engineered amino acid decarboxylase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 282 and one or more residue differences as compared to SEQ ID NO: 282, selected from: 48, 49, 55/74/110/121/194/233/253, 55/74/110/194/233/281/324, 55/74/110/211, 55/74/121/233/253/324, 55/74/194/233, 55/194/211/233/253, 55/211/253/324, 56, 63/398, 66/86/198/235/329, 66/198/202/290/316/329, 66/202/290/329, 66/290/329, 72, 74/110/121/194, 74/110/233/324, 74/110/324, 74/121/194/233/253, 74/121/194/233/324, 74/194, 74/194/233, 74/194/253, 80, 86, 86/162/186/187/202/203/235/329, 103, 135, 162, 162/187/202/235/252/290/316, 174, 183, 198, 245, 248, 258, 279, 280, and 350. In some embodiments, the engineered amino acid decarboxylase comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 282 and one or more residue differences as compared to SEQ ID NO: 282, selected from: 48V, 49N, 55I/74F/110Y/121L/194L/233I/253K, 55I/74F/110Y/194L/233I/281H/324W, 55I/74F/110Y/211S, 55I/74F/121L/233I/253K/324W, 55I/74F/194L/233I, 55I/194L/211S/233I/253K, 55I/211S/253K/324W, 56D, 63T/398L, 66N/86F/198G/235V/329S, 66N/198G/202Y/290I/316L/329S, 66N/202Y/290I/329S, 66N/290I/329S, 72A, 72E, 74F/110Y/121L/194L, 74F/110Y/233I/324W, 74F/110Y/324W, 74F/121L/194L/233I/253K, 74F/121L/194L/233I/324W, 74F/194L, 74F/194L/233I, 74F/194L/253K, 80K, 86F/162I/186D/187I/202Y/203Y/235V/329S, 86T, 103G, 135I, 162I/187I/202Y/235V/252A/290I/316L, 162L, 174C, 183V, 198S, 245E, 245W, 248S, 258S, 279P, 280A, and 350E. In some embodiments, the engineered amino acid decarboxylase polypeptide comprises an amino acid sequence having at least 80%, 85%, 86%, 87%, 88%, 89%, 90%, 91%, 92%, 93%, 94%, 95%, 96%, 97%, 98%, 99% or more sequence identity to reference sequence SEQ ID NO: 282 and one or more residue differences as compared to SEQ ID NO: 282, selected from: L48V, P49N, V55I/I74F/F110Y/A121L/F194L/L233I/R253K, V55I/174F/F110Y/F194L/L233I/1281H/R324W, V55I/174F/F110Y/A211S, V55I/174F/A121L/L233I/R253K/R324W, V55I/I74F/F194L/L233I, V55I/F194L/A211S/L233I/R253K, V55I/A211S/R253K/R324W, N56D, A63T/P398L, S66N/M86F/A198G/1235V/A329S, S66N/A198G/S202Y/V290I/F316L/A329S, S66N/S202Y/V290I/A329S, S66N/V290I/A329S, R72A, R72E, 174F/F110Y/A121L/F194L, 174F/F110Y/L233I/R324W, I74F/F110Y/R324W, I74F/A121L/F194L/L233I/R253K, 174F/A121L/F194L/L233I/R324W, I74F/F194L, 174F/F194L/L233I, I74F/F194L/R253K, A80K, M86F/M162I/N186D/V187I/S202Y/H203Y/I235V/A329S, M86T, E103G, L135I, M162I/V187I/S202Y/1235V/T252A/V290I/F316L, M162L, A174C, G183V, A198S, I245E, I245W, G248S, E258S, N279P, G280A, and N350E.

As will be appreciated by the skilled artisan, in some embodiments, one or a combination of residue differences above that is selected can be kept constant (i.e., maintained) in the engineered threonine aldolase and/or amino acid decarboxylase as a core feature, and additional residue differences at other residue positions incorporated into the sequence to generate additional engineered threonine aldolase and/or amino acid decarboxylase polypeptides with improved properties. Accordingly, it is to be understood for any engineered threonine aldolase and/or amino acid decarboxylase containing one or a subset of the residue differences above, the present invention contemplates other engineered threonine aldolases and/or amino acid decarboxylases that comprise the one or subset of the residue differences, and additionally one or more residue differences at the other residue positions disclosed herein.

As noted above, the engineered threonine aldolase polypeptides are also capable of converting substrates (e.g., compound (1) and compound (2)) to products (e.g., compound (3)). In some embodiments, the engineered threonine aldolase polypeptide is capable of converting the substrate compounds to the product compound with at least 1.2 fold, 1.5 fold, 2 fold, 3 fold, 4 fold, 5 fold, 10 fold, 20 fold, 30 fold, 40 fold, 50 fold, 60 fold, 70 fold, 80 fold, 90 fold, 100 fold, or more activity relative to the activity of the reference polypeptide of SEQ ID NO: 2 and/or 158.

Similarly, the engineered amino acid decarboxylase polypeptides are also capable of converting substrates (e.g., compound (3)) to products (e.g., compound (4)). In some embodiments, the engineered amino acid decarboxylase polypeptide is capable of converting the substrate compounds to the product compound with at least 1.2 fold, 1.5 fold, 2 fold, 3 fold, 4 fold, 5 fold, 10 fold, 20 fold, 30 fold, 40 fold, 50 fold, 60 fold, 70 fold, 80 fold, 90 fold, 100 fold, or more activity relative to the activity of the reference polypeptide of SEQ ID NO: 282.

In some embodiments, the engineered threonine aldolase polypeptide capable of converting the substrate compounds to the product compounds with at least 2 fold the activity relative to SEQ ID NO: 2 or 158, comprises an amino acid sequence selected from: the even-numbered sequences in SEQ ID NOs: 4-156 and 160-280.

In some embodiments, the engineered amino acid decarboxylase polypeptide capable of converting the substrate compounds to the product compounds with at least 2 fold the activity relative to SEQ ID NO: 282, comprises an amino acid sequence selected from: the even-numbered sequences in SEQ ID NOs: 284-366.

In some embodiments, the engineered threonine aldolase has an amino acid sequence comprising one or more residue differences as compared to SEQ ID NO: 2 or 158, increases expression of the engineered threonine aldolase activity in a bacterial host cell, particularly in E. coli.

In some embodiments, the engineered amino acid decarboxylase has an amino acid sequence comprising one or more residue differences as compared to SEQ ID NO: 282, increases expression of the engineered amino acid decarboxylase activity in a bacterial host cell, particularly in E. coli.

In some embodiments, the engineered threonine aldolase or amino acid decarboxylase polypeptide with improved properties has an amino acid sequence comprising a sequence selected from the even-numbered sequences in the range of SEQ ID NOs: 4-156, 160-280, and 284-366.

In some embodiments, the engineered threonine aldolase, comprises an amino acid sequence having at least 80%, 85%, 86%, 87%, 88%, 89%, 90%, 91%, 92%, 93%, 94% 95%, 96%, 97%, 98%, or 99% identity to one of the even-numbered sequences in the range of SEQ ID NOs: 4-156 or 160-280, and the amino acid residue differences as compared to SEQ ID NO: 2 or 158, present in any one of the even-numbered sequences in the range of SEQ ID NOs: 4-156 or 160-280, as provided in the Examples.

In some embodiments, the engineered amino acid decarboxylase, comprises an amino acid sequence having at least 80%, 85%, 86%, 87%, 88%, 89%, 90%, 91%, 92%, 93%, 94%, 95%, 96%, 97%, 98%, or 99% identity to one of the even-numbered sequences in the range of SEQ ID NOs: 284-366, and the amino acid residue differences as compared to SEQ ID NO: 282, present in any one of the even-numbered sequences in the range of SEQ ID NOs: 284-366, as provided in the Examples.

In addition to the residue positions specified above, any of the engineered threonine aldolase and/or amino acid decarboxylase polypeptides disclosed herein can further comprise other residue differences relative to SEQ ID NO: 2, 158, or 282, at other residue positions (i.e., residue positions other than those included herein). Residue differences at these other residue positions can provide for additional variations in the amino acid sequence without adversely affecting the ability of the polypeptide to carry out the conversion of substrate to product. Accordingly, in some embodiments, in addition to the amino acid residue differences present in any one of the engineered threonine aldolase and/or amino acid decarboxylase polypeptides selected from the even-numbered sequences in the range of SEQ ID NOs: 4-156, 160-280, and 284-366, the sequence can further comprise 1-2, 1-3, 1-4, 1-5, 1-6, 1-7, 1-8, 1-9, 1-10, 1-11, 1-12, 1-14, 1-15, 1-16, 1-18, 1-20, 1-22, 1-24, 1-26, 1-30, 1-35, 1-40, 1-45, or 1-50 residue differences at other amino acid residue positions as compared to the SEQ ID NO: 2, 158, or 282. In some embodiments, the number of amino acid residue differences as compared to the reference sequence can be 1, 2, 3, 4, 5, 6, 7, 8, 9, 10, 11, 12, 13, 14, 15, 16, 17, 18, 19, 20, 21, 22, 23, 24, 25, 30, 30, 35, 40, 45 or 50 residue positions. In some embodiments, the number of amino acid residue differences as compared to the reference sequence can be 1, 2, 3, 4, 5, 6, 7, 8, 9, 10, 11, 12, 13, 14, 15, 16, 18, 20, 21, 22, 23, 24, or 25 residue positions. The residue differences at these other positions can be conservative changes or non-conservative changes. In some embodiments, the residue differences can comprise conservative substitutions and non-conservative substitutions as compared to the threonine aldolase and/or amino acid decarboxylase polypeptide of SEQ ID NOs: 2, 158, or 282.

In some embodiments, the present invention also provides engineered polypeptides that comprise a fragment of any of the engineered threonine aldolase or amino acid decarboxylase polypeptides described herein that retains the functional activity and/or improved property of that engineered threonine aldolase or amino acid decarboxylase. Accordingly, in some embodiments, the present invention provides a polypeptide fragment capable of converting substrate to product under suitable reaction conditions, wherein the fragment comprises at least about 90%, 95%, 96%, 97%, 98%, or 99% of a full-length amino acid sequence of an engineered threonine aldolase or amino acid decarboxylase of the present invention, such as an exemplary engineered threonine aldolase or amino acid decarboxylase polypeptide selected from the even-numbered sequences in the range of SEQ ID NOs: 4-156, 160-280, and 284-366. In some embodiments, the engineered threonine aldolase or amino acid decarboxylase can have an amino acid sequence comprising a deletion in any one of the threonine aldolase or amino acid decarboxylase polypeptide sequences described herein, such as the exemplary engineered polypeptides of the even-numbered sequences in the range of SEQ ID NOs: 4-156, 160-280, and 284-366.

Thus, for each and every embodiment of the engineered threonine aldolase or amino acid decarboxylase polypeptides of the invention, the amino acid sequence can comprise deletions of one or more amino acids, 2 or more amino acids, 3 or more amino acids, 4 or more amino acids, 5 or more amino acids, 6 or more amino acids, 8 or more amino acids, 10 or more amino acids, 15 or more amino acids, or 20 or more amino acids, up to 10% of the total number of amino acids, up to 20% of the total number of amino acids, or up to 30% of the total number of amino acids of the threonine aldolase or amino acid decarboxylase polypeptides, where the associated functional activity and/or improved properties of the engineered threonine aldolase or amino acid decarboxylase described herein are maintained. In some embodiments, the deletions can comprise 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residues. In some embodiments, the number of deletions can be 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residues. In some embodiments, the deletions can comprise deletions of 1, 2, 3, 4, 5, 6, 7, 8, 9, 10, 11, 12, 13, 14, 15, 16, 18, 20, 21, 22, 23, 24, or 25 amino acid residues.

In some embodiments, the engineered threonine aldolase or amino acid decarboxylase polypeptide described herein can have an amino acid sequence comprising an insertion as compared to any one of the engineered threonine aldolase or amino acid decarboxylase polypeptides described herein, such as the exemplary engineered polypeptides of the even-numbered sequences in the range of SEQ ID NOs: 4-156, 160-280, and 284-366. Thus, for each and every embodiment of the threonine aldolase or amino acid decarboxylase polypeptides of the disclosure, the insertions can comprise one or more amino acids, 2 or more amino acids, 3 or more amino acids, 4 or more amino acids, 5 or more amino acids, 6 or more amino acids, 8 or more amino acids, 10 or more amino acids, 15 or more amino acids, 20 or more amino acids, 30 or more amino acids, 40 or more amino acids, or 50 or more amino acids, where the associated functional activity and/or improved properties of the engineered threonine aldolase or amino acid decarboxylase described herein is maintained. The insertions can be to amino or carboxy terminus, or internal portions of the threonine aldolase or amino acid decarboxylase polypeptide.

In some embodiments, the engineered threonine aldolase or amino acid decarboxylase herein can have an amino acid sequence comprising a sequence selected from the even-numbered sequences in the range of SEQ ID NOs: 4-156, 160-280, and 284-366, and optionally one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the number of amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions can be conservative or non-conservative substitutions.

In the above embodiments, the suitable reaction conditions for the engineered polypeptides are provided in Tables 2-2, 4-2, and 6-2, and as described in the Examples herein.

In some embodiments, the polypeptides of the present invention are fusion polypeptides in which the engineered polypeptides are fused to other polypeptides, such as, by way of example and not limitation, antibody tags (e.g., myc epitope), purification sequences (e.g., His tags for binding to metals), and cell localization signals (e.g., secretion signals). Thus, the engineered polypeptides described herein can be used with or without fusions to other polypeptides.

It is to be understood that the polypeptides described herein are not restricted to the genetically encoded amino acids. In addition to the genetically encoded amino acids, the polypeptides described herein may be comprised, either in whole or in part, of naturally occurring and/or synthetic non-encoded amino acids. Certain commonly encountered non-encoded amino acids of which the polypeptides described herein may be comprised include, but are not limited to: the D-stereomers of the genetically-encoded amino acids; 2,3-diaminopropionic acid (Dpr); α-aminoisobutyric acid (Aib); ε-aminohexanoic acid (Aha); δ-aminovaleric acid (Ava); N-methylglycine or sarcosine (MeGly or Sar); ornithine (Orn); citrulline (Cit); t-butylalanine (Bua); t-butylglycine (Bug); N-methylisoleucine (MeIle); phenylglycine (Phg); cyclohexylalanine (Cha); norleucine (Nle); naphthylalanine (Nal); 2-chlorophenylalanine (Ocf); 3-chlorophenylalanine (Mcf); 4-chlorophenylalanine (Pcf); 2-fluorophenylalanine (Off); 3-fluorophenylalanine (Mff); 4-fluorophenylalanine (Pff); 2-bromophenylalanine (Obf); 3-bromophenylalanine (Mbf); 4-bromophenylalanine (Pbf); 2-methylphenylalanine (Omf); 3-methylphenylalanine (Mmf); 4-methylphenylalanine (Pmf); 2-nitrophenylalanine (Onf); 3-nitrophenylalanine (Mnf); 4-nitrophenylalanine (Pnf); 2-cyanophenylalanine (Ocf); 3-cyanophenylalanine (Mcf); 4-cyanophenylalanine (Pcf); 2-trifluoromethylphenylalanine (Otf); 3-trifluoromethylphenylalanine (Mtf); 4-trifluoromethylphenylalanine (Ptf); 4-aminophenylalanine (Paf); 4-iodophenylalanine (Pif); 4-aminomethylphenylalanine (Pamf); 2,4-dichlorophenylalanine (Opef); 3,4-dichlorophenylalanine (Mpcf); 2,4-difluorophenylalanine (Opff); 3,4-difluorophenylalanine (Mpff); pyrid-2-ylalanine (2pAla); pyrid-3-ylalanine (3pAla); pyrid-4-ylalanine (4pAla); naphth-1-ylalanine (1nAla); naphth-2-ylalanine (2nAla); thiazolylalanine (taAla); benzothienylalanine (bAla); thienylalanine (tAla); furylalanine (fAla); homophenylalanine (hPhe); homotyrosine (hTyr); homotryptophan (hTrp); pentafluorophenylalanine (5ff); styrylkalanine (sAla); authrylalanine (aAla); 3,3-diphenylalanine (Dfa); 3-amino-5-phenypentanoic acid (Afp); penicillamine (Pen); 1,2,3,4-tetrahydroisoquinoline-3-carboxylic acid (Tic); β-2-thienylalanine (Thi); methionine sulfoxide (Mso); N(w)-nitroarginine (nArg); homolysine (hLys); phosphonomethylphenylalanine (pmPhe); phosphoserine (pSer); phosphothreonine (pThr); homoaspartic acid (hAsp); homoglutanic acid (hGlu); 1-aminocyclopent-(2 or 3)-ene-4 carboxylic acid; pipecolic acid (PA), azetidine-3-carboxylic acid (ACA); 1-aminocyclopentane-3-carboxylic acid; allylglycine (aGly); propargylglycine (pgGly); homoalanine (hAla); norvaline (nVal); homoleucine (hLeu), homovaline (hVal); homoisoleucine (hIle); homoarginine (hArg); N-acetyl lysine (AcLys); 2,4-diaminobutyric acid (Dbu); 2,3-diaminobutyric acid (Dab); N-methylvaline (MeVal); homocysteine (hCys); homoserine (hSer); hydroxyproline (Hyp) and homoproline (hPro). Additional non-encoded amino acids of which the polypeptides described herein may be comprised will be apparent to those of skill in the art (See e.g., the various amino acids provided in Fasman, CRC Practical Handbook of Biochemistry and Molecular Biology, CRC Press, Boca Raton, FL, pp. 3-70 [1989], and the references cited therein, all of which are incorporated by reference). These amino acids may be in either the L- or D-configuration.

Those of skill in the art will recognize that amino acids or residues bearing side chain protecting groups may also comprise the polypeptides described herein. Non-limiting examples of such protected amino acids, which in this case belong to the aromatic category, include (protecting groups listed in parentheses), but are not limited to: Arg(tos), Cys(methylbenzyl), Cys (nitropyridinesulfenyl), Glu(δ-benzylester), Gln(xanthyl), Asn(N-δ-xanthyl), His(bom), His(benzyl), His(tos), Lys(fmoc), Lys(tos), Ser(O-benzyl), Thr (O-benzyl) and Tyr(O-benzyl).

Non-encoding amino acids that are conformationally constrained of which the polypeptides described herein may be composed include, but are not limited to, N-methyl amino acids (L-configuration); 1-aminocyclopent-(2 or 3)-ene-4-carboxylic acid; pipecolic acid; azetidine-3-carboxylic acid; homoproline (hPro); and 1-aminocyclopentane-3-carboxylic acid.

In some embodiments, the engineered polypeptides can be in various forms, for example, such as an isolated preparation, as a substantially purified enzyme, whole cells transformed with gene(s) encoding the enzyme, and/or as cell extracts and/or lysates of such cells. The enzymes can be lyophilized, spray-dried, precipitated or be in the form of a crude paste, as further discussed below.

In some embodiments, the engineered polypeptides can be in the form of a biocatalytic composition. In some embodiments, the biocatalytic composition comprises (a) a means for conversion of a ketone compound and an amino acid compound to a chiral R-amino alcohols via a β-hydroxy-α-amino acid intermediate by contact with a threonine aldolase polypeptide and an amino acid decarboxylase polypeptide and (b) a suitable cofactor. In some embodiments, the biocatalytic composition comprises a threonine aldolase having activity on a ketone substrate. In some embodiments, the biocatalytic composition comprises an amino acid decarboxylase having activity on a β-hydroxy-α-amino acid. In some further embodiments, the biocatalytic composition comprises a threonine aldolase and an amino acid decarboxylase that catalyze a multistep reaction pathway in a single pot. In some embodiments, the biocatalytic composition comprises a PLP (pyridoxal phosphate) cofactor.

In some embodiments, the engineered polypeptides can be provided on a solid support, such as a membrane, resin, solid carrier, or other solid phase material. A solid support can be composed of organic polymers such as polystyrene, polyethylene, polypropylene, polyfluoroethylene, polyethyleneoxy, and polyacrylamide, as well as co-polymers and grafts thereof. A solid support can also be inorganic, such as glass, silica, controlled pore glass (CPG), reverse phase silica or metal, such as gold or platinum. The configuration of a solid support can be in the form of beads, spheres, particles, granules, a gel, a membrane or a surface. Surfaces can be planar, substantially planar, or non-planar. Solid supports can be porous or non-porous, and can have swelling or non-swelling characteristics. A solid support can be configured in the form of a well, depression, or other container, vessel, feature, or location.

In some embodiments, the engineered threonine aldolase and/or amino acid decarboxylase polypeptides of the present invention can be immobilized on a solid support such that they retain their improved activity, and/or other improved properties relative to the reference polypeptide of SEQ ID NO: 2, 158, or 282. In such embodiments, the immobilized polypeptides can facilitate the biocatalytic conversion of the substrate compounds or other suitable substrates to the product and after the reaction is complete are easily retained (e.g., by retaining beads on which polypeptide is immobilized) and then reused or recycled in subsequent reactions. Such immobilized enzyme processes allow for further efficiency and cost reduction. Accordingly, it is further contemplated that any of the methods of using the threonine aldolase and/or amino acid decarboxylase polypeptides of the present invention can be carried out using the threonine aldolase and/or amino acid decarboxylase polypeptides bound or immobilized on a solid support.

Methods of enzyme immobilization are well-known in the art. The engineered polypeptides can be bound non-covalently or covalently. Various methods for conjugation and immobilization of enzymes to solid supports (e.g., resins, membranes, beads, glass, etc.) are well known in the art (See e.g., Yi et al., Proc. Biochem., 42(5): 895-898 [2007]; Martin et al., Appl. Microbiol. Biotechnol., 76(4): 843-851 [2007]; Koszelewski et al., J. Mol. Cat. B: Enzymatic, 63: 39-44 [2010]; Truppo et al., Org. Proc. Res. Dev., published online: dx.doi.org/10.1021/op200157c; Hermanson, Bioconjugate Techniques, 2^{nd} ed., Academic Press, Cambridge, MA [2008]; Mateo et al., Biotechnol. Prog., 18(3):629-34 [2002]; and “Bioconjugation Protocols: Strategies and Methods,” In Methods in Molecular Biology, Niemeyer (ed.), Humana Press, New York, NY [2004]; the disclosures of each which are incorporated by reference herein). Solid supports useful for immobilizing the engineered threonine aldolase and/or amino acid decarboxylase of the present invention include but are not limited to beads or resins comprising polymethacrylate with epoxide functional groups, polymethacrylate with amino epoxide functional groups, styrene/DVB copolymer or polymethacrylate with octadecyl functional groups. Exemplary solid supports useful for immobilizing the engineered threonine aldolase and/or amino acid decarboxylase polypeptides of the present invention include, but are not limited to, chitosan beads, Eupergit C, and SEPABEADs (Mitsubishi), including the following different types of SEPABEAD: EC-EP, EC-HFA/S, EXA252, EXE119 and EXE120.

In some embodiments, the polypeptides described herein are provided in the form of kits. The enzymes in the kits may be present individually or as a plurality of enzymes. The kits can further include reagents for carrying out the enzymatic reactions, substrates for assessing the activity of enzymes, as well as reagents for detecting the products. The kits can also include reagent dispensers and instructions for use of the kits.

In some embodiments, the kits of the present invention include arrays comprising a plurality of different threonine aldolase or amino acid decarboxylase polypeptides at different addressable position, wherein the different polypeptides are different variants of a reference sequence each having at least one different improved enzyme property. In some embodiments, a plurality of polypeptides immobilized on solid supports are configured on an array at various locations, addressable for robotic delivery of reagents, or by detection methods and/or instruments. The array can be used to test a variety of substrate compounds for conversion by the polypeptides. Such arrays comprising a plurality of engineered polypeptides and methods of their use are known in the art (See e.g., WO2009/008908A2).

Polynucleotides Encoding Engineered Threonine Aldolase and/or Amino Acid Decarboxylase, Expression Vectors and Host Cells

In another aspect, the present invention provides polynucleotides encoding the engineered threonine aldolase or amino acid decarboxylase polypeptides described herein. The polynucleotides may be operatively linked to one or more heterologous regulatory sequences that control gene expression to create a recombinant polynucleotide capable of expressing the polypeptide. Expression constructs containing a heterologous polynucleotide encoding the engineered threonine aldolase or amino acid decarboxylase are introduced into appropriate host cells to express the corresponding threonine aldolase or amino acid decarboxylase polypeptide.

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode the improved threonine aldolase and/or amino acid decarboxylase enzymes. Thus, having knowledge of a particular amino acid sequence, those skilled in the art could make any number of different nucleic acids by simply modifying the sequence of one or more codons in a way which does not change the amino acid sequence of the protein. In this regard, the present invention specifically contemplates each and every possible variation of polynucleotides that could be made encoding the polypeptides described herein by selecting combinations based on the possible codon choices, and all such variations are to be considered specifically disclosed for any polypeptide described herein, including the amino acid sequences presented in Tables 2-1, 4-1, and 6-1, and disclosed in the sequence listing incorporated by reference herein as the even-numbered sequences in the range of SEQ ID NOs: 4-156, 158-280, and 284-366.

In various embodiments, the codons are preferably selected to fit the host cell in which the protein is being produced. For example, preferred codons used in bacteria are used to express the gene in bacteria; preferred codons used in yeast are used for expression in yeast; and preferred codons used in mammals are used for expression in mammalian cells. In some embodiments, all codons need not be replaced to optimize the codon usage of the threonine aldolase or amino acid decarboxylase since the natural sequence will comprise preferred codons and because use of preferred codons may not be required for all amino acid residues. Consequently, codon optimized polynucleotides encoding the threonine aldolase or amino acid decarboxylase enzymes may contain preferred codons at about 40%, 50%, 60%, 70%, 80%, or greater than 90% of codon positions of the full length coding region.

In some embodiments, the polynucleotide comprises a codon optimized nucleotide sequence encoding the threonine aldolase or amino acid decarboxylase polypeptide amino acid sequence, as represented by SEQ ID NO: 2, 158, and/or 282. In some embodiments, the polynucleotide has a nucleic acid sequence comprising at least 80%, 85%, 90%, 91%, 92%, 93%, 94%, 95%, 96%, 97%, 98%, 99% or more identity to the codon optimized nucleic acid sequences encoding the even-numbered sequences in the range of SEQ ID NOs: 4-156, 158-280, and 284-366. In some embodiments, the polynucleotide has a nucleic acid sequence comprising at least 80%, 85%, 90%, 91%, 92%, 93%, 94%, 95%, 96%, 97%, 98%, 99% or more identity to the codon optimized nucleic acid sequences in the odd-numbered sequences in the range of SEQ ID NOs: 3-155, 157-279, and 283-365. In some embodiments, the codon optimized sequences of the odd-numbered sequences in the range of SEQ ID NOs: 3-155, 157-279, and 283-365, enhance expression of the encoded threonine aldolase or amino acid decarboxylase, providing preparations of enzyme capable of converting substrate to product.

In some embodiments, the polynucleotides are capable of hybridizing under highly stringent conditions to a reference sequence selected from the odd-numbered sequences in SEQ ID NOs: 3-155, 157-279, and 283-365, or a complement thereof, and encode a threonine aldolase or amino acid decarboxylase polypeptide.

In some embodiments, as described above, the polynucleotide encodes an engineered threonine aldolase or amino acid decarboxylase polypeptide with improved properties as compared to SEQ ID NO: 2, 158, or 282, wherein the polypeptide comprises an amino acid sequence having at least 80%, 85%, 86%, 87%, 88%, 89%, 90%, 91%, 92%, 93%, 94%, 95%, 96%, 97%, 98%, 99% or more identity to a reference sequence selected from SEQ ID NO: 2, 158, or 282, and one or more residue differences as compared to SEQ ID NO: 2, 158, or 282, wherein the sequence is selected from the even-numbered sequences in the range of SEQ ID NOs: 2-156, 158-280, and 284-366. In some embodiments, the reference amino acid sequence is selected from the even-numbered sequences in the range of SEQ ID NOs: 4-366. In some embodiments, the reference amino acid sequence is SEQ ID NO: 2, while in some other embodiments, the reference sequence is SEQ ID NO: 158, while in some other embodiments, the reference sequence is SEQ ID NO: 282.

In some embodiments, the polynucleotide encodes a threonine aldolase and/or amino acid decarboxylase polypeptide capable of converting one or more substrates to product with improved properties as compared to SEQ ID NO: 2, 158, or 282, wherein the polypeptide comprises an amino acid sequence having at least 80%, 85%, 86%, 87%, 88%, 89%, 90%, 91%, 92%, 93% 94%, 95% 96%, 97%, 98%, 99% or more sequence identity to reference sequence SEQ ID NO: 2, 158, or 282.

In some embodiments, the polynucleotide encoding the engineered threonine aldolase or amino acid decarboxylase comprises a polynucleotide sequence selected from the odd-numbered sequences in the range of SEQ ID NOs: 3-155, 157-279, and 283-365.

In some embodiments, the polynucleotides are capable of hybridizing under highly stringent conditions to a reference polynucleotide sequence selected from the odd-numbered sequences in the range of SEQ ID NOs: 3-365, or a complement thereof, and encode a threonine aldolase and/or amino acid decarboxylase polypeptide with one or more of the improved properties described herein. In some embodiments, the polynucleotide capable of hybridizing under highly stringent conditions encodes a threonine aldolase comprising an amino acid sequence having at least 80%, 85%, 86%, 87%, 88%, 89%, 90%, 91%, 92%, 93%, 94%, 95%, 96%, 97%, 98%, 99% or more identity to SEQ ID NO: 2, that has an amino acid sequence comprising one or more residue differences as compared to SEQ ID NO: 2, at residue positions selected from: 7/8/170/171, 7/170/171/172/195/196, 7/170/171/193, 8, 8/170/194/196, 10, 10/138/205/312, 10/167, 10/169/205, 10/198, 11/81/206, 11/206, 55/60, 55/60/171/174/253, 55/60/173, 55/60/247/250, 55/60/250, 55/60/250/253, 55/60/253, 55/170/253, 57/140/143/206, 60/247/253, 60/250/253, 60/253, 102, 138/167, 138/167/312, 138/169, 138/169/205, 138/169/276, 138/205, 138/245, 140/143/176/206, 140/143/206, 167/205, 169, 170, 170/171, 170/171/172/193/194, 170/172, 170/193/195/196, 170/196, 170/249, 170/253, 171, 171/172, 171/172/193/195/249, 171/174/201/253, 171/195, 171/201/253, 171/249, 171/253, 172, 173/247/250/324, 173/250/253, 174/211/253, 174/253, 191/253, 193, 193/194, 193/195, 194, 194/196, 195, 195/196, 196, 198/201/205, 201/205/246, 205, 206, 245, 249, 250/253, 253, and 276.

In some embodiments, the polynucleotides are capable of hybridizing under highly stringent conditions to a reference polynucleotide sequence selected from the odd-numbered sequences in the range of SEQ ID NOs: 3-365, or a complement thereof, and encode a threonine aldolase polypeptide with one or more of the improved properties described herein. In some embodiments, the polynucleotide capable of hybridizing under highly stringent conditions encodes a threonine aldolase polypeptide comprising an amino acid sequence having at least 80%, 85%, 86%, 87%, 88%, 89%, 90%, 91%, 92%, 93%, 94%, 95%, 96%, 97%, 98%, 99% or more identity to SEQ ID NO: 158, that has an amino acid sequence comprising one or more residue differences as compared to SEQ ID NO: 158, at residue positions selected from: 15/268/317, 128, 141/207/210/254/257/258, 141/207/210/254/258, 141/207/255/258, 141/211/212/254, 141/211/257/258, 141/212/254/257/258, 141/212/257/258, 141/254/255/257/258, 141/254/255/258, 141/254/257/258, 141/254/258, 141/255/257/258, 141/257/258, 141/258, 172/174/175, 172/174/175/200/203/205/245, 172/174/175/245, 172/174/175/245/248, 172/174/199/200/203/205/248, 172/174/245, 172/174/248, 172/175, 172/199/203/204/206, 174/175, 174/175/199/200/203/206/245/248, 174/175/199/200/205, 174/175/199/203/204/205, 174/175/245, 174/175/245/248, 174/245/248, 175/176/199, 175/245, 178/181/211/254/255/258, 178/207/211/254/255/257/258, 199/200/203/204/206/245, 199/200/203/206/248, 199/200/204, 199/200/206, 199/203/204, 199/203/204/206/245/248, 203/245/248, 207/210/211/255/257, 207/210/212/257, 207/210/255, 207/211, 207/211/212/254/255/257/258, 207/211/257/258, 207/212/255/258, 210/212/258, 210/254, 211/254/257/258, 211/254/258, 211/257/258, 245/248, 254/255/257/258, 254/255/258, 254/257, and 258.

In some embodiments, the polynucleotide capable of hybridizing under highly stringent conditions encodes an engineered amino acid decarboxylase with improved properties comprising an amino acid sequence having at least 80%, 85%, 86%, 87%, 88%, 89%, 90%, 91%, 92%, 93%, 94% 95%, 96%, 97%, 98%, 99% or more identity to SEQ ID NO: 282, and one or more residue differences as compared to SEQ ID NO: 282 at residue positions selected from: 48, 49, 55/74/110/121/194/233/253, 55/74/110/194/233/281/324, 55/74/110/211, 55/74/121/233/253/324, 55/74/194/233, 55/194/211/233/253, 55/211/253/324, 56, 63/398, 66/86/198/235/329, 66/198/202/290/316/329, 66/202/290/329, 66/290/329, 72, 74/110/121/194, 74/110/233/324, 74/110/324, 74/121/194/233/253, 74/121/194/233/324, 74/194, 74/194/233, 74/194/253, 80, 86, 86/162/186/187/202/203/235/329, 103, 135, 162, 162/187/202/235/252/290/316, 174, 183, 198, 245, 248, 258, 279, 280, and 350.

In some embodiments, the polynucleotide capable of hybridizing under highly stringent conditions encodes an engineered threonine aldolase or amino acid decarboxylase polypeptide with improved properties comprising an amino acid sequence having at least 80%, 85%, 86%, 87%, 88%, 89%, 90%, 91%, 92%, 93%, 94%, 95%, 96%, 97%, 98%, 99% or more identity to SEQ ID NO: 2, 158, or 282.

In some embodiments, the polynucleotides encode the polypeptides described herein but have at least about 80%, 85%, 86%, 87%, 88%, 89%, 90%, 91%, 92%, 93%, 94%, 95%, 96%, 97%, 98%, or 99% or more sequence identity at the nucleotide level to a reference polynucleotide encoding the engineered threonine aldolase or amino acid decarboxylase. In some embodiments, the reference polynucleotide sequence is selected from SEQ ID NOs: 3-365.

In some embodiments, the polynucleotide capable of hybridizing under highly stringent conditions encodes an engineered threonine aldolase polypeptide with improved properties comprising an amino acid sequence having at least 80%, 85%, 86%, 87%, 88%, 89%, 90%, 91%, 92%, 93%, 94%, 95%, 96%, 97%, 98%, 99% or more identity to SEQ ID NO: 2. In some embodiments, the polynucleotides encode the polypeptides described herein but have at least about 80%, 85%, 86%, 87%, 88%, 89%, 90%, 91%, 92%, 93%, 94%, 95%, 96%, 97%, 98%, or 99% or more sequence identity at the nucleotide level to a reference polynucleotide encoding the engineered threonine aldolase. In some embodiments, the reference polynucleotide sequence is selected from SEQ ID NOs: 3-155.

In some embodiments, the polynucleotide capable of hybridizing under highly stringent conditions encodes an engineered threonine aldolase polypeptide with improved properties comprising an amino acid sequence having at least 80%, 85%, 86%, 87%, 88%, 89%, 90%, 91%, 92%, 93%, 94%, 95%, 96%, 97%, 98%, 99% or more identity to SEQ ID NO: 158. In some embodiments, the polynucleotides encode the polypeptides described herein but have at least about 80%, 85%, 86%, 87%, 88%, 89%, 90%, 91%, 92%, 93%, 94%, 95%, 96%, 97%, 98%, or 99% or more sequence identity at the nucleotide level to a reference polynucleotide encoding the engineered threonine aldolase. In some embodiments, the reference polynucleotide sequence is selected from SEQ ID NOs: 159-281.

In some embodiments, the polynucleotide capable of hybridizing under highly stringent conditions encodes an engineered amino acid decarboxylase polypeptide having with improved properties comprising an amino acid sequence having at least 80%, 85%, 86%, 87%, 88%, 89%, 90%, 91%, 92%, 93%, 94%, 95%, 96%, 97%, 98%, 99% or more identity to SEQ ID NO: 282. In some embodiments, the polynucleotides encode the polypeptides described herein but have at least about 80%, 85%, 86%, 87%, 88%, 89%, 90%, 91%, 92%, 93%, 94%, 95%, 96%, 97%, 98%, or 99% or more sequence identity at the nucleotide level to a reference polynucleotide encoding the engineered amino acid decarboxylase. In some embodiments, the reference polynucleotide sequence is selected from SEQ ID NOs: 283-365.

In some embodiments, an isolated polynucleotide encoding any of the engineered threonine aldolase or amino acid decarboxylase polypeptides provided herein is manipulated in a variety of ways to provide for expression of the polypeptide. In some embodiments, the polynucleotides encoding the polypeptides are provided as expression vectors where one or more control sequences is present to regulate the expression of the polynucleotides and/or polypeptides. Manipulation of the isolated polynucleotide prior to its insertion into a vector may be desirable or necessary depending on the expression vector. The techniques for modifying polynucleotides and nucleic acid sequences utilizing recombinant DNA methods are well known in the art.

In some embodiments, the control sequences include among other sequences, promoters, leader sequences, polyadenylation sequences, propeptide sequences, signal peptide sequences, and transcription terminators. As known in the art, suitable promoters can be selected based on the host cells used. For bacterial host cells, suitable promoters for directing transcription of the nucleic acid constructs of the present application, include, but are not limited to the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroff et al., Proc. Natl Acad. Sci. USA 75: 3727-3731 [1978]), as well as the tac promoter (See e.g., DeBoer et al., Proc. Natl Acad. Sci. USA 80: 21-25 [1983]). Exemplary promoters for filamentous fungal host cells, include promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast 8:423-488 [1992]).

In some embodiments, the control sequence is a suitable transcription terminator sequence, a sequence recognized by a host cell to terminate transcription. The terminator sequence is operably linked to the 3′ terminus of the nucleic acid sequence encoding the polypeptide. Any terminator which is functional in the host cell of choice finds use in the present invention. For example, exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra).

In some embodiments, the control sequence is a suitable leader sequence, anon-translated region of an mRNA that is important for translation by the host cell. The leader sequence is operably linked to the 5′ terminus of the nucleic acid sequence encoding the polypeptide. Any leader sequence that is functional in the host cell of choice may be used. Exemplary leaders for filamentous fungal host cells are obtained from the genes for Aspergillus oryzae TAKA amylase and Aspergillus nidulans triose phosphate isomerase. Suitable leaders for yeast host cells include, but are not limited to those obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). The control sequence may also be a polyadenylation sequence, a sequence operably linked to the 3′ terminus of the nucleic acid sequence and which, when transcribed, is recognized by the host cell as a signal to add polyadenosine residues to transcribed mRNA. Any polyadenylation sequence which is functional in the host cell of choice may be used in the present invention. Exemplary polyadenylation sequences for filamentous fungal host cells include, but are not limited to those from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are also known in the art (See e.g., Guo and Sherman, Mol. Cell. Bio., 15:5983-5990 [1995]).

In some embodiments, the control sequence is a signal peptide coding region that codes for an amino acid sequence linked to the amino terminus of a polypeptide and directs the encoded polypeptide into the cell's secretory pathway. The 5′ end of the coding sequence of the nucleic acid sequence may inherently contain a signal peptide coding region naturally linked in translation reading frame with the segment of the coding region that encodes the secreted polypeptide. Alternatively, the 5′ end of the coding sequence may contain a signal peptide coding region that is foreign to the coding sequence. Any signal peptide coding region that directs the expressed polypeptide into the secretory pathway of a host cell of choice finds use for expression of the engineered threonine aldolase or amino acid decarboxylase polypeptides provided herein. Effective signal peptide coding regions for bacterial host cells include, but are not limited to the signal peptide coding regions obtained from the genes for Bacillus NC1B 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (See e.g., Simonen and Palva, Microbiol. Rev., 57:109-137 [1993]). Effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase.

In some embodiments, the control sequence is a propeptide coding region that codes for an amino acid sequence positioned at the amino terminus of a polypeptide. The resultant polypeptide is referred to as a “proenzyme,” “propolypeptide,” or “zymogen,” in some cases). A propolypeptide can be converted to a mature active polypeptide by catalytic or autocatalytic cleavage of the propeptide from the propolypeptide. The propeptide coding region includes, but is not limited to the genes for Bacillus subtilis alkaline protease (aprE), Bacillus subtilis neutral protease (nprT), Saccharomyces cerevisiae alpha-factor, Rhizomucor miehei aspartic proteinase, and Myceliophthora thermophila lactase (See e.g., WO 95/33836). Where both signal peptide and propeptide regions are present at the amino terminus of a polypeptide, the propeptide region is positioned next to the amino terminus of a polypeptide and the signal peptide region is positioned next to the amino terminus of the propeptide region.

In some embodiments, regulatory sequences are also utilized. These sequences facilitate the regulation of the expression of the polypeptide relative to the growth of the host cell. Examples of regulatory systems are those which cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter.

The present invention also provides recombinant expression vectors comprising a polynucleotide encoding an engineered threonine aldolase or amino acid decarboxylase polypeptide, and one or more expression regulating regions such as a promoter and a terminator, a replication origin, etc., depending on the type of hosts into which they are to be introduced. In some embodiments, the various nucleic acid and control sequences described above are combined together to produce a recombinant expression vector which includes one or more convenient restriction sites to allow for insertion or substitution of the nucleic acid sequence encoding the variant threonine aldolase or amino acid decarboxylase polypeptide at such sites. Alternatively, the polynucleotide sequence(s) of the present invention are expressed by inserting the polynucleotide sequence or a nucleic acid construct comprising the polynucleotide sequence into an appropriate vector for expression. In creating the expression vector, the coding sequence is located in the vector so that the coding sequence is operably linked with the appropriate control sequences for expression.

The recombinant expression vector may be any vector (e.g., a plasmid or virus), that can be conveniently subjected to recombinant DNA procedures and can result in the expression of the variant threonine aldolase and amino acid decarboxylase polynucleotide sequence. The choice of the vector will typically depend on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

In some embodiments, the expression vector preferably contains one or more selectable markers, which permit easy selection of transformed cells. A “selectable marker” is a gene the product of which provides for biocide or viral resistance, resistance to heavy metals, prototrophy to auxotrophy, and the like. Examples of bacterial selectable markers include, but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in a filamentous fungal host cell include, but are not limited to, amdS (acetamidase), argB (ornithine carbamoyltransferases), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. In another aspect, the present invention provides a host cell comprising a polynucleotide encoding at least one engineered threonine aldolase and amino acid decarboxylase polypeptide of the present invention, the polynucleotide being operatively linked to one or more control sequences for expression of the engineered threonine aldolase and amino acid decarboxylase enzyme(s) in the host cell. Host cells for use in expressing the polypeptides encoded by the expression vectors of the present invention are well known in the art and include but are not limited to, bacterial cells, such as E. coli, Vibrio fluvialis, Streptomyces and Salmonella typhimurium cells; fungal cells, such as yeast cells (e.g., Saccharomyces cerevisiae and Pichia pastoris [ATCC Accession No. 201178]); insect cells such as Drosophila S2 and Spodoptera Sf9 cells; animal cells such as CHO, COS, BHK, 293, and Bowes melanoma cells; and plant cells. Exemplary host cells are Escherichia coli strains (e.g., W3110 (ΔfhuA) and BL21).

Accordingly, in another aspect, the present invention provides methods for producing the engineered threonine aldolase and amino acid decarboxylase polypeptides, where the methods comprise culturing a host cell capable of expressing a polynucleotide encoding the engineered threonine aldolase and amino acid decarboxylase polypeptide under conditions suitable for expression of the polypeptide. In some embodiments, the methods further comprise the steps of isolating and/or purifying the threonine aldolase and amino acid decarboxylase polypeptides, as described herein.

Appropriate culture media and growth conditions for the above-described host cells are well known in the art. Polynucleotides for expression of the threonine aldolase and amino acid decarboxylase polypeptides may be introduced into cells by various methods known in the art. Techniques include, among others, electroporation, biolistic particle bombardment, liposome mediated transfection, calcium chloride transfection, and protoplast fusion.

The engineered threonine aldolases and amino acid decarboxylases with the properties disclosed herein can be obtained by subjecting the polynucleotide encoding the naturally occurring or engineered threonine aldolase and amino acid decarboxylase polypeptide to mutagenesis and/or directed evolution methods known in the art, and as described herein. An exemplary directed evolution technique is mutagenesis and/or DNA shuffling (See e.g., Stemmer, Proc. Natl. Acad. Sci. USA 91:10747-10751 [1994]; WO 95/22625; WO 97/0078; WO 97/35966; WO 98/27230; WO 00/42651; WO 01/75767 and U.S. Pat. No. 6,537,746). Other directed evolution procedures that can be used include, among others, staggered extension process (StEP), in vitro recombination (See e.g., Zhao et al., Nat. Biotechnol., 16:258-261 [1998]), mutagenic PCR (See e.g., Caldwell et al., PCR Methods Appl., 3:S136-S140 [1994]), and cassette mutagenesis (See e.g., Black et al., Proc. Natl. Acad. Sci. USA 93:3525-3529 [1996]).

For example, mutagenesis and directed evolution methods can be readily applied to polynucleotides to generate variant libraries that can be expressed, screened, and assayed. Mutagenesis and directed evolution methods are well known in the art (See e.g., U.S. Pat. Nos. 5,605,793, 5,811,238, 5,830,721, 5,834,252, 5,837,458, 5,928,905, 6,096,548, 6,117,679, 6,132,970, 6,165,793, 6,180,406, 6,251,674, 6,265,201, 6,277,638, 6,287,861, 6,287,862, 6,291,242, 6,297,053, 6,303,344, 6,309,883, 6,319,713, 6,319,714, 6,323,030, 6,326,204, 6,335,160, 6,335,198, 6,344,356, 6,352,859, 6,355,484, 6,358,740, 6,358,742, 6,365,377, 6,365,408, 6,368,861, 6,372,497, 6,337,186, 6,376,246, 6,379,964, 6,387,702, 6,391,552, 6,391,640, 6,395,547, 6,406,855, 6,406,910, 6,413,745, 6,413,774, 6,420,175, 6,423,542, 6,426,224, 6,436,675, 6,444,468, 6,455,253, 6,479,652, 6,482,647, 6,483,011, 6,484,105, 6,489,146, 6,500,617, 6,500,639, 6,506,602, 6,506,603, 6,518,065, 6,519,065, 6,521,453, 6,528,311, 6,537,746, 6,573,098, 6,576,467, 6,579,678, 6,586,182, 6,602,986, 6,605,430, 6,613,514, 6,653,072, 6,686,515, 6,703,240, 6,716,631, 6,825,001, 6,902,922, 6,917,882, 6,946,296, 6,961,664, 6,995,017, 7,024,312, 7,058,515, 7,105,297, 7,148,054, 7,220,566, 7,288,375, 7,384,387, 7,421,347, 7,430,477, 7,462,469, 7,534,564, 7,620,500, 7,620,502, 7,629,170, 7,702,464, 7,747,391, 7,747,393, 7,751,986, 7,776,598, 7,783,428, 7,795,030, 7,853,410, 7,868,138, 7,783,428, 7,873,477, 7,873,499, 7,904,249, 7,957,912, 7,981,614, 8,014,961, 8,029,988, 8,048,674, 8,058,001, 8,076,138, 8,108,150, 8,170,806, 8,224,580, 8,377,681, 8,383,346, 8,457,903, 8,504,498, 8,589,085, 8,762,066, 8,768,871, 9,593,326, and all related US, as well as PCT and non-US counterparts; Ling et al., Anal. Biochem., 254(2):157-78 [1997]; Dale et al., Meth. Mol. Biol., 57:369-74 [1996]; Smith, Ann. Rev. Genet., 19:423-462 [1985]; Botstein et al., Science, 229:1193-1201 [1985]; Carter, Biochem. J., 237:1-7 [1986]; Kramer et al., Cell, 38:879-887 [1984]; Wells et al., Gene, 34:315-323 [1985]; Minshull et al., Curr. Op. Chem. Biol., 3:284-290 [1999]; Christians et al., Nat. Biotechnol., 17:259-264 [1999]; Crameri et al., Nature, 391:288-291 [1998]; Crameri, et al., Nat. Biotechnol., 15:436-438 [1997]; Zhang et al., Proc. Nat. Acad. Sci. U.S.A., 94:4504-4509 [1997]; Crameri et al., Nat. Biotechnol., 14:315-319 [1996]; Stemmer, Nature, 370:389-391 [1994]; Stemmer, Proc. Nat. Acad. Sci. USA, 91:10747-10751 [1994]; WO 95/22625; WO 97/0078; WO 97/35966; WO 98/27230; WO 00/42651; WO 01/75767; and WO 2009/152336, all of which are incorporated herein by reference).

In some embodiments, the enzyme clones obtained following mutagenesis treatment are screened by subjecting the enzymes to a defined temperature (or other assay conditions, such as testing the enzyme's activity over a broad range of substrates) and measuring the amount of enzyme activity remaining after heat treatments or other assay conditions. Clones containing a polynucleotide encoding a threonine aldolase or amino acid decarboxylase polypeptide are then sequenced to identify the nucleotide sequence changes (if any), and used to express the enzyme in a host cell. Measuring enzyme activity from the expression libraries can be performed using any suitable method known in the art (e.g., standard biochemistry techniques, such as HPLC analysis).

In some embodiments, the clones obtained following mutagenesis treatment can be screened for engineered threonine aldolases or amino acid decarboxylases having one or more desired improved enzyme properties (e.g., improved regioselectivity). Measuring enzyme activity from the expression libraries can be performed using the standard biochemistry techniques, such as GC analysis, HPLC analysis and/or derivatization of products (pre or post separation), for example, using dansyl chloride or OPA (See e.g., Yaegaki et al., J Chromatogr. 356(1):163-70 [1986]).

When the sequence of the engineered polypeptide is known, the polynucleotides encoding the enzyme can be prepared by standard solid-phase methods, according to known synthetic methods. In some embodiments, fragments of up to about 100 bases can be individually synthesized, then joined (e.g., by enzymatic or chemical ligation methods, or polymerase mediated methods) to form any desired continuous sequence. For example, polynucleotides and oligonucleotides encoding portions of the threonine aldolase or amino acid decarboxylase can be prepared by chemical synthesis as known in the art (e.g., the classical phosphoramidite method of Beaucage et al., Tet. Lett. 22:1859-69 [1981], or the method described by Matthes et al., EMBO J. 3:801-05 [1984]) as typically practiced in automated synthetic methods. According to the phosphoramidite method, oligonucleotides are synthesized (e.g., in an automatic DNA synthesizer), purified, annealed, ligated and cloned in appropriate vectors. In addition, essentially any nucleic acid can be obtained from any of a variety of commercial sources. In some embodiments, additional variations can be created by synthesizing oligonucleotides containing deletions, insertions, and/or substitutions, and combining the oligonucleotides in various permutations to create engineered threonine aldolases or amino acid decarboxylases with improved properties.

Accordingly, in some embodiments, a method for preparing the engineered threonine aldolase polypeptide comprises: (a) synthesizing a polynucleotide encoding a polypeptide comprising an amino acid sequence having at least about 80%, 85%, 86%, 87%, 88%, 89%, 90%, 91%, 92%, 93%, 94%, 95%, 96%, 97%, 98%, or 99% or more sequence identity to an amino acid sequence selected from the even-numbered sequences of SEQ ID NOs: 4-280, and having one or more residue differences as compared to SEQ ID NO: 2 or 158; and (b) expressing the threonine aldolase polypeptide encoded by the polynucleotide.

Accordingly, in some embodiments, a method for preparing the engineered amino acid decarboxylase polypeptide comprises: (a) synthesizing a polynucleotide encoding a polypeptide comprising an amino acid sequence having at least about 80%, 85%, 86%, 87%, 88%, 89%, 90%, 91%, 92%, 93%, 94%, 95%, 96%, 97%, 98%, or 99% or more sequence identity to an amino acid sequence selected from the even-numbered sequences of SEQ ID NOs: 282-366, and having one or more residue differences as compared to SEQ ID NO: 282; and (b) expressing the amino acid decarboxylase polypeptide encoded by the polynucleotide.

In some embodiments of the method, the polynucleotide encodes an engineered threonine aldolase or amino acid decarboxylase that has optionally one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions can be conservative or non-conservative substitutions.

In some embodiments, any of the engineered threonine aldolase or amino acid decarboxylase enzymes expressed in a host cell can be recovered from the cells and/or the culture medium using any one or more of the well-known techniques for protein purification, including, among others, lysozyme treatment, sonication, filtration, salting-out, ultra-centrifugation, and chromatography. Suitable solutions for lysing and the high efficiency extraction of proteins from bacteria, such as E. coli, are commercially available (e.g., CelLytic B™, Sigma-Aldrich, St. Louis MO).

Chromatographic techniques for isolation of the threonine aldolase or amino acid decarboxylase polypeptide include, among others, reverse phase chromatography high performance liquid chromatography, ion exchange chromatography, gel electrophoresis, and affinity chromatography. Conditions for purifying a particular enzyme will depend, in part, on factors such as net charge, hydrophobicity, hydrophilicity, molecular weight, molecular shape, etc., and will be apparent to those having skill in the art.

In some embodiments, affinity techniques may be used to isolate the improved threonine aldolase or amino acid decarboxylase enzymes. For affinity chromatography purification, any antibody which specifically binds the threonine aldolase or amino acid decarboxylase polypeptide may be used. For the production of antibodies, various host animals, including but not limited to rabbits, mice, rats, etc., may be immunized by injection with a threonine aldolase or amino acid decarboxylase polypeptide, or a fragment thereof. The threonine aldolase or amino acid decarboxylase polypeptide or fragment may be attached to a suitable carrier, such as BSA, by means of a side chain functional group or linkers attached to a side chain functional group. In some embodiments, the affinity purification can use a specific ligand bound by the threonine aldolase or amino acid decarboxylase or dye affinity column (See e.g., EP0641862; Stellwagen, “Dye Affinity Chromatography,” In Current Protocols in Protein Science, Unit 9.2-9.2.16 [2001]).

### Methods of Using the Engineered Threonine Aldolase and Amino Acid Decarboxylase Enzymes

In some embodiments, the threonine aldolase or amino acid decarboxylase enzymes described herein find use in processes for conversion of one or more suitable substrates to a product.

In another aspect, the engineered threonine aldolase or amino acid decarboxylase polypeptides disclosed herein can be used in a process for the conversion of the substrate compound (1), or structural analogs thereof, and of the substrate compound (2), or structural analogs thereof, to the product of compound (3) or the corresponding structural analog and/or to the product of compound (4) or the corresponding structural analog.

Structural analogs of compound (1) include other ketones with halogen modifications and/or ketones with various modifications at the alpha carbons. Structural analogs of compound (2) include other small amino acids or amino acid analogs. Structural analogs of compound (3) include various β-hydroxy-α-amino acids. Structural analogs of compound (4) include other amino alcohols.

In some embodiments, the present disclosure provides a process of preparing compound (4);

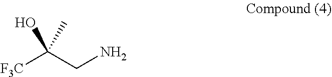

the process comprising a step of contacting a substrate of compound (3)

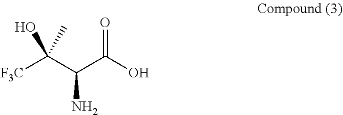

with an engineered amino acid decarboxylase polypeptide as disclosed herein under suitable reaction conditions, such that a product of compound (4) is prepared.

In some embodiments, the present disclosure provides a process of preparing compound (3);

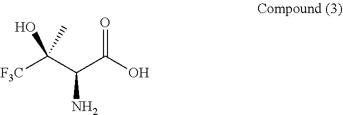

the process comprising a step of contacting a substrate of compound (1)

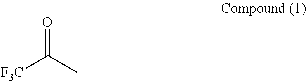

with a substrate of compound (2)

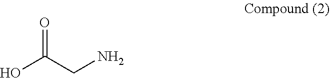

with an engineered threonine aldolase polypeptide as disclosed herein under suitable reaction conditions, such that a product of compound (3) is prepared.

In the embodiments provided herein and illustrated in the Examples, various ranges of suitable reaction conditions that can be used in the processes, include but are not limited to, substrate loading, co-substrate loading, pH, temperature, buffer, solvent system, polypeptide loading, and reaction time. Further suitable reaction conditions for carrying out the process for biocatalytic conversion of substrate compounds to product compounds using an engineered threonine aldolase or amino acid decarboxylase described herein can be readily optimized in view of the guidance provided herein by routine experimentation that includes, but is not limited to, contacting the engineered threonine aldolase or amino acid decarboxylase polypeptide and one or more substrate compounds under experimental reaction conditions of concentration, pH, temperature, and solvent conditions, and detecting the product compound.

The substrate compound(s) in the reaction mixtures can be varied, taking into consideration, for example, the desired amount of product compound, the effect of each substrate concentration on enzyme activity, stability of enzyme under reaction conditions, and the percent conversion of each substrate to product. In some embodiments, the suitable reaction conditions comprise a substrate compound loading for each of one of more substrates of at least about 0.5 to about 25 g/L, 1 to about 25 g/L, 5 to about 25 g/L, about 10 to about 25 g/L, or 20 to about 25 g/L. In some embodiments, the suitable reaction conditions comprise a substrate compound loading for each of one of more substrates of at least about 0.5 g/L, at least about 1 g/L, at least about 5 g/L, at least about 10 g/L, at least about 15 g/L, at least about 20 g/L, or at least about 30 g/L, or even greater.

In carrying out the threonine aldolase and amino acid decarboxylase mediated processes described herein, the engineered polypeptide may be added to the reaction mixture in the form of a purified enzyme, partially purified enzyme, whole cells transformed with gene(s) encoding the enzyme, as cell extracts and/or lysates of such cells, and/or as an enzyme immobilized on a solid support. Whole cells transformed with gene(s) encoding the engineered threonine aldolase or amino acid decarboxylase enzyme or cell extracts, lysates thereof, and isolated enzymes may be employed in a variety of different forms, including solid (e.g., lyophilized, spray-dried, and the like) or semisolid (e.g., a crude paste). The cell extracts or cell lysates may be partially purified by precipitation (ammonium sulfate, polyethyleneimine, heat treatment or the like, followed by a desalting procedure prior to lyophilization (e.g., ultrafiltration, dialysis, etc.). Any of the enzyme preparations (including whole cell preparations) may be stabilized by crosslinking using known crosslinking agents, such as, for example, glutaraldehyde or immobilization to a solid phase (e.g., Eupergit C, and the like).

The gene(s) encoding the engineered threonine aldolase or amino acid decarboxylase polypeptides can be transformed into host cell separately or together into the same host cell. For example, in some embodiments one set of host cells can be transformed with gene(s) encoding one engineered threonine aldolase or amino acid decarboxylase polypeptide and another set can be transformed with gene(s) encoding another engineered threonine aldolase or amino acid decarboxylase polypeptide. Both sets of transformed cells can be utilized together in the reaction mixture in the form of whole cells, or in the form of lysates or extracts derived therefrom. In other embodiments, a host cell can be transformed with gene(s) encoding multiple engineered threonine aldolase or amino acid decarboxylase polypeptides. In some embodiments the engineered polypeptides can be expressed in the form of secreted polypeptides and the culture medium containing the secreted polypeptides can be used for the threonine aldolase or amino acid decarboxylase reaction.

In some embodiments, the improved activity and/or regioselectivity and/or stereoselectivity of the engineered threonine aldolase or amino acid decarboxylase polypeptides disclosed herein provides for processes wherein higher percentage conversion can be achieved with lower concentrations of the engineered polypeptide. In some embodiments of the process, the suitable reaction conditions comprise an engineered polypeptide amount of about 1% (w/w), 2% (w/w), 5% (w/w), 10% (w/w), 20% (w/w), 30% (w/w), 40% (w/w), 50% (w/w), 75% (w/w), 100% (w/w) or more of substrate compound loading.

In some embodiments, the engineered polypeptide is present at about 0.01 g/L to about 50 g/L; about 0.05 g/L to about 50 g/L; about 0.1 g/L to about 40 g/L; about 1 g/L to about 40 g/L; about 2 g/L to about 40 g/L; about 5 g/L to about 40 g/L; about 5 g/L to about 30 g/L; about 0.1 g/L to about 10 g/L; about 0.5 g/L to about 10 g/L; about 1 g/L to about 10 g/L; about 0.1 g/L to about 5 g/L; about 0.5 g/L to about 5 g/L; or about 0.1 g/L to about 2 g/L. In some embodiments, the threonine aldolase or amino acid decarboxylase polypeptide is present at about 0.01 g/L, 0.05 g/L, 0.1 g/L, 0.2 g/L, 0.5 g/L, 1, 2 g/L, 5 g/L, 10 g/L, 15 g/L, 20 g/L, 25 g/L, 30 g/L, 35 g/L, 40 g/L, or 50 g/L.

During the course of the reaction, the pH of the reaction mixture may change. The pH of the reaction mixture may be maintained at a desired pH or within a desired pH range. This may be done by the addition of an acid or a base, before and/or during the course of the reaction. Alternatively, the pH may be controlled by using a buffer. Accordingly, in some embodiments, the reaction condition comprises a buffer. Suitable buffers to maintain desired pH ranges are known in the art and include, by way of example and not limitation, borate, phosphate, 2-(N-morpholino)ethanesulfonic acid (MES), 3-(N-morpholino)propanesulfonic acid (MOPS), acetate, triethanolamine, and 2-amino-2-hydroxymethyl-propane-1,3-diol (Tris), and the like. In some embodiments, the reaction conditions comprise water as a suitable solvent with no buffer present.

In the embodiments of the process, the reaction conditions comprise a suitable pH. The desired pH or desired pH range can be maintained by use of an acid or base, an appropriate buffer, or a combination of buffering and acid or base addition. The pH of the reaction mixture can be controlled before and/or during the course of the reaction. In some embodiments, the suitable reaction conditions comprise a solution pH from about 4 to about 10, pH from about 5 to about 10, pH from about 5 to about 9, pH from about 6 to about 9, pH from about 6 to about 8. In some embodiments, the reaction conditions comprise a solution pH of about 4, 4.5, 5, 5.5, 6, 6.5, 7, 7.5, 8, 8.5, 9, 9.5, or 10.

In the embodiments of the processes herein, a suitable temperature is used for the reaction conditions, for example, taking into consideration the increase in reaction rate at higher temperatures, and the activity of the enzyme during the reaction time period. Accordingly, in some embodiments, the suitable reaction conditions comprise a temperature of about 10° C. to about 60° C., about 10° C. to about 55° C., about 15° C. to about 60° C., about 20° C. to about 60° C., about 20° C. to about 55° C., about 25° C. to about 55° C., or about 30° C. to about 50° C. In some embodiments, the suitable reaction conditions comprise a temperature of about 10° C., 15° C., 20° C., 25° C., 30° C., 35° C., 40° C., 45° C., 50° C., 55° C., or 60° C. In some embodiments, the temperature during the enzymatic reaction can be maintained at a specific temperature throughout the course of the reaction. In some embodiments, the temperature during the enzymatic reaction can be adjusted over a temperature profile during the course of the reaction.

In some embodiments, the processes of the invention are carried out in a solvent. Suitable solvents include water, aqueous buffer solutions, organic solvents, polymeric solvents, and/or co-solvent systems, which generally comprise aqueous solvents, organic solvents and/or polymeric solvents. The aqueous solvent (water or aqueous co-solvent system) may be pH-buffered or unbuffered. In some embodiments, the processes using the engineered threonine aldolase or amino acid decarboxylase polypeptides can be carried out in an aqueous co-solvent system comprising an organic solvent (e.g., ethanol, isopropanol (IPA), dimethyl sulfoxide (DMSO), dimethylformamide (DMF) ethyl acetate, butyl acetate, 1-octanol, heptane, octane, methyl tert-butyl ether (MTBE), toluene, and the like), ionic or polar solvents (e.g., 1-ethyl-4-methylimidazolium tetrafluoroborate, 1-butyl-3-methylimidazolium tetrafluoroborate, 1-butyl-3-methylimidazolium hexafluorophosphate, glycerol, polyethylene glycol, and the like). In some embodiments, the co-solvent can be a polar solvent, such as a polyol, dimethylsulfoxide (DMSO), or lower alcohol. The non-aqueous co-solvent component of an aqueous co-solvent system may be miscible with the aqueous component, providing a single liquid phase, or may be partly miscible or immiscible with the aqueous component, providing two liquid phases. Exemplary aqueous co-solvent systems can comprise water and one or more co-solvents selected from an organic solvent, polar solvent, and polyol solvent. In general, the co-solvent component of an aqueous co-solvent system is chosen such that it does not adversely inactivate the threonine aldolase or amino acid decarboxylase enzyme under the reaction conditions. Appropriate co-solvent systems can be readily identified by measuring the enzymatic activity of the specified engineered threonine aldolase or amino acid decarboxylase enzyme with a defined substrate of interest in the candidate solvent system, utilizing an enzyme activity assay, such as those described herein.

In some embodiments of the process, the suitable reaction conditions comprise an aqueous co-solvent, where the co-solvent comprises DMSO at about 1% to about 50% (v/v), about 1 to about 40% (v/v), about 2% to about 40% (v/v), about 5% to about 30% (v/v), about 10% to about 30% (v/v), or about 10% to about 20% (v/v). In some embodiments of the process, the suitable reaction conditions can comprise an aqueous co-solvent comprising ethanol at about 1% (v/v), about 5% (v/v), about 10% (v/v), about 15% (v/v), about 20% (v/v), about 25% (v/v), about 30% (v/v), about 35% (v/v), about 40% (v/v), about 45% (v/v), or about 50% (v/v).

In some embodiments, the reaction conditions comprise a surfactant for stabilizing or enhancing the reaction. Surfactants can comprise non-ionic, cationic, anionic and/or amphiphilic surfactants. Exemplary surfactants, include by way of example and not limitation, nonyl phenoxypolyethoxylethanol (NP40), TRITON™ X-100 polyethylene glycol tert-octylphenyl ether, polyoxyethylene-stearylamine, cetyltrimethylammonium bromide, sodium oleylamidosulfate, polyoxyethylene-sorbitanmonostearate, hexadecyldimethylamine, etc. Any surfactant that may stabilize or enhance the reaction may be employed. The concentration of the surfactant to be employed in the reaction may be generally from 0.1 to 50 mg/ml, particularly from 1 to 20 mg/ml.

In some embodiments, the reaction conditions include an antifoam agent, which aids in reducing or preventing formation of foam in the reaction solution, such as when the reaction solutions are mixed or sparged. Anti-foam agents include non-polar oils (e.g., minerals, silicones, etc.), polar oils (e.g., fatty acids, alkyl amines, alkyl amides, alkyl sulfates, etc.), and hydrophobic (e.g., treated silica, polypropylene, etc.), some of which also function as surfactants. Exemplary anti-foam agents include, Y-30® (Dow Corning), poly-glycol copolymers, oxy/ethoxylated alcohols, and polydimethylsiloxanes. In some embodiments, the anti-foam can be present at about 0.001% (v/v) to about 5% (v/v), about 0.01% (v/v) to about 5% (v/v), about 0.1% (v/v) to about 5% (v/v), or about 0.1% (v/v) to about 2% (v/v). In some embodiments, the anti-foam agent can be present at about 0.001% (v/v), about 0.01% (v/v), about 0.1% (v/v), about 0.5% (v/v), about 1% (v/v), about 2% (v/v), about 3% (v/v), about 4% (v/v), or about 5% (v/v) or more as desirable to promote the reaction.

The quantities of reactants used in the threonine aldolase or amino acid decarboxylase reaction will generally vary depending on the quantities of product desired, and concomitantly the amount of threonine aldolase or amino acid decarboxylase substrate employed. Those having ordinary skill in the art will readily understand how to vary these quantities to tailor them to the desired level of productivity and scale of production.

In some embodiments, the order of addition of reactants is not critical. The reactants may be added together at the same time to a solvent (e.g., monophasic solvent, biphasic aqueous co-solvent system, and the like), or alternatively, some of the reactants may be added separately, and some together at different time points. For example, the cofactor, co-substrate and substrate may be added first to the solvent.

The solid reactants (e.g., enzyme, salts, etc.) may be provided to the reaction in a variety of different forms, including powder (e.g., lyophilized, spray dried, and the like), solution, emulsion, suspension, and the like. The reactants can be readily lyophilized or spray dried using methods and equipment that are known to those having ordinary skill in the art. For example, the protein solution can be frozen at −80° C. in small aliquots, then added to a pre-chilled lyophilization chamber, followed by the application of a vacuum.

For improved mixing efficiency when an aqueous co-solvent system is used, the threonine aldolase or amino acid decarboxylase, and co-substrate may be added and mixed into the aqueous phase first. The threonine aldolase or amino acid decarboxylase substrate may be added and mixed in, followed by the organic phase or the substrate may be dissolved in the organic phase and mixed in. Alternatively, the threonine aldolase or amino acid decarboxylase substrate may be premixed in the organic phase, prior to addition to the aqueous phase.

The processes of the present invention are generally allowed to proceed until further conversion of substrate to product does not change significantly with reaction time (e.g., less than 10% of substrate being converted, or less than 5% of substrate being converted). In some embodiments, the reaction is allowed to proceed until there is complete or near complete conversion of substrate to product. Transformation of substrate to product can be monitored using known methods by detecting substrate and/or product, with or without derivatization. Suitable analytical methods include gas chromatography, HPLC, MS, and the like.

In some embodiments of the process, the suitable reaction conditions comprise a substrate loading for each of one or more substrates of at least about 5 g/L, 10 g/L, 20 g/L, or more, and wherein the method results in at least about 50%, 60%, 70%, 80%, 90%, 95% or greater conversion of substrate compound to product compound in about in about 24 h or less, in about 12 h or less, in about 6 h or less, or in about 4 h or less.

The engineered threonine aldolase or amino acid decarboxylase polypeptides of the present invention when used in the process under suitable reaction conditions result in an excess of the desired product in at least 30%, 40%, 50%, 60%, or greater enantiomeric excess over undesired product(s).

In some further embodiments of the processes for converting one or more substrate compounds to product compound using the engineered threonine aldolase or amino acid decarboxylase polypeptides, the suitable reaction conditions can comprise an initial substrate loading for each of one or more substrates to the reaction solution which is then contacted by the polypeptide. This reaction solution is then further supplemented with additional substrate compound as a continuous or batchwise addition over time at a rate of at least about 1 g/L/h, at least about 2 g/L/h, at least about 4 g/L/h, at least about 6 g/L/h, or higher for each of one or more substrate compounds. Thus, according to these suitable reaction conditions, polypeptide is added to a solution having an initial substrate loading of at least about 1 g/L, 5 g/L, or 10 g/L for each of one or more substrate compounds. This addition of polypeptide is then followed by continuous addition of further substrate to the solution at a rate of about 2 g/L/h, 4 g/L/h, or 6 g/L/h for each of one or more substrate compounds until a much higher final substrate loading of at least about 30 g/L or more for each of one or more substrate compounds, is reached. Accordingly, in some embodiments of the process, the suitable reaction conditions comprise addition of the polypeptide to a solution having an initial substrate loading of at least about 1 g/L, 5 g/L, or 10 g/L followed by addition of further substrate to the solution at a rate of about 2 g/L/h, 4 g/L/h, or 6 g/L/h until a final substrate loading of at least about 30 g/L, or more, is reached for each of one or more substrate compounds. This substrate supplementation reaction condition allows for higher substrate loadings to be achieved while maintaining high rates of conversion of substrate to product of at least about 5%, 25%, 50%, 75%, 90% or greater conversion of substrate for either or both of one or more substrate compounds.

Any of the processes disclosed herein using the engineered polypeptides for the preparation of compound (4) and/or compound (3) can be carried out under a range of suitable reaction conditions, including but not limited to ranges of ketone substrates, ranges of amino acid substrates, temperature, pH, solvent system, substrate loading, polypeptide loading, cofactor loading, and reaction time. In one example, in some embodiments, the preparation of compound (4) and/or compound (3) can be carried out wherein the suitable reaction conditions comprise: (a) ketone substrate loading of about 0.01 M to 1 M of substrate compound; (b) amino acid substrate loading of about 0.01 M to 1 M of substrate compound; (c) of about 0.5 g/L to 100 g/L of each engineered polypeptide; (d) 0.01 M-1 M triethanolamine-HCl buffer; (e) 0.1-2.0 g/L PLP; (f) pH at 6-9; and (g) temperature of about 20° C. to 60° C. In some embodiments, the suitable reaction conditions comprise: (a) about 0.1 M of compound (1) substrate compound); (b) about 0.1 M of compound (2) substrate compound); (c) about 50 g/L of each engineered polypeptide; (d) 0.1 M triethanolamine-HCl buffer; (e) 0.75 g/L of PLP; (f) static pH at 7, and (g) about 30° C.

In some embodiments, additional reaction components or additional techniques carried out to supplement the reaction conditions. These can include taking measures to stabilize or prevent inactivation of the enzyme, reduce product inhibition, shift reaction equilibrium to formation of the desired product.

In further embodiments, any of the above described process for the conversion of one or more substrate compounds to product compound can further comprise one or more steps selected from: extraction; isolation; purification; and crystallization of product compound. Methods, techniques, and protocols for extracting, isolating, purifying, and/or crystallizing the product from biocatalytic reaction mixtures produced by the above disclosed processes are known to the ordinary artisan and/or accessed through routine experimentation. Additionally, illustrative methods are provided in the Examples below.

Various features and embodiments of the invention are illustrated in the following representative examples, which are intended to be illustrative, and not limiting.

### EXPERIMENTAL

The following Examples, including experiments and results achieved, are provided for illustrative purposes only and are not to be construed as limiting the present invention.

In the Examples below, the following abbreviations apply: ppm (parts per million); M (molar); mM (millimolar), uM and μM (micromolar); nM (nanomolar); mol (moles); gm and g (gram); mg (milligrams); ug and μg (micrograms); L and l (liter); ml and mL (milliliter); cm (centimeters); mm (millimeters); um and μm (micrometers); sec. (seconds); min(s) (minute(s)); h(s) and hr(s) (hour(s)); U (units); MW (molecular weight); rpm (rotations per minute); psi and PSI (pounds per square inch); ° C. (degrees Centigrade); RT and rt (room temperature); CAM and cam (chloramphenicol); DMSO (dimethylsulfoxide); PMBS (polymyxin B sulfate); IPTG (isopropyl β-D-1-thiogalactopyranoside); LB (Luria-Bertani broth); TB (Terrific Broth; 12 g/L bacto-tryptone, 24 g/L yeast extract, 4 mL/L glycerol, 65 mM potassium phosphate, pH 7.0, 1 mM MgSO₄); PLP (pyridoxal 5′-phosphate), TEoA (triethanolamine buffer), HEPES (HEPES zwitterionic buffer; 4-(2-hydroxyethyl)-piperazineethanesulfonic acid); SFP (shake flask powder); CDS (coding sequence); DNA (deoxyribonucleic acid); RNA (ribonucleic acid); E. coli W3110 (commonly used laboratory E. coli strain, available from the Coli Genetic Stock Center [CGSC], New Haven, CT); HTP (high throughput); HPLC (high pressure liquid chromatography); FIOPC (fold improvements over positive control); Microfluidics (Microfluidics, Corp., Westwood, MA); Sigma-Aldrich (Sigma-Aldrich, St. Louis, MO; Difco (Difco Laboratories, BD Diagnostic Systems, Detroit, MI); Agilent (Agilent Technologies, Inc., Santa Clara, CA); Corning (Corning, Inc., Palo Alto, CA); Dow Corning (Dow Corning, Corp., Midland, MI); and Gene Oracle (Gene Oracle, Inc., Mountain View, CA).

### Example 1

Production of Engineered Polypeptides in pCK110900

The polynucleotide (SEQ ID NO: 1) encoding the polypeptide having threonine aldolase activity (SEQ ID NO: 2), was cloned into the pCK110900 vector system (See e.g., U.S. Pat. No. 9,714,437, which is hereby incorporated by reference in its entirety) and subsequently expressed in E. coli W3110fhuA under the control of the lac promoter. This polynucleotide, and associated polypeptide, was derived from and is a mutant of a threonine aldolase found in Escherichia coli.

The polynucleotide (SEQ ID NO: 157) encoding the polypeptide having threonine aldolase activity (SEQ ID NO: 158), was cloned into the pCK110900 vector system (See e.g., U.S. Pat. No. 9,714,437, which is hereby incorporated by reference in its entirety) and subsequently expressed in E. coli W3110fhuA under the control of the lac promoter. This polynucleotide, and associated polypeptide, was derived from and is a mutant of a threonine aldolase found in Sinorhizobium arboris.

The polynucleotide (SEQ ID NO: 281) encoding the polypeptide having amino acid decarboxylase activity (SEQ ID NO: 282) was cloned into the pCK110900 vector system (See e.g., U.S. Pat. No. 9,714,437, which is hereby incorporated by reference in its entirety) and subsequently expressed in E. coli W3110fhuA under the control of the lac promoter. This polynucleotide, and associated polypeptide, was derived from an amino acid decarboxylase found in Planctomycetaceae bacterium.

In a 96-well format, single colonies were picked and grown in 190 μL LB media containing 1% glucose and 30 μg/mL CAM, at 30° C., 200 rpm, and 85% humidity. Following overnight growth, 20 μL of the grown cultures were transferred into a deep well plate containing 380 μL of TB media with 30 μg/mL CAM. The cultures were grown at 30° C., 250 rpm, with 85% humidity for approximately 2.5 hours. When the optical density (OD₆₀₀) of the cultures reached 0.4-0.6, expression of the threonine aldolase or amino acid decarboxylase gene was induced by the addition of IPTG to a final concentration of 1 mM. Following induction, growth continued for 18-20 hours at 30° C., 250 rpm with 85% humidity. Cells were harvested by centrifugation at 4,000 rpm and 4° C. for 10 minutes; the supernatant was then discarded. The cell pellets were stored at −80° C. until ready for use.

Prior to performing the assay, the cell pellets were thawed and resuspended in 300 μL of lysis buffer containing 1 g/L PLP, 1 g/L lysozyme, 0.5 g/L PMBS and 0.025 μL/mL of commercial DNAse (New England BioLabs, M0303L) in 0.1 M triethanolamine-HCl buffer at pH 7.5. The plates were agitated with medium-speed shaking for 2 hours on a microtiter plate shaker at room temperature. The plates were then centrifuged at 4,000 rpm for 15 minutes at 4° C., and the clarified supernatants were used in the HTP assay reaction described in the following examples.

Shake-flask procedures can be used to generate engineered threonine aldolase or amino acid decarboxylase polypeptide shake-flask powders (SFP), which are useful for secondary screening assays and/or use in the biocatalytic processes described herein. Shake flask powder preparation of enzymes provides a more purified preparation (e.g., up to 30% of total protein) of the engineered enzyme, as compared to the cell lysate used in HTP assays and also allows for the use of more concentrated enzyme solutions. To start the culture, a single colony of E. coli containing a plasmid encoding an engineered polypeptide of interest was inoculated into 5 mL of LB cell culture media with 30 μg/mL CAM and 1% glucose. The culture was grown overnight (at least 16 hours) in an incubator at 37° C., with shaking at 250 rpm. The grown culture was then added to 250 mL of TB media with 30 μg/mL CAM, in a 1 L shake-flask. The 250 mL culture was grown at 30° C. and 250 rpm, for 3.5 hours until OD₆₀₀ reached 0.6-0.8. Expression of the threonine aldolase or amino acid decarboxylase gene was induced by the addition of IPTG to a final concentration of 1 mM, and growth was continued for an additional 18-20 hours. Cells were harvested by transferring the culture into a pre-weighed centrifuge bottle, which was then centrifuged at 4,000 rpm for 20 minutes at 4° C. The supernatant was discarded, and the remaining cell pellet was weighed. In some embodiments, the cell pellet was stored at −80° C. until ready to use. For lysis, the cell pellet was resuspended in 6 mL/g wet cell weight of 25 mM triethanolamine-HCl buffer at pH 7.5 containing 1 g/L PLP and lysed using a 110L MICROFLUIDIZER® processor system (Microfluidics). Cell debris was removed by centrifugation at 10,000 rpm for 60 minutes at 4° C. The clarified lysate was collected, frozen at −80° C., and then lyophilized, using standard methods known in the art. Lyophilization of frozen clarified lysate provides a dry shake-flask powder comprising crude engineered polypeptide.

### Example 2

Evolution and Screening of Engineered Polypeptides Derived from SEQ ID NO: 2 for Improved Production of Compound (3)

The engineered polynucleotide (SEQ ID NO: 1) encoding the polypeptide with threonine aldolase activity of SEQ ID NO: 2 was used to generate the engineered polypeptides of Table 2-1. These polypeptides displayed improved threonine aldolase activity under the desired conditions e.g., the improvement in the formation of the β-hydroxy-α-amino acid, compound (3), from the substrates trifluoroacetone and glycine, compounds (1) and (2), respectively, as compared to the starting polypeptide. The engineered polypeptides, having the amino acid sequences of even-numbered sequence identifiers were generated from the “backbone” amino acid sequence of SEQ ID NO: 2, as described below.

Directed evolution began with the polynucleotide set forth in SEQ ID NO: 1. Libraries of engineered polypeptides were generated using various well-known techniques (e.g., saturation mutagenesis, recombination of previously identified beneficial amino acid differences) and screened using HTP assay and analysis methods that measured the polypeptides' ability to produce compound (3).

The enzyme assays were carried out in 96-well deep-well (1.1 mL total volume) plates, in 100 μL total reaction volume per well. The reactions contained 75 v/v % of undiluted threonine aldolase lysate, prepared as described in EXAMPLE 1, 50 mM trifluoroacetone, compound (1), and 100 mM glycine, compound (2), dissolved in 100 mM triethanolamine-HCl buffer at pH 7.5. The reaction plates were heat-sealed and shaken at 600 rpm at 30° C. for 22 hours.

After overnight incubation (˜22 hours), 300 μL/well of acetonitrile was added to the reaction plates and mixed well. The plates were sealed and centrifuged at 4,000 rpm for 10 min. An aliquot of the supernatant was removed and further diluted 10-fold into a 1:1 solution of water in acetonitrile. After dilution, the reactions were analyzed for formation of compound (3) on the Agilent RapidFire 365 high throughput mass spectrometer, using the manufacturer's protocols.

TABLE 2-1
ThrAldo Activity for the Production of Compound (3) Relative to SEQ ID NO: 2

| SEQ ID NO: | Amino Acid Differences | Product Area Fold Improvement |
|---|---|---|
| (nt/aa) | (Relative to SEQ ID NO: 2) | (Relative to SEQ ID NO: 2)¹ |
| 3/4 | T10A/G198T | +++ |
| 5/6 | L249T | +++ |
| 7/8 | L55Y/Q60S/A173T | +++ |
| 9/10 | Q60S/Q250G/H253W | +++ |
| 11/12 | I170S | +++ |
| 13/14 | D7E/T8L/I170V/F171A | +++ |
| 15/16 | T8V | +++ |
| 17/18 | F171A/N172G/I193T/L195T/L249M | +++ |
| 19/20 | V174L/R211C/H253W | +++ |
| 21/22 | C194T/S196H | +++ |
| 23/24 | I170L/H253W | +++ |
| 25/26 | F171A/L195T | +++ |
| 27/28 | Q60S/H253W | +++ |
| 29/30 | G138C/R169H | +++ |
| 31/32 | S205E | +++ |
| 33/34 | I170V/F171A | ++ |
| 35/36 | L55Y/Q60S | ++ |
| 37/38 | F171G/T201A/H253R | ++ |
| 39/40 | I170V/L249T | ++ |
| 41/42 | I193T/L195T | ++ |
| 43/44 | L195T/S196H | ++ |
| 45/46 | N245E | ++ |
| 47/48 | F171A/L249T | ++ |
| 49/50 | L55Y/Q60S/Q250G | ++ |
| 51/52 | G138A/S205E | ++ |
| 53/54 | P102H | ++ |
| 55/56 | Q60S/A247L/H253W | ++ |
| 57/58 | H253W | ++ |
| 59/60 | T10A/R169H/S205E | ++ |
| 61/62 | V174L/H253W | ++ |
| 63/64 | L55S/I170L/H253W | ++ |
| 65/66 | L55Y/Q60S/A247L/Q250G | ++ |
| 67/68 | H253R | ++ |
| 69/70 | G138C/G167M | ++ |
| 71/72 | T201P/S205E/V246F | ++ |
| 73/74 | Q250G/H253W | ++ |
| 75/76 | G138C/G167M/H312P | ++ |
| 77/78 | L55T/Q60S/H253R | ++ |
| 79/80 | F171A/H253W | + |
| 81/82 | N140M/V143S/A176L/L206A | + |
| 83/84 | A173T/Q250G/H253W | + |
| 85/86 | I170G/F171A/N172G/I193T/C194T | + |
| 87/88 | A173T/A247L/Q250G/A324T | + |
| 89/90 | L55Y/Q60S/Q250G/H253W | + |
| 91/92 | R169H | + |
| 93/94 | R11L/L206A | + |
| 95/96 | N140F/V143S/L206A | + |
| 97/98 | I170V/S196H | + |
| 99/100 | L55Y/Q60S/F171G/V174L/H253W | + |
| 101/102 | N172G | + |
| 103/104 | G138A/G167M | + |
| 105/106 | F171A/N172G | + |
| 107/108 | I193T/C194T | + |
| 109/110 | R11L/A81L/L206A | + |
| 111/112 | N276H | + |
| 113/114 | F171A/V174L/T201C/H253R | + |
| 115/116 | F171A | + |
| 117/118 | F191A/H253W | + |
| 119/120 | G138A/R169H/S205E | + |
| 121/122 | L206A | + |
| 123/124 | T57S/N140F/V143S/L206A | + |
| 125/126 | T10A | + |
| 127/128 | I170V/I193T/L195T/S196H | + |
| 129/130 | G138C/R169H/N276H | + |
| 131/132 | D7E/I170V/F171A/N172G/L195T/S196H | + |
| 133/134 | T10A/G138C/S205E/H312P | + |
| 135/136 | L195T | + |
| 137/138 | G138C/N245E | + |
| 139/140 | T8L/I170S/C194T/S196H | + |
| 141/142 | G167M/S205E | + |
| 143/144 | I193T | + |
| 145/146 | I170S/N172G | + |
| 147/148 | S196H | + |
| 149/150 | D7E/I170S/F171A/1193T | + |
| 151/152 | G198T/T201P/S205E | + |
| 153/154 | T10A/G167M | + |
| 155/156 | C194T | + |

Levels of increased activity were determined relative to the reference polypeptide of SEQ ID NO: 2 and defined as follows: “+” 1.61 to 2.15, “+” > 2.15, “+” > 2.36.

### Example 3

Evaluation of Engineered Polypeptides Derived from SEQ ID NO: 2 for Improved Production of Compound (4)

Hit variants from EXAMPLE 2 were grown in 250 mL shake flasks as described in EXAMPLE 1 to generate lyophilized enzyme powders. The activity of each of the enzyme powders was evaluated in a coupled reaction with the amino acid decarboxylase described in SEQ ID NO: 282 for the production of compound (4) starting with compounds (1) and (2). This reaction contained 75 g/L of the lyophilized threonine aldolase enzyme variant, 25 g/L of lyophilized AADC described by SEQ ID NO: 282, 50 mM trifluoroacetone, compound (1), 100 mM glycine, compound (2), and 0.75 g/L PLP dissolved in 0.1 M triethanolamine-HCl buffer at pH 7.5 with a total reaction volume of 100 μL. The reaction was allowed to react at 30° C., with shaking, for 22 hours. The reaction was then quenched by extracting the product into 3 volumes (300 μL) of ethyl acetate. This was analyzed by gas chromatography (GC) using the method described in Table 3-1. The methods provided herein find use in analyzing the variants produced using the present invention. However, it is not intended that present invention be limited to the methods described herein, as there are other suitable methods known in the art that are applicable to the analysis of the variants provided herein and/or produced using the methods provided herein.

TABLE 3-1
GC Parameters
Method for Determining Conversion of
Compound (1) and (2) to Compound (4)

| Instrument | Agilent 6890 GC | |
|---|---|---|
| Column | Restek Rtx-5 Amine, 30 m × 320 μm × | |
| | 1 μm film thickness (p/n 12354) | |
| Carrier gas and flow rate | Helium, 6.0 mL/min (constant flow control) | |
| Column temperature | 60° C. to 100° C. at 20° C./min, | |
| | hold at 100° C. for 2 min | |
| Detector and temperature | FID, 250° C. | |
| Inlet temperature and split | 220° C., 50:1 | |
| ratio | | |
| Injection volume | 2 | μL |
| Product retention time | 1.65 | min |

### Example 4

Evolution and Screening of Engineered Polypeptides Derived from SEQ ID NO: 158 for Improved Production of Compound (3)

The engineered polynucleotide (SEQ ID NO: 157) encoding the polypeptide with threonine aldolase activity of SEQ ID NO: 158 was used to generate the engineered polypeptides of Table 4-1. These polypeptides displayed improved threonine aldolase activity under the desired conditions e.g., the improvement in the formation of the β-hydroxy-α-amino acid, compound (3), from the substrates trifluoroacetone and glycine, compounds (1) and (2), respectively, as compared to the starting polypeptide. The engineered polypeptides, having the amino acid sequences of even-numbered sequence identifiers were generated from the “backbone” amino acid sequence of SEQ ID NO: 158 as described in EXAMPLE 2.

TABLE 4-1
ThrAldo Activity for the Production of Compound (3) Relative to SEQ ID NO: 158

| | | Product Area Fold |
|---|---|---|
| SEQ ID | | Improvement |
| NO: | Amino Acid Differences | (Relative to SEQ |
| (nt/aa) | (Relative to SEQ ID NO: 158) | ID NO: 158)¹ |
| 159/160 | W254M/L255K/L257Y/A258Q | +++ |
| 161/162 | Q141S/W254M/L255K/L257Y/A258Q | +++ |
| 163/164 | S199T/F200K/K204E | +++ |
| 165/166 | A210R/W254M | +++ |
| 167/168 | G172M/R174H/F245K | +++ |
| 169/170 | S199T/F200K/T203S/G206L/Y248A | +++ |
| 171/172 | A258Q | +++ |
| 173/174 | C207M/E211S/L257Y/A258Q | +++ |
| 175/176 | C207M/A210P/E211Q/L255K/L257Y | +++ |
| 177/178 | F175I/F245K | +++ |
| 179/180 | Q141S/L255K/L257Y/A258Q | +++ |
| 181/182 | C207M/A210R/A212G/L257Y | +++ |
| 183/184 | C207L/A212G/L255K/A258Q | ++ |
| 185/186 | Q141S/A258S | ++ |
| 187/188 | Q141S/W254M/L257Y/A258Q | ++ |
| 189/190 | Q141S/W254M/A258Q | ++ |
| 191/192 | G172M/R174H/F175V/F200K/T203S/N205E/F245K | ++ |
| 193/194 | G172M/R174H/F175V/F245K | ++ |
| 195/196 | S199T/F200K/T203S/K204E/G206L/F245K | ++ |
| 197/198 | C207M/E211G | ++ |
| 199/200 | W254M/L257Y | ++ |
| 201/202 | R174H/F175I/F245K/Y248A | ++ |
| 203/204 | S199T/F200K/G206L | ++ |
| 205/206 | G172M/F175V | ++ |
| 207/208 | E211A/W254M/A258S | ++ |
| 209/210 | R174H/F175I/S199T/F200K/T203S/G206L/F245K/Y248A | ++ |
| 211/212 | Q141S/L257Y/A258Q | ++ |
| 213/214 | R174H/F175I/F245K | ++ |
| 215/216 | F245K/Y248A | ++ |
| 217/218 | C207M/E211G/A212G/W254M/L255K/L257Y/A258Q | ++ |
| 219/220 | E211S/W254M/L257Y/A258Q | + |
| 221/222 | S199T/T203S/K204E | + |
| 223/224 | R174H/F175I/S199T/F200K/N205E | + |
| 225/226 | Q141S/A212G/W254M/L257Y/A258S | + |
| 227/228 | G172M/R174H/F175V | + |
| 229/230 | S199T/T203S/K204E/G206L/F245K/Y248A | + |
| 231/232 | Q141S/A212G/L257Y/A258Q | + |
| 233/234 | T203S/F245K/Y248A | + |
| 235/236 | W254M/L255K/A258Q | + |
| 237/238 | R174H/F245K/Y248A | + |
| 239/240 | A210R/A212G/A258Q | + |
| 241/242 | A178G/C207L/E211S/W254M/L255K/L257Y/A258S | + |
| 243/244 | G172M/R174H/S199T/F200K/T203S/N205E/Y248A | + |
| 245/246 | G172M/S199T/T203S/K204E/G206L | + |
| 247/248 | Q141S/W254M/L255K/A258Q | + |
| 249/250 | A178G/S181C/E211S/W254M/L255K/A258Q | + |
| 251/252 | R174H/F175V/S199T/T203S/K204E/N205E | + |
| 253/254 | C207M/E211S | + |
| 255/256 | F175S/A176Q/S199T | + |
| 257/258 | Q141S/C207M/A210R/W254M/L257Y/A258Q | + |
| 259/260 | E211S/L257Y/A258Q | + |
| 261/262 | Q141S/C207L/A210R/W254M/A258Q | + |
| 263/264 | G172M/R174H/F175I/F245K/Y248A | + |
| 265/266 | C207M/A210R/L255K | + |
| 267/268 | Q141S/E211S/L257Y/A258Q | + |
| 269/270 | Q141S/C207L/L255K/A258S | + |
| 271/272 | G172M/R174H/Y248A | + |
| 273/274 | Q141S/E211G/A212G/W254M | + |
| 275/276 | R174H/F175V | + |
| 277/278 | N128V | + |
| 279/280 | I15L/L268Q/L317W | + |

Levels of increased activity were determined relative to the reference polypeptide of SEQ ID NO: 158 and defined as follows: “+” 1.95 to 2.69, “+” > 2.69, “+” > 3.03.

### Example 5

Evaluation of Engineered Polypeptides Derived from SEQ ID NO: 158 for Improved Production of Compound (4)

Hit variants from EXAMPLE 4 were evaluated in a coupled reaction with the amino acid decarboxylase described in SEQ ID NO: 282 for the formation of compound (4). The method used is described in EXAMPLE 3.

### Example 6

Evolution and Screening of Engineered Polypeptides Derived from SEQ ID NO: 282 for Improved Production of Compound (4)

The engineered polynucleotide (SEQ ID NO: 281) encoding the polypeptide with amino acid decarboxylase activity of SEQ ID NO: 282 was used to generate the engineered polypeptides of Table 6-1. These polypeptides displayed improved amino acid decarboxylase activity under the desired conditions e.g., the improvement in the formation of the amino alcohol, compound (4), from the β-hydroxy-α-amino acid, compound (3), that was produced in situ from the substrates trifluoroacetone and glycine, compounds (1) and (2), respectively, catalyzed by a threonine aldolase mutant (SEQ ID NO: 200), as compared to the starting polypeptide. Some polypeptides displayed improved stereoselectivity of the produced amino alcohol product (4), with some giving an increase in the enantioselectivity towards the (S)-amino alcohol product compared to the starting polypeptide and are noted in Table 6-1. The engineered polypeptides, having the amino acid sequences of even-numbered sequence identifiers were generated from the “backbone” amino acid sequence of SEQ ID NO: 282, as described below together with the analytical method described in Table 6-2.

Directed evolution began with the polynucleotide set forth in SEQ ID NO: 281. Libraries of engineered polypeptides were generated using various well-known techniques (e.g., saturation mutagenesis, recombination of previously identified beneficial amino acid differences) and screened using HTP assay and analysis methods, described below, that measured the polypeptides' ability to produce compound (4).

The enzyme assays were carried out in 96-well deep-well (1.1 mL total volume) plates, in 100 μL total reaction volume per well. The reactions contained 75 v/v % of undiluted amino acid decarboxylase lysate, prepared as described in EXAMPLE 1 except the lysis volume was 200 μL instead of 300 μL, 100 mM trifluoroacetone, compound (1), 100 mM glycine, compound (2), 10 g/L threonine aldolase (SEQ ID NO: 282), 0.75 g/L PLP in 0.1 M triethanolamine-HCl buffer at pH 7.0. The reaction plates were heat-sealed and shaken at 600 rpm and 30° C.

After overnight incubation (˜22 hours), 300 μL/well of ethyl acetate containing 1 v/v % triethylamine was added to the reaction plates. The plates were sealed, mixed well, and centrifuged at 4,000 rpm for 10 min. A 200 μL aliquot of the top organic phase was removed and added to a shallow well 96-well plate. These samples were then analyzed by chiral GC to determine the activity and stereoselectivity of the enzyme variants using the analytical method described in Table 6-2.

TABLE 6-1
AADC Activity for the Production of Compound (4) Relative to SEQ ID NO: 282

| SEQ ID | | Product Area Fold |
|---|---|---|
| NO: | Amino Acid Differences | Improvement (Relative |
| (nt/aa) | (Relative to SEQ ID NO: 282) | to SEQ ID NO: 282)¹ |
| 283/284 | G248S | +++, # |
| 285/286 | N279P | +++ |
| 287/288 | I245W | +++ |
| 289/290 | S66N/M86F/A198G/I235V/A329S | +++ |
| 291/292 | M86F/M162I/N186D/V187I/S202Y/H203Y/I235V/A329S | +++ |
| 293/294 | N56D | +++, # |
| 295/296 | I74F/A121L/F194L/L233I/R253K | +++ |
| 297/298 | I74F/F110Y/L233I/R324W | +++ |
| 299/300 | I74F/A121L/F194L/L233I/R324W | ++ |
| 301/302 | I74F/F194L/L233I | ++ |
| 303/304 | A174C | ++ |
| 305/306 | G280A | ++ |
| 307/308 | I74F/F110Y/R324W | ++ |
| 309/310 | I74F/F110Y/A121L/F194L | ++ |
| 311/312 | I74F/F194L/R253K | ++ |
| 313/314 | E258S | ++ |
| 315/316 | V55I/I74F/F110Y/F194L/L233I/I281H/R324W | ++ |
| 317/318 | V55I/I74F/F110Y/A121L/F194L/L233I/R253K | ++ |
| 319/320 | I74F/F194L | ++ |
| 321/322 | P49N | ++ |
| 323/324 | S66N/V290I/A329S | ++ |
| 325/326 | V55I/174F/F110Y/A211S | + |
| 327/328 | S66N/S202Y/V290I/A329S | + |
| 329/330 | A80K | + |
| 331/332 | A63T/P398L | + |
| 333/334 | S66N/A198G/S202Y/V290I/F316L/A329S | + |
| 335/336 | I245E | + |
| 337/338 | E103G | + |
| 339/340 | N350E | + |
| 341/342 | A198S | + |
| 343/344 | M86T | + |
| 345/346 | M162I/V187I/S202Y/I235V/T252A/V290I/F316L | + |
| 347/348 | G183V | + |
| 349/350 | L135I | + |
| 351/352 | V55I/F194L/A211S/L233I/R253K | + |
| 353/354 | V55I/I74F/A121L/L233I/R253K/R324W | + |
| 355/356 | V55I/174F/F194L/L233I | + |
| 357/358 | V551/A211S/R253K/R324W | + |
| 359/360 | R72E | + |
| 361/362 | L48V | + |
| 363/364 | M162L | + |
| 365/366 | R72A | + |

Levels of increased activity were determined relative to the reference polypeptide of SEQ ID NO: 282 and defined as follows: “+” 1.51 to 1.77, “+” > 1.77, “+” > 1.98.
Variants with a “#” showed improved selectivity towards the (S)-enantiomer relative to the reference polypeptide of SEQ ID NO: 282.

TABLE 6-2
GC Parameters
Method for Determining Conversion and Stereoselectivity
of Compound (1) and (2) to Compound (4)

| | |
|---|---|
| Instrument | Agilent 6890 GC |
| Column | Supelco β-DEX225, 30 m × 250 μm × |
| | 0.25 μm film thickness (p/n 24348) |
| Carrier gas and flow rate | Helium, 4.0 mL/min (constant flow control) |
| Column temperature | 110° C. to 150° C. at 20° C./min, |
| | hold at 150° C. for 0.2 min |
| Detector and temperature | FID, 250° C. |
| Inlet temperature and split | 220° C., 25:1 |
| ratio | |
| Injection volume | 2 μL |
| Product retention time | (S)-amino alcohol: 1.85 min |
| | (R)-amino alcohol: 1.95 min |

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

1. An engineered threonine aldolase polypeptide comprising a polypeptide sequence having at least 80%, 85%, 86%, 87%, 88%, 89%, 90%, 91%, 92%, 93%, 94%, 95%, 96%, 97%, 98%, 99%, or more sequence identity to SEQ ID NO: 2, or a functional fragment thereof, wherein said engineered threonine aldolase polypeptide comprises at least one substitution or substitution set in said polypeptide sequence, and wherein the amino acid positions of said polypeptide sequence are numbered with reference to SEQ ID NO: 2.

2. (canceled)

3. The engineered threonine aldolase polypeptide of claim 1, wherein said polypeptide sequence has at least 80%, 85%, 86%, 87%, 88%, 89%, 90%, 91%, 92%, 93%, 94%, 95%, 96%, 97%, 98%, 99%, or more sequence identity to SEQ ID NO: 2, wherein said engineered threonine aldolase polypeptide comprises at least one substitution or substitution set in said polypeptide sequence at one or more positions selected from 10/198, 7/8/170/171, 7/170/171/172/195/196, 7/170/171/193, 8, 8/170/194/196, 10, 10/138/205/312, 10/167, 10/169/205, 10/198, 11/81/206, 11/206, 55/60, 55/60/171/174/253, 55/60/173, 55/60/247/250, 55/60/250, 55/60/250/253, 55/60/253, 55/170/253, 57/140/143/206, 60/247/253, 60/250/253, 60/253, 102, 138/167, 138/167/312, 138/169, 138/169/205, 138/169/276, 138/205, 138/245, 140/143/176/206, 140/143/206, 167/205, 169, 170, 170/171, 170/171/172/193/194, 170/172, 170/193/195/196, 170/196, 170/249, 170/253, 171, 171/172, 171/172/193/195/249, 171/174/201/253, 171/195, 171/201/253, 171/249, 171/253, 172, 173/247/250/324, 173/250/253, 174/211/253, 174/253, 191/253, 193, 193/194, 193/195, 194, 194/196, 195, 195/196, 196, 198/201/205, 201/205/246, 205, 206, 245, 249, 250/253, 253, and 276, and wherein the amino acid positions of said polypeptide sequence are numbered with reference to SEQ ID NO: 2.

4. An engineered threonine aldolase polypeptide comprising a polypeptide sequence having at least 80%, 85%, 86%, 87%, 88%, 89%, 90%, 91%, 92%, 93%, 94%, 95%, 96%, 97%, 98%, 99%, or more sequence identity to SEQ ID NO: 158, or a functional fragment thereof, wherein said engineered threonine aldolase polypeptide comprises at least one substitution or substitution set in said polypeptide sequence, and wherein the amino acid positions of said polypeptide sequence are numbered with reference to SEQ ID NO: 158.

5. (canceled)

6. The engineered threonine aldolase polypeptide of claim 4, wherein said polypeptide sequence has at least 80%, 85%, 86%, 87%, 88%, 89%, 90%, 91%, 92%, 93%, 94%, 95%, 96%, 97%, 98%, 99%, or more sequence identity to SEQ ID NO: 158, wherein said engineered threonine aldolase polypeptide comprises at least one substitution or substitution set in said polypeptide sequence at one or more positions selected from 141/254/255/257/258, 15/268/317, 128, 141/207/210/254/257/258, 141/207/210/254/258, 141/207/255/258, 141/211/212/254, 141/211/257/258, 141/212/254/257/258, 141/212/257/258, 141/254/255/258, 141/254/257/258, 141/254/258, 141/255/257/258, 141/257/258, 141/258, 172/174/175, 172/174/175/200/203/205/245, 172/174/175/245, 172/174/175/245/248, 172/174/199/200/203/205/248, 172/174/245, 172/174/248, 172/175, 172/199/203/204/206, 174/175, 174/175/199/200/203/206/245/248, 174/175/199/200/205, 174/175/199/203/204/205, 174/175/245, 174/175/245/248, 174/245/248, 175/176/199, 175/245, 178/181/211/254/255/258, 178/207/211/254/255/257/258, 199/200/203/204/206/245, 199/200/203/206/248, 199/200/204, 199/200/206, 199/203/204, 199/203/204/206/245/248, 203/245/248, 207/210/211/255/257, 207/210/212/257, 207/210/255, 207/211, 207/211/212/254/255/257/258, 207/211/257/258, 207/212/255/258, 210/212/258, 210/254, 211/254/257/258, 211/254/258, 211/257/258, 245/248, 254/255/257/258, 254/255/258, 254/257, and 258, and wherein the amino acid positions of said polypeptide sequence are numbered with reference to SEQ ID NO: 158.

7. An engineered amino acid decarboxylase polypeptide comprising a polypeptide sequence having at least 80%, 85%, 86%, 87%, 88%, 89%, 90%, 91%, 92%, 93%, 94%, 95%, 96%, 97%, 98%, 99%, or more sequence identity to SEQ ID NO: 282, or a functional fragment thereof, wherein said engineered amino acid decarboxylase polypeptide comprises at least one substitution or substitution set in said polypeptide sequence, and wherein the amino acid positions of said polypeptide sequence are numbered with reference to SEQ ID NO: 282.

8. (canceled)

9. The engineered amino acid decarboxylase polypeptide of claim 7, wherein said polypeptide sequence has at least 80%, 85%, 86%, 87%, 88%, 89%, 90%, 91%, 92%, 93%, 94%, 95%, 96%, 97%, 98%, 99%, or more sequence identity to SEQ ID NO: 282, wherein said engineered amino acid decarboxylase polypeptide comprises at least one substitution or substitution set in said polypeptide sequence at one or more positions selected from 248, 48, 49, 55/74/110/121/194/233/253, 55/74/110/194/233/281/324, 55/74/110/211, 55/74/121/233/253/324, 55/74/194/233, 55/194/211/233/253, 55/211/253/324, 56, 63/398, 66/86/198/235/329, 66/198/202/290/316/329, 66/202/290/329, 66/290/329, 72, 74/110/121/194, 74/110/233/324, 74/110/324, 74/121/194/233/253, 74/121/194/233/324, 74/194, 74/194/233, 74/194/253, 80, 86, 86/162/186/187/202/203/235/329, 103, 135, 162, 162/187/202/235/252/290/316, 174, 183, 198, 245, 258, 279, 280, and 350, and 258, and wherein the amino acid positions of said polypeptide sequence are numbered with reference to SEQ ID NO: 282.

10. The engineered threonine aldolase polypeptide of claim 1, wherein said engineered threonine aldolase polypeptide comprises an amino acid sequence with at least 80% sequence identity to any even-numbered sequence set forth in SEQ ID NO: 4 to SEQ ID NO: 156.

11. The engineered threonine aldolase polypeptide of claim 4, wherein said engineered threonine aldolase polypeptide comprises an amino acid sequence with at least 80% sequence identity to any even-numbered sequence set forth in SEQ ID NO: 160 to SEQ ID NO: 280.

12. The engineered amino acid decarboxylase polypeptide of claim 7, wherein said engineered amino acid decarboxylase polypeptide comprises an amino acid sequence with at least 80% sequence identity to any even-numbered sequence set forth in SEQ ID NO: 284 to SEQ ID NO: 366.

13. The engineered threonine aldolase polypeptide of claim 1, wherein said engineered threonine aldolase polypeptide comprises a polypeptide sequence set forth in any of the even numbered sequences of SEQ ID NOs: 4-156.

14. The engineered threonine aldolase polypeptide of claim 4, wherein said engineered threonine aldolase polypeptide comprises a polypeptide sequence set forth in any of the even numbered sequences of SEQ ID NOs: 160-280.

15. The engineered amino acid decarboxylase polypeptide of claim 7, wherein said engineered amino acid decarboxylase polypeptide comprises a polypeptide sequence set forth in any of the even numbered sequences of SEQ ID NOs: 284-366.

16. The engineered threonine aldolase polypeptide of claim 1, wherein said engineered threonine aldolase polypeptide comprises a polypeptide sequence that comprises improved production of compound (3) 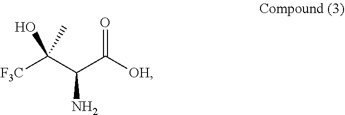
as compared to the engineered threonine aldolase polypeptide of SEQ ID NO: 2.

17. The engineered threonine aldolase polypeptide of claim 4, wherein said engineered threonine aldolase polypeptide comprises a polypeptide sequence that comprises improved production of compound (3) 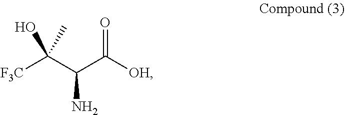
as compared to the engineered threonine aldolase polypeptide of SEQ ID NO: 158.

18. The engineered amino acid decarboxylase polypeptide of claim 7, wherein said engineered amino acid decarboxylase polypeptide comprises a polypeptide sequence that comprises improved production of compound (4) 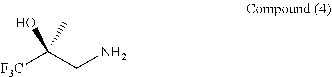
and/or comprises improved enantioselectivity, as compared to the engineered amino acid decarboxylase polypeptide of SEQ ID NO: 282.

19-21. (canceled)

22. The engineered polypeptide of claim 1, wherein said engineered polypeptide is purified.

23. A composition comprising at least one engineered polypeptide provided in claim 1.

24. An engineered polynucleotide encoding at least one engineered polypeptide of claim 1.

25.-28. (canceled)

29. A vector comprising the engineered polynucleotide of claim 24.

30. (canceled)

31. A host cell comprising the vector of claim 29

32. (canceled)

33. A method of producing an engineered polypeptide in a host cell, comprising culturing the host cell of claim 31, in a culture medium under suitable conditions, such that at least one engineered polypeptide is produced.

34.-35. (canceled)


SEQUENCE LISTING (366 sequences)

SEQ ID NO: 1 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 2 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 3 - DNA, 999 bp, synthetic construct
  misc_feature 1..999: note = Variant of Threonine Aldolase from Escherichia coli
  source 1..999: mol_type = other DNA
atgattgatttacgcagtgataccgttgctcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacggtcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaaccctggggactccagtcggttcgttactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 4 - protein, 333 aa, synthetic construct
  REGION 1..333: note = Variant of Threonine Aldolase from Escherichia coli
MIDLRSDTVARPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKTLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 5 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatttttaacgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcacgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 6 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARTQEDHDNAAWMAEQLREAGADVMRQDTNMLFVRVGEENAAALGEYMKARNVLINASPIVRLVTHLDVSREQLAEVAAHWRAFLAR

SEQ ID NO: 7 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccattttttaccctaccggcactagcgccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatactgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 8 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFYPTGTSANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNTVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 9 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactagcgccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgggtgaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 10 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTSANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLGEDWDNAAWMAEQLREAGADVMRQDTNMLFVRVGEENAAALGEYMKARNVLINASPIVRLVTHLDVSREQLAEVAAHWRAFLAR

SEQ ID NO: 11 - DNA, 999 bp, synthetic construct
  misc_feature 1..999: note = Variant of Threonine Aldolase from Escherichia coli
  source 1..999: mol_type = other DNA
atgattgatttacgcagtgatacggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcagttttaacgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcctgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 12 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARSFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 13 - DNA, 999 bp, synthetic construct
  misc_feature 1..999: note = Variant of Threonine Aldolase from Escherichia coli
  source 1..999: mol_type = other DNA
atgattgatttacgcagtgagctggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcgtagctaacgccgtggtggcttacggctgcgaactgaaagagatcacgcaatattgtgattcgttcaccatttgtctgagtaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 14 - protein, 333 aa, synthetic construct
  REGION 1..333: note = Variant of Threonine Aldolase from Escherichia coli
MIDLRSELVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARVANAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 15 - DNA, 999 bp, synthetic construct
  misc_feature 1..999: note = Variant of Threonine Aldolase from Escherichia coli
  source 1..999: mol_type = other DNA
atgattgatttacgcagtgatgtggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatttttaacgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 16 - protein, 333 aa, synthetic construct
  REGION 1..333: note = Variant of Threonine Aldolase from Escherichia coli
MIDLRSDVVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 17 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcattgctggcgccgtggtggcttacggctgcgaactgaaagagatcacgcaatattgtgattcgttcaccacttgtactagtaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcatgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 18 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIAGAVVAYGCELKEITQYCDSFTTCTSKGLGTPVGSLLVGNRDYIKRAIRWRKMTGGGMRQSGILAAAGIYALKNNVARMQEDHDNAAWMAEQLREAGADVMRQDTNMLFVRVGEENAAALGEYMKARNVLINASPIVRLVTHLDVSREQLAEVAAHWRAFLAR

SEQ ID NO: 19 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaagccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccctggtggcttacggctgcgaactgaaagagatcacgcaatattgtgattcgtttaccatttgcctgtcgaaaggtcttgggacgccagtcggttcattactggtcggtaattgtgattacattaaacgtgccattcgctggcggaaaatgacaggtggcgggatgcgccagtccggcattctggcagccgccgggatatatgccctgaaaaataacgttgcgcgcttgcaggaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 20 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNALVAYGCELKEITQYCDSFTICLSKGLGTPVGSLLVGNCDYIKRAIRWRKMTGGGMRQSGILAAAGIYALKNNVARLQEDWDNAAWMAEQLREAGADVMRQDTNMLFVRVGEENAAALGEYMKARNVLINASPIVRLVTHLDVSREQLAEVAAHWRAFLAR

SEQ ID NO: 21 - DNA, 999 bp, synthetic construct
  misc_feature 1..999: note = Variant of Threonine Aldolase from Escherichia coli
  source 1..999: mol_type = other DNA
atgattgatttacgcagtgatacggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccattacgctgcataaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 22 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTITLHKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 23 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaagccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcctgtttaatgctgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 24 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARLFNAVVAYGCELKEITQYCDSFTICLSKGLGTPVGSLLVGNRDYIKRAIRWRKMTGGGMRQSGILAAAGIYALKNNVARLQEDWDNAAWMAEQLREAGADVMRQDTNMLFVRVGEENAAALGEYMKARNVLINASPIVRLVTHLDVSREQLAEVAAHWRAFLAR

SEQ ID NO: 25 - DNA, 999 bp, synthetic construct
  misc_feature 1..999: note = Variant of Threonine Aldolase from Escherichia coli
  source 1..999: mol_type = other DNA
atgattgatttacgcagtgatacggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcattgctaacgccgtggtggcttacggctgcgaactgaaagagatcacgcaatattgtgattcgttcaccatttgtactagtaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 26 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIANAVVAYGCELKEITQYCDSFTICTSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 27 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactagcgccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 28 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTSANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLQEDWDNAAWMAEQLREAGADVMRQDTNMLFVRVGEENAAALGEYMKARNVLINASPIVRLVTHLDVSREQLAEVAAHWRAFLAR

SEQ ID NO: 29 - DNA, 999 bp, synthetic construct
  misc_feature 1..999: note = Variant of Threonine Aldolase from Escherichia coli
  source 1..999: mol_type = other DNA
atgattgatttacgcagtgataccgttactcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaactgtcacaacggcaaagtgttgccgcgggaatacctgaaagaagcatgggaatttacccgcgagcgcaatctggcgctgcatgttgacggggcgcacatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 30 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENCHNGKVLPREYLKEAWEFTRERNLALHVDGAHIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 31 - DNA, 999 bp, synthetic construct
  misc_feature 1..999: note = Variant of Threonine Aldolase from Escherichia coli
  source 1..999: mol_type = other DNA
atgattgatttacgcagtgataccgttactcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggggcgcgcatctttaatgccgtggtggcttacggctgcgaactgaaagagatcacgcaatattgtgattcgttcaccatttgcctgtcgaaagggctggggactccagtcggtgagttactcgtcggtaatcgtgattacattaaacgtgccattcgctggcggaaaatgacaggtggcgggatgcgccagtccggcattctggctgccgccgggatatatgccctgaaaaataatgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 32 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGELLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 33 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcgtagctaacgccgtggtggcttacggctgcgaactgaaagagatcacgcaatattgtgattcgttcaccatttgtctgagtaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 34 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARVANAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 35 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccattttttaccctaccggcactagcgccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 36 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFYPTGTSANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 37 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaagccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatcggtaatgccgtggtggcttacggctgcgaactgaaagagatcacgcaatattgtgattcgtttaccatttgcctgtcgaaaggtcttggggcgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccgcgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctagcgcgt

SEQ ID NO: 38 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIGNAVVAYGCELKEITQYCDSFTICLSKGLGAPVGSLLVGNRDYIKRAIRWRKMTGGGMRQSGILAAAGIYALKNNVARLQEDRDNAAWMAEQLREAGADVMRQDTNMLFVRVGEENAAALGEYMKARNVLINASPIVRLVTHLDVSREQLAEVAAHWRAFLAR

SEQ ID NO: 39 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcgtatttaacgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcacgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 40 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARVFNAVVAYGCELKEITQYCDSFTICLSKGLGTPVGSLLVGNRDYIKRAIRWRKMTGGGMRQSGILAAAGIYALKNNVARTQEDHDNAAWMAEQLREAGADVMRQDTNMLFVRVGEENAAALGEYMKARNVLINASPIVRLVTHLDVSREQLAEVAAHWRAFLAR

SEQ ID NO: 41 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccacttgtactagtaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 42 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTTCTSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 43 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatttttaacgccgtggtggcttacggctgcgaactgaaagagatcacgcaatattgtgattcgttcaccatttgtactcataaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcctgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 44 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICTHKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 45 - DNA, 999 bp, synthetic construct
  misc_feature 1..999: note = Variant of Threonine Aldolase from Escherichia coli
  source 1..999: mol_type = other DNA
atgattgatttacgcagtgataccgttactcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaagggctggggactccagtcggttcgttactcgtcggtaatcgtgattacattaaacgtgccattcgctggcggaaaatgacaggtggcgggatgcgccagtccggcattctggctgccgccgggatatatgccctgaaaaatgaggttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 46 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNEVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 47 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcattgctaacgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcacgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 48 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIANAVVAYGCELKEITQYCDSFTICLSKGLGTPVGSLLVGNRDYIKRAIRWRKMTGGGMRQSGILAAAGIYALKNNVARTQEDHDNAAWMAEQLREAGADVMRQDTNMLFVRVGEENAAALGEYMKARNVLINASPIVRLVTHLDVSREQLAEVAAHWRAFLAR

SEQ ID NO: 49 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccattttttaccctaccggcactagcgccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgggtgaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 50 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFYPTGTSANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLGEDHDNAAWMAEQLREAGADVMRQDTNMLFVRVGEENAAALGEYMKARNVLINASPIVRLVTHLDVSREQLAEVAAHWRAFLAR

SEQ ID NO: 51 - DNA, 999 bp, synthetic construct
  misc_feature 1..999: note = Variant of Threonine Aldolase from Escherichia coli
  source 1..999: mol_type = other DNA
atgattgatttacgcagtgataccgttactcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgctcacaacggcaaagtgttgccgcgggaatacctgaaagaagcatgggaatttacccgcgagcgcaatctggcgctgcatgttgacggggcgcgcatctttaatgccgtggtggcttacggctgcgaactgaaagagatcacgcaatattgtgattcgttcaccatttgcctgtcgaaaggtcttgggactccagtcggtgagttactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaacatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 52 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENAHNGKVLPREYLKEAWEFTRERNLALHVDGARIFNAVVAYGCELKEITQYCDSFTICLSKGLGTPVGELLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 53 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaagccaacctggtcgctctgctcagtcactgcgaacgcggcgaagagtatattgtcggtcaggccgcgcataactatctgtttgaagccggtggcgcggcggtgctgggcagtattcaaccgcaacacatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 54 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQHIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 55 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactagcgccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttctgcgcttgcaggaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 56 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTSANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVLRLQEDWDNAAWMAEQLREAGADVMRQDTNMLFVRVGEENAAALGEYMKARNVLINASPIVRLVTHLDVSREQLAEVAAHWRAFLAR

SEQ ID NO: 57 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 58 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLQEDWDNAAWMAEQLREAGADVMRQDTNMLFVRVGEENAAALGEYMKARNVLINASPIVRLVTHLDVSREQLAEVAAHWRAFLAR

SEQ ID NO: 59 - DNA, 999 bp, synthetic construct
  misc_feature 1..999: note = Variant of Threonine Aldolase from Escherichia coli
  source 1..999: mol_type = other DNA
atgattgatttacgcagtgataccgttgctcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacggtcacaacggcaaagtgttgccgcgggaatacctgaaagaagcatgggaatttacccgcgagcgcaatctggcgctgcatgttgacggggcgcacatctttaatgccgtggtggcttacggctgcgaactgaaagagatcacgcaatattgtgattcgttcaccatttgcctgtcgaaagggctggggactccagtcggtgagttactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaacatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 60 - protein, 333 aa, synthetic construct
  REGION 1..333: note = Variant of Threonine Aldolase from Escherichia coli
MIDLRSDTVARPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGAHIFNAVVAYGCELKEITQYCDSFTICLSKGLGTPVGELLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 61 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccctggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 62 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNALVAYGCELKEITQYCDSFTICLSKGLGTPVGSLLVGNRDYIKRAIRWRKMTGGGMRQSGILAAAGIYALKNNVARLQEDWDNAAWMAEQLREAGADVMRQDTNMLFVRVGEENAAALGEYMKARNVLINASPIVRLVTHLDVSREQLAEVAAHWRAFLAR

SEQ ID NO: 63 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttagtcctaccggcactcaagccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcctgtttaatgctgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 64 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFSPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARLFNAVVAYGCELKEITQYCDSFTICLSKGLGTPVGSLLVGNRDYIKRAIRWRKMTGGGMRQSGILAAAGIYALKNNVARLQEDWDNAAWMAEQLREAGADVMRQDTNMLFVRVGEENAAALGEYMKARNVLINASPIVRLVTHLDVSREQLAEVAAHWRAFLAR

SEQ ID NO: 65 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccattttttaccctaccggcactagcgccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttctgcgcttgggtgaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 66 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFYPTGTSANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVLRLGEDHDNAAWMAEQLREAGADVMRQDTNMLFVRVGEENAAALGEYMKARNVLINASPIVRLVTHLDVSREQLAEVAAHWRAFLAR

SEQ ID NO: 67 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccgcgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 68 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLQEDRDNAAWMAEQLREAGADVMRQDTNMLFVRVGEENAAALGEYMKARNVLINASPIVRLVTHLDVSREQLAEVAAHWRAFLAR

SEQ ID NO: 69 - DNA, 999 bp, synthetic construct
  misc_feature 1..999: note = Variant of Threonine Aldolase from Escherichia coli
  source 1..999: mol_type = other DNA
atgattgatttacgcagtgataccgttactcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaactgtcacaacggcaaagtgttgccgcgggaatacctgaaagaagcatgggaatttacccgcgagcgcaatctggcgctgcatgttgacatggcgcgcatctttaatgccgtggtggcttacggctgcgaactgaaagagatcacgcaatattgtgattcgttcaccatttgcctgtcgaaagggctggggactccagtcggttcgttactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaacatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 70 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENCHNGKVLPREYLKEAWEFTRERNLALHVDMARIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 71 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaagggctggggcctccagtcggtgagttactcgtcggtaatcgtgattacattaaacgtgccattcgctggcggaaaatgacaggtggcgggatgcgccagtccggcattctggctgccgccgggatatatgccctgaaaaataattttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 72 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGPPVGELLVGNRDYIKRAIRWRKMTGGGMRQSGILAAAGIYALKNNFARLQEDHDNAAWMAEQLREAGADVMRQDTNMLFVRVGEENAAALGEYMKARNVLINASPIVRLVTHLDVSREQLAEVAAHWRAFLAR

SEQ ID NO: 73 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaagccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgtttaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgggtgaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 74 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLGEDWDNAAWMAEQLREAGADVMRQDTNMLFVRVGEENAAALGEYMKARNVLINASPIVRLVTHLDVSREQLAEVAAHWRAFLAR

SEQ ID NO: 75 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaactgtcacaacggcaaagtgttgccgcgggaatacctgaaagaagcatgggaatttacccgcgagcgcaatctggcgctgcatgttgacatggcgcgcatctttaatgccgtggtggcttacggctgcgaactgaaagagatcacgcaatattgtgattcgttcaccatttgcctgtcgaaaggtcttgggacgccagtcggttcgttactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcctcttgacgtctcgcgcgaacaactggcggaagtcgccgcccactggcgtgcattcctggcgcgt

SEQ ID NO: 76 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENCHNGKVLPREYLKEAWEFTRERNLALHVDMARIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTPLDVSREQLAEVAAHWRAFLAR

SEQ ID NO: 77 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttactcctaccggcactagcgccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccgcgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 78 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFTPTGTSANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLQEDRDNAAWMAEQLREAGADVMRQDTNMLFVRVGEENAAALGEYMKARNVLINASPIVRLVTHLDVSREQLAEVAAHWRAFLAR

SEQ ID NO: 79 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatcgctaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 80 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIANAVVAYGCELKEITQYCDSFTICLSKGLGTPVGSLLVGNRDYIKRAIRWRKMTGGGMRQSGILAAAGIYALKNNVARLQEDWDNAAWMAEQLREAGADVMRQDTNMLFVRVGEENAAALGEYMKARNVLINASPIVRLVTHLDVSREQLAEVAAHWRAFLAR

SEQ ID NO: 81 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgcccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacatgggcaaatcgttgccgcgggaatacctgaaagaagcatgggaatttacccgcgagcgcaatctggcgctgcatgttgacggtgcgcgcatctttaatgccgtggtgctgtacggctgcgaactgaaagagatcacgcaatattgtgattcgttcaccatttgcctgtcgaaaggtcttgggacgccagtcggttcagccctcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 82 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHMGKSLPREYLKEAWEFTRERNLALHVDGARIFNAVVLYGCELKEITQYCDSFTICLSKGLGTPVGSALVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 83 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatactgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgggtgaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 84 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNTVVAYGCELKEITQYCDSFTICLSKGLGTPVGSLLVGNRDYIKRAIRWRKMTGGGMRQSGILAAAGIYALKNNVARLGEDWDNAAWMAEQLREAGADVMRQDTNMLFVRVGEENAAALGEYMKARNVLINASPIVRLVTHLDVSREQLAEVAAHWRAFLAR

SEQ ID NO: 85 - DNA, 999 bp, synthetic construct
  misc_feature 1..999: note = Variant of Threonine Aldolase from Escherichia coli
  source 1..999: mol_type = other DNA
atgattgatttacgcagtgatacggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcggagctggcgccgtggtggcttacggctgcgaactgaaagagatcacgcaatattgtgattcgttcaccactacgctgagtaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 86 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARGAGAVVAYGCELKEITQYCDSFTTTLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 87 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatactgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttctgcgcttgggtgaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcaccgcccactggcgtgcattcctggcgcgt

SEQ ID NO: 88 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNTVVAYGCELKEITQYCDSFTICLSKGLGTPVGSLLVGNRDYIKRAIRWRKMTGGGMRQSGILAAAGIYALKNNVLRLGEDHDNAAWMAEQLREAGADVMRQDTNMLFVRVGEENAAALGEYMKARNVLINASPIVRLVTHLDVSREQLAEVTAHWRAFLAR

SEQ ID NO: 89 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccattttttaccctaccggcactagcgccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgtttaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgggtgaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 90 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFYPTGTSANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLGEDWDNAAWMAEQLREAGADVMRQDTNMLFVRVGEENAAALGEYMKARNVLINASPIVRLVTHLDVSREQLAEVAAHWRAFLAR

SEQ ID NO: 91 - DNA, 999 bp, synthetic construct
  misc_feature 1..999: note = Variant of Threonine Aldolase from Escherichia coli
  source 1..999: mol_type = other DNA
atgattgatttacgcagtgataccgttactcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggggcgcacatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 92 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGAHIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 93 - DNA, 999 bp, synthetic construct
  misc_feature 1..999: note = Variant of Threonine Aldolase from Escherichia coli
  source 1..999: mol_type = other DNA
atgattgatttacgcagtgataccgttaccctgccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcagccctcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 94 - protein, 333 aa, synthetic construct
  REGION 1..333: note = Variant of Threonine Aldolase from Escherichia coli
MIDLRSDTVTLPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSALVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 95 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcactttggcaaatcgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcagccctcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 96 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHFGKSLPREYLKEAWEFTRERNLALHVDGARIFNAVVAYGCELKEITQYCDSFTICLSKGLGTPVGSALVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 97 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcgtatttaacgccgtggtggcttacggctgcgaactgaaagagatcacgcaatattgtgattcgttcaccatttgtctgcataaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 98 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARVFNAVVAYGCELKEITQYCDSFTICLHKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 99 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccattttttaccctaccggcactagcgccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatcggtaatgccctggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 100 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFYPTGTSANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIGNALVAYGCELKEITQYCDSFTICLSKGLGTPVGSLLVGNRDYIKRAIRWRKMTGGGMRQSGILAAAGIYALKNNVARLQEDWDNAAWMAEQLREAGADVMRQDTNMLFVRVGEENAAALGEYMKARNVLINASPIVRLVTHLDVSREQLAEVAAHWRAFLAR

SEQ ID NO: 101 - DNA, 999 bp, synthetic construct
  misc_feature 1..999: note = Variant of Threonine Aldolase from Escherichia coli
  source 1..999: mol_type = other DNA
atgattgatttacgcagtgatacggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatttttggcgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 102 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFGAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 103 - DNA, 999 bp, synthetic construct
  misc_feature 1..999: note = Variant of Threonine Aldolase from Escherichia coli
  source 1..999: mol_type = other DNA
atgattgatttacgcagtgataccgttactcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgctcacaacggcaaagtgttgccgcgggaatacctgaaagaagcatgggaatttacccgcgagcgcaatctggcgctgcatgttgacatggcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 104 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENAHNGKVLPREYLKEAWEFTRERNLALHVDMARIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 105 - DNA, 999 bp, synthetic construct
  misc_feature 1..999: note = Variant of Threonine Aldolase from Escherichia coli
  source 1..999: mol_type = other DNA
atgattgatttacgcagtgatacggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcattgctggcgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 106 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIAGAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 107 - DNA, 999 bp, synthetic construct
  misc_feature 1..999: note = Variant of Threonine Aldolase from Escherichia coli
  source 1..999: mol_type = other DNA
atgattgatttacgcagtgatacggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccactacgctgagtaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 108 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTTTLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 109 - DNA, 999 bp, synthetic construct
  misc_feature 1..999: note = Variant of Threonine Aldolase from Escherichia coli
  source 1..999: mol_type = other DNA
atgattgatttacgcagtgataccgttaccctgccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcagctggcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcagccctcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 110 - protein, 333 aa, synthetic construct
  REGION 1..333: note = Variant of Threonine Aldolase from Escherichia coli
MIDLRSDTVTLPSRAMLEAMMAAPVGDDVYGDDPTVNALQDYAAELSGKEAAIFLPTGTQANLVALLSHCERGEEYIVGQLAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSALVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 111 - DNA, 999 bp, synthetic construct
  misc_feature 1..999: note = Variant of Threonine Aldolase from Escherichia coli
  source 1..999: mol_type = other DNA
atgattgatttacgcagtgataccgttactcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataatgttgcgcgcttgcaggaagaccacgacaacgctgcctggatggcggagcagctgcgtgaagcaggcgcggatgtgatgcgtcaggacacccacatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 112 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTHMLFVRVGEENAAALGEYMKARNVLINASPIVRLVTHLDVSREQLAEVAAHWRAFLAR

SEQ ID NO: 113 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatcgctaatgccctggtggcttacggctgcgaactgaaagagatcacgcaatattgtgattcgttcaccatttgcctgtcgaaaggtcttgggtgcccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccgcgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 114 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIANALVAYGCELKEITQYCDSFTICLSKGLGCPVGSLLVGNRDYIKRAIRWRKMTGGGMRQSGILAAAGIYALKNNVARLQEDRDNAAWMAEQLREAGADVMRQDTNMLFVRVGEENAAALGEYMKARNVLINASPIVRLVTHLDVSREQLAEVAAHWRAFLAR

SEQ ID NO: 115 - DNA, 999 bp, synthetic construct
  misc_feature 1..999: note = Variant of Threonine Aldolase from Escherichia coli
  source 1..999: mol_type = other DNA
atgattgatttacgcagtgatacggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcattgctaacgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 116 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIANAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 117 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcggccaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagactgggacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 118 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSATICLSKGLGTPVGSLLVGNRDYIKRAIRWRKMTGGGMRQSGILAAAGIYALKNNVARLQEDWDNAAWMAEQLREAGADVMRQDTNMLFVRVGEENAAALGEYMKARNVLINASPIVRLVTHLDVSREQLAEVAAHWRAFLAR

SEQ ID NO: 119 - DNA, 999 bp, synthetic construct
  misc_feature 1..999: note = Variant of Threonine Aldolase from Escherichia coli
  source 1..999: mol_type = other DNA
atgattgatttacgcagtgataccgttactcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgctcacaacggcaaagtgttgccgcgggaatacctgaaagaagcatgggaatttacccgcgagcgcaatctggcgctgcatgttgacggggcgcacatctttaatgccgtggtggcttacggctgcgaactgaaagagatcacgcaatattgtgattcgttcaccatttgcctgtcgaaagggctggggactccagtcggtgagttactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 120 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENAHNGKVLPREYLKEAWEFTRERNLALHVDGAHIFNAVVAYGCELKEITQYCDSFTICLSKGLGTPVGELLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 121 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgcccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaatggcaaagtattgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcagccctcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 122 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSALVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 123 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctagcggcactcaggccaacctggtcgctctgctcagtcactgcgaacgcggcgaagagtatattgtcggtcaggctgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcactttggcaaatcgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaggtcttgggacgccagtcggttcagccctcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 124 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPSGTQANLVALLSHCERGEEYIVGQAAHNYLFEAGGAAVLGSIQPQPIDAAADGTLPLDKVAMKIKPDDIHFARTKLLSLENGHFGKSLPREYLKEAWEFTRERNLALHVDGARIFNAVVAYGCELKEITQYCDSFTICLSKGLGTPVGSALVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 125 - DNA, 999 bp, synthetic construct
  misc_feature 1..999: note = Variant of Threonine Aldolase from Escherichia coli
  source 1..999: mol_type = other DNA
atgattgatttacgcagtgataccgttgctcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggggcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 126 - protein, 333 aa, synthetic construct
  REGION 1..333: note = Variant of Threonine Aldolase from Escherichia coli
MIDLRSDTVARPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 127 - DNA, 999 bp, synthetic construct
  misc_feature 1..999: note = Variant of Threonine Aldolase from Escherichia coli
  source 1..999: mol_type = other DNA
atgattgatttacgcagtgatacggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcgtatttaacgccgtggtggcttacggctgcgaactgaaagagatcacgcaatattgtgattcgttcaccacttgtactcataaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 128 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARVFNAVVAYGCELKEITQYCDSFTTCTHKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 129 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaactgtcacaacggcaaagtgttgccgcgggaatacctgaaagaagcatgggaatttacccgcgagcgcaatctggcgctgcatgttgacggggcgcacatctttaatgccgtggtggcttacggctgcgaactgaaagagatcacgcaatattgtgattcgttcaccatttgcctgtcgaaagggctggggactccagtcggttcgttactcgtcggtaatcgtgattacattaaacgtgccattcgctggcggaaaatgacaggtggcgggatgcgccagtccggcattctggctgccgccgggatatatgccctgaaaaataatgttgcgcgcttgcaggaagaccacgacaacgctgcctggatggcggagcagctgcgtgaagcaggcgcggatgtgatgcgtcaggacacccacatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 130 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENCHNGKVLPREYLKEAWEFTRERNLALHVDGAHIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTHMLFVRVGEENAAALGEYMKARNVLINASPIVRLVTHLDVSREQLAEVAAHWRAFLAR

SEQ ID NO: 131 - DNA, 999 bp, synthetic construct
  misc_feature 1..999: note = Variant of Threonine Aldolase from Escherichia coli
  source 1..999: mol_type = other DNA
atgattgatttacgcagtgagacggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcgtagctggcgccgtggtggcttacggctgcgaactgaaagagatcacgcaatattgtgattcgttcaccatttgtactcataaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcctgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 132 - protein, 333 aa, synthetic construct
  REGION 1..333: note = Variant of Threonine Aldolase from Escherichia coli
MIDLRSETVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARVAGAVVAYGCELKEITQYCDSFTICTHKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 133 - DNA, 999 bp, synthetic construct
  misc_feature 1..999: note = Variant of Threonine Aldolase from Escherichia coli
  source 1..999: mol_type = other DNA
atgattgatttacgcagtgataccgttgctcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaactgtcacaacggcaaagtgttgccgcgggaatacctgaaagaagcatgggaatttacccgcgagcgcaatctggcgctgcatgttgacggggcgcgcatctttaatgccgtggtggcttacggctgcgaactgaaagagatcacgcaatattgtgattcgttcaccatttgcctgtcgaaaggtcttgggacgccagtcggtgagttactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcctcttgacgtctcgcgcgaacaactggcggaagtcgccgcccactggcgtgcattcctggcgcgt

SEQ ID NO: 134 - protein, 333 aa, synthetic construct
  REGION 1..333: note = Variant of Threonine Aldolase from Escherichia coli
MIDLRSDTVARPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENCHNGKVLPREYLKEAWEFTRERNLALHVDGARIFNAVVAYGCELKEITQYCDSFTICLSKGLGTPVGELLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTPLDVSREQLAEVAAHWRAFLAR

SEQ ID NO: 135 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatttttaacgccgtggtggcttacggctgcgaactgaaagagatcacgcaatattgtgattcgttcaccatttgtactagtaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 136 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICTSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 137 - DNA, 999 bp, synthetic construct
  misc_feature 1..999: note = Variant of Threonine Aldolase from Escherichia coli
  source 1..999: mol_type = other DNA
atgattgatttacgcagtgataccgttactcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaactgtcacaacggcaaagtgttgccgcgggaatacctgaaagaagcatgggaatttacccgcgagcgcaatctggcgctgcatgttgacggggcgcgcatctttaatgccgtggtggcttacggctgcgaactgaaagagatcacgcaatattgtgattcgttcaccatttgcctgtcgaaagggctggggactccagtcggttcgttactcgtcggtaatcgtgattacattaaacgtgccattcgctggcggaaaatgacaggtggcgggatgcgccagtccggcattctggctgccgccgggatatatgccctgaaaaatgaggttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 138 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENCHNGKVLPREYLKEAWEFTRERNLALHVDGARIFNAVVAYGCELKEITQYCDSFTICLSKGLGTPVGSLLVGNRDYIKRAIRWRKMTGGGMRQSGILAAAGIYALKNEVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 139 - DNA, 999 bp, synthetic construct
  misc_feature 1..999: note = Variant of Threonine Aldolase from Escherichia coli
  source 1..999: mol_type = other DNA
atgattgatttacgcagtgatctggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcagttttaacgccgtggtggcttacggctgcgaactgaaagagatcacgcaatattgtgattcgttcaccattacgctgcataaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 140 - protein, 333 aa, synthetic construct
  REGION 1..333: note = Variant of Threonine Aldolase from Escherichia coli
MIDLRSDLVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARSFNAVVAYGCELKEITQYCDSFTITLHKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 141 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacatggcgcgcatctttaatgccgtggtggcttacggctgcgaactgaaagagatcacgcaatattgtgattcgttcaccatttgcctgtcgaaaggtcttgggactccagtcggtgagttactcgtcggtaatcgtgattacattaaacgtgccattcgctggcggaaaatgacaggtggcgggatgcgccagtccggcattctggctgccgccgggatatatgccctgaaaaataatgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 142 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDMARIFNAVVAYGCELKEITQYCDSFTICLSKGLGTPVGELLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 143 - DNA, 999 bp, synthetic construct
  misc_feature 1..999: note = Variant of Threonine Aldolase from Escherichia coli
  source 1..999: mol_type = other DNA
atgattgatttacgcagtgatacggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccacttgtctgagtaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 144 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTTCLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 145 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcagttttggcgccgtggtggcttacggctgcgaactgaaagagatcacgcaatattgtgattcgttcaccatttgtctgagtaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 146 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARSFGAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 147 - DNA, 999 bp, synthetic construct
  misc_feature 1..999: note = Variant of Threonine Aldolase from Escherichia coli
  source 1..999: mol_type = other DNA
atgattgatttacgcagtgataccgttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatttttaacgccgtggtggcttacggctgcgaactgaaagagatcacgcaatattgtgattcgttcaccatttgtctgcataaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 148 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLHKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 149 - DNA, 999 bp, synthetic construct
  misc_feature 1..999: note = Variant of Threonine Aldolase from Escherichia coli
  source 1..999: mol_type = other DNA
atgattgatttacgcagtgagacggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcagtgctaacgccgtggtggcttacggctgcgaactgaaagagatcacgcaatattgtgattcgttcaccacttgtctgagtaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 150 - protein, 333 aa, synthetic construct
  REGION 1..333: note = Variant of Threonine Aldolase from Escherichia coli
MIDLRSETVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARSANAVVAYGCELKEITQYCDSFTTCLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 151 - DNA, 999 bp, synthetic construct
  misc_feature 1..999: note = Variant of Threonine Aldolase from Escherichia coli
  source 1..999: mol_type = other DNA
atgattgatttacgcagtgataccgttactcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacggtgcgcgcatctttaatgccgtggtggcttacggctgcgaactgaaagagatcacgcaatattgtgattcgttcaccatttgcctgtcgaaaaccctggggcctccagtcggtgagttactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 152 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTICLSKTLGPPVGELLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 153 - DNA, 999 bp, synthetic construct
  misc_feature 1..999: note = Variant of Threonine Aldolase from Escherichia coli
  source 1..999: mol_type = other DNA
atgattgatttacgcagtgataccgttgctcgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcatgggaatttacccgcgagcgcaatctggcgctgcatgttgacatggcgcgcatctttaatgccgtggtggcttacggctgcgaactgaaagagatcacgcaatattgtgattcgttcaccatttgcctgtcgaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 154 - protein, 333 aa, synthetic construct
  REGION 1..333: note = Variant of Threonine Aldolase from Escherichia coli
MIDLRSDTVARPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDMARIFNAVVAYGCELKEITQYCDSFTICLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 155 - DNA, 999 bp, synthetic construct
  misc_feature 1..999: note = Variant of Threonine Aldolase from Escherichia coli
  source 1..999: mol_type = other DNA
atgattgatttacgcagtgatacggttacccgaccaagccgcgccatgctcgaagcgatgatggccgccccggttggggacgacgtttacggagacgaccctaccgttaatgctctgcaggactacgcagcagagctttccggtaaagaagccgccatttttctgcctaccggcactcaggccaacctggtcgctctgctcagtcactgcgaacgcggcgaagagtatattgtcggtcaggccgcgcataactatctgtttgaagccggtggcgcggcggtgctgggcagtattcaaccgcaacccatagacgcggctgccgacggcacgctaccgctggataaagtggcgatgaaaatcaaacccgacgatatccatttcgcccgcaccaaattactcagtctggaaaacgggcacaacggcaaagtgttgccgcgggaatacctgaaagaagcctgggaatttacccgcgagcgcaatctggcgctgcatgttgacggtgcgcgcatctttaatgccgtggtggcttacggctgcgaactgaaagagatcacgcaatattgtgattcgttcaccattacgctgagtaaaggtcttgggacgccagtcggttcattactcgtcggtaatcgtgattacattaaacgtgccattcgctggcggaaaatgacaggtggcgggatgcgccagtccggcattctggctgccgccgggatatatgccctgaaaaataacgttgcgcgcttgcaggaagaccacgacaacgctgcctggatggcggagcagctgcgtgaagcaggcgcggatgtgatgcgtcaggacaccaatatgctgtttgttcgcgtcggggaagaaaatgctgccgcgttaggcgaatacatgaaagcgagaaacgtgctgattaacgcctcgccgattgtccgcctggtgacgcatcttgacgtctcgcgcgaacaactggcggaagtcgccgcccactggcgtgcattcctggcgcgt

SEQ ID NO: 156 - protein, 333 aa, synthetic construct
  REGION 1..333: note = Variant of Threonine Aldolase from Escherichia coli
MIDLRSDTVTRPSRAMLEAMMAAPVGDDVYGDDPTVNALQDYAAELSGKEAAIFLPTGTQANLVALLSHCERGEEYIVGQAAHNYLFEAGGAAVLGSIQPQPIDAAADGTLPLDKVAMKIKPDDIHFARTKLLSLENGHNGKVLPREYLKEAWEFTRERNLALHVDGARIFNAVVAYGCELKEITQYCDSFTITLSKGLGTPVGSLLVGNRDYIKRAIRWRKMTGGGMRQSGILAAAGIYALKNNVARLQEDHDNAAWMAEQLREAGADVMRQDTNMLFVRVGEENAAALGEYMKARNVLINASPIVRLVTHLDVSREQLAEVAAHWRAFLAR

SEQ ID NO: 157 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 158 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 159 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgaaagattaccaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 160 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLMKDYQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 161 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgaaagattaccaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 162 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLMKDYQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 163 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgacgaagggtggcacggaaaatgggtgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 164 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLTKGGTENGCWCAEALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 165 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgttggtgccgcgaggccctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 166 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGCWCREALVLFDPSRAQEMHFLRKRSAQLFSKSRFVAAQFDAYLAGDLMLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 167 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatatggcgcattttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagaaagatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 168 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDMAHFANALVSLGTTPAEMTWKRGIDLLSFGGTKNGCWCAEALVLFDPSRAQEMHFLRKRSAQLFSKSRFVAAQKDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 169 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgacgaagggtggctcgaaaaatctgtgctggtgcgcggaggcgctggtgctgtttgatccgagccgtgcgcaggaaatgcacttcctgcgtaagcgtagcgcgcaactgttcagcaaaagccgttttgttgcggcgcagtttgatgcggcactggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 170 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLTKGGSKNLCWCAEALVLFDPSRAQEMHFLRKRSAQLFSKSRFVAAQFDAALAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 171 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctgcaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 172 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLWLDLQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 173 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggtatgtggtgcgcgagcgccctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggattaccaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 174 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGMWCASALVLFDPSRAQEMHFLRKRSAQLFSKSRFVAAQFDAYLAGDLWLDYQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 175 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggtatgtggtgcccgcaggccctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggaaagattacgcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 176 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGMWCPQALVLFDPSRAQEMHFLRKRSAQLFSKSRFVAAQFDAYLAGDLWKDYARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 177 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggggcgcgtattgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagaaagatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 178 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARIANALVSLGTTPAEMTWKRGIDLLSFGGTKNGCWCAEALVLFDPSRAQEMHFLRKRSAQLFSKSRFVAAQKDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 179 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggaaagattaccaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 180 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLWKDYQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 181 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggtatgtggtgccgcgagggcctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggattacgcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 182 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGMWCREGLVLFDPSRAQEMHFLRKRSAQLFSKSRFVAAQFDAYLAGDLWLDYARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 183 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggtctgtggtgcgcggagggcctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggaaagatctgcaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 184 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGLWCAEGLVLFDPSRAQEMHFLRKRSAQLFSKSRFVAAQFDAYLAGDLWKDLQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 185 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctgtcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 186 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLWLDLSRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 187 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgctggattaccaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 188 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLMLDYQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 189 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgctggatctgcaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 190 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLMLDLQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 191 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatatggcgcatgttgcgaacgcgctggttagcctgggtaccaccccggcggagatgacctggaagcgtggtatcgacctgctgtcgaagggtggctcgaaagaggggtgctggtgcgcggaggcgctggtgctgtttgatccgagccgtgcgcaggaaatgcacttcctgcgtaagcgtagcgcgcaactgttcagcaaaagccgttttgttgcggcgcagaaagatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 192 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDMAHVANALVSLGTTPAEMTWKRGIDLLSKGGSKEGCWCAEALVLFDPSRAQEMHFLRKRSAQLFSKSRFVAAQKDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 193 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatatggcgcatgttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagaaagatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 194 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDMAHVANALVSLGTTPAEMTWKRGIDLLSFGGTKNGCWCAEALVLFDPSRAQEMHFLRKRSAQLFSKSRFVAAQKDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 195 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgacgaagggtggctcggaaaatctgtgctggtgcgcggaggcgctggtgctgtttgatccgagccgtgcgcaggaaatgcacttcctgcgtaagcgtagcgcgcaactgttcagcaaaagccgttttgttgcggcgcagaaagatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 196 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLTKGGSENLCWCAEALVLFDPSRAQEMHFLRKRSAQLFSKSRFVAAQKDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 197 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggtatgtggtgcgcgggggccctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 198 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGMWCAGALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 199 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgctggattacgcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 200 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLMLDYARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 201 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggggcgcatattgcgaacgcgctggttagcctgggtaccaccccggcggagatgacctggaagcgtggtatcgacctgctgagcttcggtggcaccaaaaacgggtgctggtgcgcggaggcgctggtgctgtttgatccgagccgtgcgcaggaaatgcacttcctgcgtaagcgtagcgcgcaactgttcagcaaaagccgttttgttgcggcgcagaaagatgcggcactggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 202 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGAHIANALVSLGTTPAEMTWKRGIDLLSFGGTKNGCWCAEALVLFDPSRAQEMHFLRKRSAQLFSKSRFVAAQKDAALAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 203 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgacgaagggtggcacgaaaaatctgtgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 204 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLTKGGTKNLCWCAEALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 205 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatatggcgcgtgttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 206 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDMARVANALVSLGTTPAEMTWKRGIDLLSFGGTKNGCWCAEALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 207 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgttggtgcgcagcggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgctggatctgtcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 208 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGCWCAAALVLFDPSRAQEMHFLRKRSAQLFSKSRFVAAQFDAYLAGDLMLDLSRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 209 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggggcgcatattgcgaacgcgctggttagcctgggtaccaccccggcggagatgacctggaagcgtggtatcgacctgctgacgaagggtggctcgaaaaatctgtgctggtgcgcggaggcgctggtgctgtttgatccgagccgtgcgcaggaaatgcacttcctgcgtaagcgtagcgcgcaactgttcagcaaaagccgttttgttgcggcgcagaaagatgcggcactggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 210 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGAHIANALVSLGTTPAEMTWKRGIDLLTKGGSKNLCWCAEALVLFDPSRAQEMHFLRKRSAQLFSKSRFVAAQKDAALAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 211 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggattaccaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 212 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLWLDYQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 213 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggggcgcatattgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagaaagatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 214 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGAHIANALVSLGTTPAEMTWKRGIDLLSFGGTKNGCWCAEALVLFDPSRAQEMHFLRKRSAQLFSKSRFVAAQKDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 215 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagaaagatgcggcactggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 216 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQKDAALAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 217 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggtatgtggtgcgcggggggcctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgaaagattaccaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 218 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGMWCAGGLVLFDPSRAQEMHFLRKRSAQLFSKSRFVAAQFDAYLAGDLMKDYQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 219 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgttggtgcgcgagcgccctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgctggattaccaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 220 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGCWCASALVLFDPSRAQEMHFLRKRSAQLFSKSRFVAAQFDAYLAGDLMLDYQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 221 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgacgtttggtggctcggaaaatgggtgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 222 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLTFGGSENGCWCAEALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 223 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggggcgcatattgcgaacgcgctggttagcctgggtaccaccccggcggagatgacctggaagcgtggtatcgacctgctgacgaagggtggcacgaaagaggggtgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 224 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGAHIANALVSLGTTPAEMTWKRGIDLLTKGGTKEGCWCAEALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 225 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggagggcctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgctggattactcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 226 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGCWCAEGLVLFDPSRAQEMHFLRKRSAQLFSKSRFVAAQFDAYLAGDLMLDYSRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 227 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatatggcgcatgttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 228 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDMAHVANALVSLGTTPAEMTWKRGIDLLSFGGTKNGCWCAEALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 229 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgacgtttggtggctcggaaaatctgtgctggtgcgcggaggcgctggtgctgtttgatccgagccgtgcgcaggaaatgcacttcctgcgtaagcgtagcgcgcaactgttcagcaaaagccgttttgttgcggcgcagaaagatgcggcactggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 230 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLTFGGSENLCWCAEALVLFDPSRAQEMHFLRKRSAQLFSKSRFVAAQKDAALAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 231 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgttggtgcgcggagggcctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggattaccaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 232 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGCWCAEGLVLFDPSRAQEMHFLRKRSAQLFSKSRFVAAQFDAYLAGDLWLDYQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 233 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgtcgtttggtggctcgaaaaatgggtgctggtgcgcggaggcgctggtgctgtttgatccgagccgtgcgcaggaaatgcacttcctgcgtaagcgtagcgcgcaactgttcagcaaaagccgttttgttgcggcgcagaaagatgcggcactggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 234 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGSKNGCWCAEALVLFDPSRAQEMHFLRKRSAQLFSKSRFVAAQKDAALAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 235 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgaaagatctgcaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 236 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLMKDLQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 237 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggggcgcattttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagaaagatgcggcactggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 238 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGAHFANALVSLGTTPAEMTWKRGIDLLSFGGTKNGCWCAEALVLFDPSRAQEMHFLRKRSAQLFSKSRFVAAQKDAALAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 239 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgccgcgagggcctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctgcaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 240 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGCWCREGLVLFDPSRAQEMHFLRKRSAQLFSKSRFVAAQFDAYLAGDLWLDLQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 241 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgggctggttagcctgggtaccaccccggcggagatgacctggaagcgtggtatcgacctgctgagcttcggtggcaccaaaaacggtctgtggtgcgcgagcgccctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgaaagattactcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 242 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANGLVSLGTTPAEMTWKRGIDLLSFGGTKNGLWCASALVLFDPSRAQEMHFLRKRSAQLFSKSRFVAAQFDAYLAGDLMKDYSRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 243 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatatggcgcattttgcgaacgcgctggttagcctgggtaccaccccggcggagatgacctggaagcgtggtatcgacctgctgacgaagggtggctcgaaagaggggtgctggtgcgcggaggcgctggtgctgtttgatccgagccgtgcgcaggaaatgcacttcctgcgtaagcgtagcgcgcaactgttcagcaaaagccgttttgttgcggcgcagtttgatgcggcactggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 244 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDMAHFANALVSLGTTPAEMTWKRGIDLLTKGGSKEGCWCAEALVLFDPSRAQEMHFLRKRSAQLFSKSRFVAAQFDAALAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 245 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatatggcgcgttttgcgaacgcgctggttagcctgggtaccaccccggcggagatgacctggaagcgtggtatcgacctgctgacgtttggtggctcggaaaatctgtgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 246 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDMARFANALVSLGTTPAEMTWKRGIDLLTFGGSENLCWCAEALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 247 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgaaagatctgcaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 248 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLMKDLQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 249 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgggctggtttgcctgggtaccaccccggcggagatgacctggaagcgtggtatcgacctgctgagcttcggtggcaccaaaaacggttgttggtgcgcgagcgccctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgaaagatctgcaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 250 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANGLVCLGTTPAEMTWKRGIDLLSFGGTKNGCWCASALVLFDPSRAQEMHFLRKRSAQLFSKSRFVAAQFDAYLAGDLMKDLQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 251 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggggcgcatgttgcgaacgcgctggttagcctgggtaccaccccggcggagatgacctggaagcgtggtatcgacctgctgacgtttggtggctcggaagaggggtgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 252 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGAHVANALVSLGTTPAEMTWKRGIDLLTFGGSEEGCWCAEALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 253 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggtatgtggtgcgcgagcgccctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 254 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGMWCASALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 255 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggggcgcgttcgcaaaacgcgctggttagcctgggtaccaccccggcggagatgacctggaagcgtggtatcgacctgctgacgtttggtggcacgaaaaatgggtgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 256 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARSQNALVSLGTTPAEMTWKRGIDLLTFGGTKNGCWCAEALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 257 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggtatgtggtgccgcgaggccctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgctggattaccaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 258 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGMWCREALVLFDPSRAQEMHFLRKRSAQLFSKSRFVAAQFDAYLAGDLMLDYQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 259 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgttggtgcgcgagcgccctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggattaccaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 260 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGCWCASALVLFDPSRAQEMHFLRKRSAQLFSKSRFVAAQFDAYLAGDLWLDYQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 261 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggtctgtggtgccgcgaggccctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgctggatctgcaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 262 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGLWCREALVLFDPSRAQEMHFLRKRSAQLFSKSRFVAAQFDAYLAGDLMLDLQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 263 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatatggcgcatattgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagaaagatgcggcactggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 264 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDMAHIANALVSLGTTPAEMTWKRGIDLLSFGGTKNGCWCAEALVLFDPSRAQEMHFLRKRSAQLFSKSRFVAAQKDAALAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 265 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggtatgtggtgccgcgaggccctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggaaagatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 266 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGMWCREALVLFDPSRAQEMHFLRKRSAQLFSKSRFVAAQFDAYLAGDLWKDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 267 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcgagcgccctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggattaccaacgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 268 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGCWCASALVLFDPSRAQEMHFLRKRSAQLFSKSRFVAAQFDAYLAGDLWLDYQRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 269 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggtctgtggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggaaagatctgtcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 270 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGLWCAEALVLFDPSRAQEMHFLRKRSAQLFSKSRFVAAQFDAYLAGDLWKDLSRHANAMARRLADGITASAESRLAWAPDANEVFVVLKREAASRLRQQGALFYDWEVPHDLEGSLAEDEGLFRLVTSFATRAEDVDRFVAAC

SEQ ID NO: 271 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatatggcgcattttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcggcactggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 272 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDMAHFANALVSLGTTPAEMTWKRGIDLLSFGGTKNGCWCAEALVLFDPSRAQEMHFLRKRSAQLFSKSRFVAAQFDAALAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 273 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacctcggcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgttggtgcgcggggggcctggtgctgtttgatccgagccgtgcgcaggaaatgcacttcctgcgtaagcgtagcgcgcaactgttcagcaaaagccgttttgttgcggcgcagtttgatgcgtacctggcgggtgacctgatgctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 274 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTSATESGTVYSLGEIEAIASIARSQTLPLHMDGARFANALVSLGTTPAEMTWKRGIDLLSFGGTKNGCWCAGGLVLFDPSRAQEMHFLRKRSAQLFSKSRFVAAQFDAYLAGDLMLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 275 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggggcgcatgttgcgaacgcgctggttagcctgggtaccaccccggcggagatgacctggaagcgtggtatcgacctgctgagcttcggtggcaccaaaaatgggtgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 276 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGAHVANALVSLGTTPAEMTWKRGIDLLSFGGTKNGCWCAEALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 277 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctatagcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgcggcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggaggtcgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtctggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgatctggaaggtagcctggcggaggatgaaggcctgtttcgtctggttaccagcttcgcgacccgtgcggaagacgtggatcgtttcgttgcggcgtgc

SEQ ID NO: 278 - protein, 348 aa, synthetic construct
  REGION 1..348: note = Variant of Threonine Aldolase from Sinorhizobium arboris
MIFSSDNWAGAHPAIAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKEVVHGGQPMAVTLTQATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLWLDLARHANAMARRLADGITASAESRLAWAPDANEVFVVLKREAASRLRQQGALFYDWEVPHDLEGSLAEDEGLFRLVTSFATRAEDVDRFVAAC

SEQ ID NO: 279 - DNA, 1044 bp, synthetic construct
  misc_feature 1..1044: note = Variant of Threonine Aldolase from Sinorhizobium arboris
  source 1..1044: mol_type = other DNA
atgatattttcaagtgataattgggctggagcacaccccgctctggcagagagcctggtgacccatgcgaagggttacgcgagcgcgtatggcaccagcgaactggaccgtaaagtggaggaacgtttcagcgaggtttttgaacgtgatgtggcggttttctttgttggtaccggtaccgctgcgaacagcctggcgctgagcattgcgaaccgtgcgggtggcatcgcgttttgccaccgtgaggcgcacgtgaacaaggacgaatgcggtgcgccgcaattctttagccatggtgcgcgtctgagcgcggtgggtggcgcgcgtggcaagatggatccggcgaaactggaggcggaaattcgtcgtttcccgaaggagaacgtgcacggtggccagccgatggcggttaccctgacccaagcgaccgaaagcggtaccgtgtacagcctgggcgagatcgaagcgattgcgagcattgcgcgtagccaaaccctgccgctgcacatggatggtgcgcgttttgcgaacgcgctggttagcctgggtaccaccccggcggagatgacctggaagcgtggtatcgacctgctgagcttcggtggcaccaaaaacggttgctggtgcgcggaggcgctggtgctgtttgatccgagccgtgcgcaggaaatgcacttcctgcgtaagcgtagcgcgcaactgttcagcaaaagccgttttgttgcggcgcagtttgatgcgtacctggcgggtgacctgtggctggatctggcgcgtcatgcgaacgcgatggcgcgtcgtcaggcggacggtattaccgcgagcgcggagagccgtctggcgtgggcgccggatgcgaacgaggtttttgtggttctgaaacgtgaagcggcgagccgtctgcgtcagcaaggtgcgctgttctatgactgggaagtgccgcacgattgggaaggtagcctggcggaggatgaaggcctgtttcgtctggttaccagcttcgcgacccgtgcggaagacgtggatcgtttcgttgcggcgtgc

SEQ ID NO: 280 - protein, 348 aa, synthetic construct
  REGION 1..348: note = Variant of Threonine Aldolase from Sinorhizobium arboris
MIFSSDNWAGAHPALAESLVTHAKGYASAYGTSELDRKVEERFSEVFERDVAVFFVGTGTAANSLALSIANRAGGIAFCHREAHVNKDECGAPQFFSHGARLSAVGGARGKMDPAKLEAEIRRFPKENVHGGQPMAVTLTQATESGTVYSLGEIEAIASIARSQTLPLHMDGARFANALVSLGTTPAEMTWKRGIDLLSFGGTKNGCWCAEALVLFDPSRAQEMHFLRKRSAQLFSKSRFVAAQFDAYLAGDLWLDLARHANAMARRQADGITASAESRLAWAPDANEVFVVLKREAASRLRQQGALFYDWEVPHDWEGSLAEDEGLFRLVTSFATRAEDVDRFVAAC

SEQ ID NO: 281 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 282 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 283 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcagtatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 284 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCSIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 285 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtcctggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 286 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRPGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 287 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgtggccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 288 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPWPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 289 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccaattatcgcgtgcatacccgtgaaatcgaacgcgaggttctggcgtggttcgccgaactctttcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggggattcacgaaagccatatccattgtgacgcagcattagcggggatgaccctgccctttatggatgatgccccagcgtttgactttgccactggcattgattcgcttagcgtttccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgctcgattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 290 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASNYRVHTREIEREVLAWFAELFHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLGIHESHIHCDAALAGMTLPFMDDAPAFDFATGIDSLSVSGHKFIGSPIPCGIVLTRKHYVERIARSIEYVGTLDTTITGSRNGITPLFLWYAVKQAGRDGFKRRVAACLDNARYTVEQFKRIGVEARRNPCSITVVFPKPPQSVLHKWQIAVNGDRAHIILMPSTTLQHVDELIADIAAANKAAAAPSDTQKPAAKKRPTPAGAPQRTT

SEQ ID NO: 291 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactctttcacattgcaccagaagaatactggggttatgtgactaatggaggaaccgagggcaacctgtacggtttgtttttagcgcgtgaacgttttcctacgggcgtcgcctactattcggaagacacccattatagcgtgtataagaatctgcgtctgctgaacatgcgtcacatcatgatcaaatcacagccgaatggtgagattgattacggcgatctctacgaaactattcgcattcatcgtgattgcccgccgatcatcttcgctaacattggtacgacgatgaccgaaggcattgacgacattcgcaccatccaggatatgttcaagaaactggcgattcacgaatattatatccattgtgacgcagcattagcggggatgaccctgccctttatggatgatgccccagcgtttgactttgccactggcattgattcgcttagcgtttccggtcacaaattcattgggtctccgattccttgcggcatcgttttgactcgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgctcgattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 292 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELFHIAPEEYWGYVTNGGTEGNLYGLFLARERFPTGVAYYSEDTHYSVYKNLRLLNMRHIMIKSQPNGEIDYGDLYETIRIHRDCPPIIFANIGTTMTEGIDDIRTIQDMFKKLAIHEYYIHCDAALAGMTLPFMDDAPAFDFATGIDSLSVSGHKFIGSPIPCGIVLTRKHYVERIARSIEYVGTLDTTITGSRNGITPLFLWYAVKQAGRDGFKRRVAACLDNARYTVEQFKRIGVEARRNPCSITVVFPKPPQSVLHKWQIAVNGDRAHIILMPSTTLQHVDELIADIAAANKAAAAPSDTQKPAAKKRPTPAGAPQRTT

SEQ ID NO: 293 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcgataacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 294 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVDNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 295 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaattcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcctgtactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgctgaagaaactggcgattcacgaatctcatatccattgtgacgcagcattagcggggatgaccctgccctttatggatgatgccccagcgtttgactttgccactggcattgattcgatcagcatctccggtcacaaattcattgggtctccgattccttgcggcatcgttttgacaaagaaacattatgtggagcgtattgctcgcagtatcgaatatgtcggtaccctggacactacgattaccggcagccgtaatggtatcacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 296 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREFEREVLAWFAELMHIAPEEYWGYVTNGGTEGNLYGLFLARERFPTGVLYYSEDTHYSVYKNLRLLNMRHIMIKSQPNGEIDYGDLYETMRIHRDCPPIIFANIGTTMTEGIDNVRTIQDMLKKLAIHESHIHCDAALAGMTLPFMDDAPAFDFATGIDSISISGHKFIGSPIPCGIVLTKKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 297 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaattcgaacgcgaggttctggcgtggttcgccgaactcatgcacattgcaccagaagaatactggggttatgtgactaatggaggaaccgagggcaacctgtacggtttgtacttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgagggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgatcagcatctccggtcacaaattcattgggtctccgattccttgcggcatcgttttgacacgtaaacattatgtggagcgtattgctcgcagtatcgaatatgtcggtaccctggacactacgattaccggcagccgtaatggtatcacaccgctgtttctttggtacgcggtgaaacaagctggacgcgatggcttcaaacgtcgtgttgcggcatgtctggataacgcccgctatacggtcgagcagtttaaacgcatcggtgtagaagcgtggcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 298 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREFEREVLAWFAELMHIAPEEYWGYVTNGGTEGNLYGLYLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSISISGHKFIGSPIPCGIVLTRKHYVERIARSIEYVGTLDTTITGSRNGITPLFLWYAVKQAGRDGFKRRVAACLDNARYTVEQFKRIGVEAWRNPCAITVVFPKPPQSVLHKWQIAVNGDRAHIILMPSTTLQHVDELIADIAAANKAAAAPSDTQKPAAKKRPTPAGAPQRTT

SEQ ID NO: 299 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaattcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcctgtactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgagggcattgacaacgtacgcaccatccaggatatgctgaagaaactggcgattcacgaatctcatatccattgtgacgcagcattagcggggatgaccctgccctttatggatgatgccccagcgtttgactttgccactggcattgattcgatcagcatctccggtcacaaattcattgggtctccgattccttgcggcatcgttttgacacgtaaacattatgtggagcgtattgctcgcagtatcgaatatgtcggtaccctggacactacgattaccggcagccgtaatggtatcacaccgctgtttctttggtacgcggtgaaacaagctggacgcgatggcttcaaacgtcgtgttgcggcatgtctggataacgcccgctatacggtcgagcagtttaaacgcatcggtgtagaagcgtggcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 300 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREFEREVLAWFAELMHIAPEEYWGYVTNGGTEGNLYGLFLARERFPTGVLYYSEDTHYSVYKNLRLLNMRHIMIKSQPNGEIDYGDLYETMRIHRDCPPIIFANIGTTMTEGIDNVRTIQDMLKKLAIHESHIHCDAALAGMTLPFMDDAPAFDFATGIDSISISGHKFIGSPIPCGIVLTRKHYVERIARSIEYVGTLDTTITGSRNGITPLFLWYAVKQAGRDGFKRRVAACLDNARYTVEQFKRIGVEAWRNPCAITVVFPKPPQSVLHKWQIAVNGDRAHIILMPSTTLQHVDELIADIAAANKAAAAPSDTQKPAAKKRPTPAGAPQRTT

SEQ ID NO: 301 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaattcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgctgaagaaactggcgattcacgaatctcatatccattgtgacgcagcattagcggggatgaccctgccctttatggatgatgccccagcgtttgactttgccactggcattgattcgatcagcatctccggtcacaaattcattgggtctccgattccttgcggcatcgttttgacacgtaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 302 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREFEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMLKKLAIHESHIHCDAALAGMTLPFMDDAPAFDFATGIDSISISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 303 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttctgtaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 304 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFCNIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 305 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatgctattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 306 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNAITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 307 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaattcgaacgcgaggttctggcgtggttcgccgaactcatgcacattgcaccagaagaatactggggttatgtgactaatggaggaaccgagggcaacctgtacggtttgtacttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgctgagcatctccggtcacaaattcattgggtctccgattccttgcggcatcgttttgacacgcaaacattatgtggagcgtattgctcgcagtatcgaatatgtcggtaccctggacactacgattaccggcagccgtaatggtatcacaccgctgtttctttggtacgcggtgaaacaagctggacgcgatggcttcaaacgtcgtgttgcggcatgtctggataacgcccgctatacggtcgagcagtttaaacgcatcggtgtagaagcgtggcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 308 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREFEREVLAWFAELMHIAPEEYWGYVTNGGTEGNLYGLYLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEAWRNPCAITVVFPKPPQSVLHKWQIAVNGDRAHIILMPSTTLQHVDELIADIAAANKAAAAPSDTQKPAAKKRPTPAGAPQRTT

SEQ ID NO: 309 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaattcgaacgcgaggttctggcgtggttcgccgaactcatgcacattgcaccagaagaatactggggttatgtgactaatggaggaaccgagggcaacctgtacggtttgtacttagcgcgtgaacgctttcctacgggcgtcctgtactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgctgaagaaactggcgattcacgaatctcatatccattgtgacgcagcattagcggggatgaccctgccctttatggatgatgccccagcgtttgactttgccactggcattgattcgctgagcatctccggtcacaaattcattgggtctccgattccttgcggcatcgttttgacacgtaaacattatgtggagcgtattgctcgcagtatcgaatatgtcggtaccctggacactacgattaccggcagccgtaatggtatcacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 310 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREFEREVLAWFAELMHIAPEEYWGYVTNGGTEGNLYGLYLARERFPTGVLYYSEDTHYSVYKNLRLLNMRHIMIKSQPNGEIDYGDLYETMRIHRDCPPIIFANIGTTMTEGIDNVRTIQDMLKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 311 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaattcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgagggcattgacaacgtacgcaccatccaggatatgctgaagaaactggcgattcacgaatctcatatccattgtgacgcagcattagcggggatgaccctgccctttatggatgatgccccagcgtttgactttgccactggcattgattcgctgagcatctccggtcacaaattcattgggtctccgattccttgcggcatcgttttgacaaagaaacattatgtggagcgtattgctcgcagtatcgaatatgtcggtaccctggacactacgattaccggcagccgtaatggtatcacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 312 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREFEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMLKKLAIHESHIHCDAALAGMTLPFMDDAPAFDFATGIDSLSISGHKFIGSPIPCGIVLTKKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 313 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtgtctcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 314 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVSRIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 315 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccatcaacaacgtaggcgatccgtttgctgcatccagttatcgcgtgcatacccgtgaattcgaacgcgaggttctggcgtggttcgccgaactcatgcacattgcaccagaagaatactggggttatgtgactaatggaggaaccgagggcaacctgtacggtttgtacttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgagggcattgacaacgtacgcaccatccaggatatgctgaagaaactggcgattcacgaatctcatatccattgtgacgcagcattagcggggatgaccctgccctttatggatgatgccccagcgtttgactttgccactggcattgattcgatcagcatctccggtcacaaattcattgggtctccgattccttgcggcatcgttttgacacgcaaacattatgtggagcgtattgctcgcagtatcgaatatgtcggtaccctggacactacgattaccggcagccgtaatggtcacacaccgctgtttctttggtacgcggtgaaacaagctggacgcgatggcttcaaacgtcgtgttgcggcatgtctggataacgcccgctatacggtcgagcagtttaaacgcatcggtgtagaagcgtggcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 316 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPINNVGDPFAASSYRVHTREFEREVLAWFAELMHIAPEEYWGYVTNGGTEGNLYGLYLARERFPTGVAYYSEDTHYSVYKNLRLLNMRHIMIKSQPNGEIDYGDLYETMRIHRDCPPIIFANIGTTMTEGIDNVRTIQDMLKKLAIHESHIHCDAALAGMTLPFMDDAPAFDFATGIDSISISGHKFIGSPIPCGIVLTRKHYVERIARSIEYVGTLDTTITGSRNGHTPLFLWYAVKQAGRDGFKRRVAACLDNARYTVEQFKRIGVEAWRNPCAITVVFPKPPQSVLHKWQIAVNGDRAHIILMPSTTLQHVDELIADIAAANKAAAAPSDTQKPAAKKRPTPAGAPQRTT

SEQ ID NO: 317 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccatcaacaacgtaggcgatccgtttgctgcatccagttatcgcgtgcatacccgtgaattcgaacgcgaggttctggcgtggttcgccgaactcatgcacattgcaccagaagaatactggggttatgtgactaatggaggaaccgagggcaacctgtacggtttgtacttagcgcgtgaacgctttcctacgggcgtcctgtactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgagggcattgacaacgtacgcaccatccaggatatgctgaagaaactggcgattcacgaatctcatatccattgtgacgcagcattagcggggatgaccctgccctttatggatgatgccccagcgtttgactttgccactggcattgattcgatcagcatctccggtcacaaattcattgggtctccgattccttgcggcatcgttttgacaaagaaacattatgtggagcgtattgctcgcagtatcgaatatgtcggtaccctggacactacgattaccggcagccgtaatggtatcacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 318 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPINNVGDPFAASSYRVHTREFEREVLAWFAELMHIAPEEYWGYVTNGGTEGNLYGLYLARERFPTGVLYYSEDTHYSVYKNLRLLNMRHIMIKSQPNGEIDYGDLYETMRIHRDCPPIIFANIGTTMTEGIDNVRTIQDMLKKLAIHESHIHCDAALAGMTLPFMDDAPAFDFATGIDSISISGHKFIGSPIPCGIVLTKKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 319 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaattcgaacgcgaggttctggcgtggttcgccgaactcatgcacattgcaccagaagaatactggggttatgtgactaatggaggaaccgagggcaacctgtacggtttgttcttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgagggcattgacaacgtacgcaccatccaggatatgctgaagaaactggcgattcacgaatctcatatccattgtgacgcagcattagcggggatgaccctgccctttatggatgatgccccagcgtttgactttgccactggcattgattcgctgagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 320 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREFEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMLKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 321 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaaatttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 322 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLNFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 323 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccaattatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaaagccatatccattgtgacgcagcattagcggggatgaccctgccctttatggatgatgccccagcgtttgactttgccactggcattgattcgcttagcatctccggtcacaaattcattgggtctccgattccttgcggcatcgttttgactcgcaaacattatgtggagcgtattgctcgcagtatcgaatatgtcggtaccctggacactacgattaccggcagccgtaatggtattacaccgctgtttctttggtacgcgatcaaacaagctggacgcgatggcttcaaacgtcgtgttgcggcatgtctggataacgcccgctatacggtcgagcagtttaaacgcatcggtgtagaagcgcgtcgtaatccatgctcgattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 324 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASNYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAIKQAGRDGFKRRVAACLDNARYTVEQFKRIGVEARRNPCSITVVFPKPPQSVLHKWQIAVNGDRAHIILMPSTTLQHVDELIADIAAANKAAAAPSDTQKPAAKKRPTPAGAPQRTT

SEQ ID NO: 325 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccatcaacaacgtaggcgatccgtttgctgcatccagttatcgcgtgcatacccgtgaattcgaacgcgaggttctggcgtggttcgccgaactcatgcacattgcaccagaagaatactggggttatgtgactaatggaggaaccgagggcaacctgtacggtttgtacttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattatcggggatgaccctgccctttatggatgatgccccagcgtttgactttgccactggcattgattcgctgagcatctccggtcacaaattcattgggtctccgattccttgcggcatcgttttgacacgtaaacattatgtggagcgtattgctcgcagtatcgaatatgtcggtaccctggacactacgattaccggcagccgtaatggtatcacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 326 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPINNVGDPFAASSYRVHTREFEREVLAWFAELMHIAPEEYWGYVTNGGTEGNLYGLYLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALSGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 327 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccaattatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatatcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcgatcaaacaagctggacgcgatggcttcaaacgtcgtgttgcggcatgtctggataacgcccgctatacggtcgagcagtttaaacgcatcggtgtagaagcgcgtcgtaatccatgctcgattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 328 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASNYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHEYHIHCDAALAGMTLPFMDDAPAFDFATGIDSLSISGHKFIGSPIPCGIVLTRKHYVERIARSIEYVGTLDTTITGSRNGITPLFLWYAIKQAGRDGFKRRVAACLDNARYTVEQFKRIGVEARRNPCSITVVFPKPPQSVLHKWQIAVNGDRAHIILMPSTTLQHVDELIADIAAANKAAAAPSDTQKPAAKKRPTPAGAPQRTT

SEQ ID NO: 329 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctgaagtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 330 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLKWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 331 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttacggcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacactggctggtgcaccgcaacgtactacg

SEQ ID NO: 332 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFTASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTLAGAPQRTT

SEQ ID NO: 333 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccaattatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgttcgcaccatccaggatatgttcaagaaactggggattcacgaatatcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcgatcaaacaagctggacgcgatggcttcaaacgtcgtgttgcggcatgtctggataacgcccgctatacggtcgagcagctgaaacgcatcggtgtagaagcgcgtcgtaatccatgctcgattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 334 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASNYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLGIHEYHIHCDAALAGMTLPFMDDAPAFDFATGIDSLSISGHKFIGSPIPCGIVLTRKHYVERIARSIEYVGTLDTTITGSRNGITPLFLWYAIKQAGRDGFKRRVAACLDNARYTVEQLKRIGVEARRNPCSITVVFPKPPQSVLHKWQIAVNGDRAHIILMPSTTLQHVDELIADIAAANKAAAAPSDTQKPAAKKRPTPAGAPQRTT

SEQ ID NO: 335 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccggagccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 336 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPEPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 337 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgggggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 338 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTGGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 339 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttgagggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 340 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVEGDRAHIILMPSTTLQHVDELIADIAAANKAAAAPSDTQKPAAKKRPTPAGAPQRTT

SEQ ID NO: 341 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactgagtattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 342 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLSIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 343 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcactcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 344 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELTHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 345 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgttttcctacgggcgtcgcctactattcggaagacacccattatagcgtgtataagaatctgcgtctgctgaacatgcgtcacatcatgatcaaatcacagccgaatggtgagattgattacggcgatctctacgaaactattcgcattcatcgtgattgcccgccgatcatcttcgctaacattggtacgacgatgaccgaaggcattgacaacattcgcaccatccaggatatgttcaagaaactggcgattcacgaatatcatatccattgtgacgcagcattagcggggatgaccctgccctttatggatgatgccccagcgtttgactttgccactggcattgattcgcttagcgtttccggtcacaaattcattgggtctccgattccttgcggcatcgttttggctcgcaaacattatgtggagcgtattgctcgcagtatcgaatatgtcggtaccctggacactacgattaccggcagccgtaatggtattacaccgctgtttctttggtacgcgatcaaacaagctggacgcgatggcttcaaacgtcgtgttgcggcatgtctggataacgcccgctatacggtcgagcagctgaaacgcatcggtgtagaagcgcgtcgtaatccatgcgcgattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 346 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETIRIHRDCPPIIFANIGTTMTEGIDNIRTIQDMFKKLAIHEYHIHCDAALAGMTLPFMDDAPAFDFATGIDSLSVSGHKFIGSPIPCGIVLARKHYVERIARSIEYVGTLDTTITGSRNGITPLFLWYAIKQAGRDGFKRRVAACLDNARYTVEQLKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 347 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaagtgattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 348 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEVIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 349 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatattcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 350 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNIRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 351 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccatcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgttcttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgagggcattgacaacgtacgcaccatccaggatatgctgaagaaactggcgattcacgaatctcatatccattgtgacgcagcattatcggggatgaccctgccctttatggatgatgccccagcgtttgactttgccactggcattgattcgatcagcatctccggtcacaaattcattgggtctccgattccttgcggcatcgttttgacaaagaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 352 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPINNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMLKKLAIHESHIHCDAALSGMTLPFMDDAPAFDFATGIDSISISGHKFIGSPIPCGIVLTKKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 353 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccatcaacaacgtaggcgatccgtttgctgcatccagttatcgcgtgcatacccgtgaattcgaacgcgaggttctggcgtggttcgccgaactcatgcacattgcaccagaagaatactggggttatgtgactaatggaggaaccgagggcaacctgtacggtttgttcttagcgcgtgaacgctttcctacgggcgtcctgtactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgatcagcatctccggtcacaaattcattgggtctccgattccttgcggcatcgttttgacaaagaaacattatgtggagcgtattgctcgcagtatcgaatatgtcggtaccctggacactacgattaccggcagccgtaatggtatcacaccgctgtttctttggtacgcggtgaaacaagctggacgcgatggcttcaaacgtcgtgttgcggcatgtctggataacgcccgctatacggtcgagcagtttaaacgcatcggtgtagaagcgtggcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 354 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPINNVGDPFAASSYRVHTREFEREVLAWFAELMHIAPEEYWGYVTNGGTEGNLYGLFLARERFPTGVLYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSISISGHKFIGSPIPCGIVLTKKHYVERIARSIEYVGTLDTTITGSRNGITPLFLWYAVKQAGRDGFKRRVAACLDNARYTVEQFKRIGVEAWRNPCAITVVFPKPPQSVLHKWQIAVNGDRAHIILMPSTTLQHVDELIADIAAANKAAAAPSDTQKPAAKKRPTPAGAPQRTT

SEQ ID NO: 355 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccatcaacaacgtaggcgatccgtttgctgcatccagttatcgcgtgcatacccgtgaattcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgctgaagaaactggcgattcacgaatctcatatccattgtgacgcagcattagcggggatgaccctgccctttatggatgatgccccagcgtttgactttgccactggcattgattcgatcagcatctccggtcacaaattcattgggtctccgattccttgcggcatcgttttgacacgcaaacattatgtggagcgtattgctcgcagtatcgaatatgtcggtaccctggacactacgattaccggcagccgtaatggtatcacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 356 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPINNVGDPFAASSYRVHTREFEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMLKKLAIHESHIHCDAALAGMTLPFMDDAPAFDFATGIDSISISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 357 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccatcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgagggcattgacaacgtacgcaccatccaggatatgttcaagaaactggcgattcacgaatctcatatccattgtgacgcagcattatcggggatgaccctgccctttatggatgatgccccagcgtttgactttgccactggcattgattcgcttagcatctccggtcacaaattcattgggtctccgattccttgcggcatcgttttgacaaagaaacattatgtggagcgtattgctcgcagtatcgaatatgtcggtaccctggacactacgattaccggcagccgtaatggtatcacaccgctgtttctttggtacgcggtgaaacaagctggacgcgatggcttcaaacgtcgtgttgcggcatgtctggataacgcccgctatacggtcgagcagtttaaacgcatcggtgtagaagcgtggcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 358 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPINNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALSGMTLPFMDDAPAFDFATGIDSLSISGHKFIGSPIPCGIVLTKKHYVERIARSIEYVGTLDTTITGSRNGITPLFLWYAVKQAGRDGFKRRVAACLDNARYTVEQFKRIGVEAWRNPCAITVVFPKPPQSVLHKWQIAVNGDRAHIILMPSTTLQHVDELIADIAAANKAAAAPSDTQKPAAKKRPTPAGAPQRTT

SEQ ID NO: 359 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcataccgaggaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 360 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTEEIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 361 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttgttccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 362 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLVPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 363 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcatacccgtgaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactcttcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 364 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTREIEREVLAWFAELMHIAPEEYWGYVTNGGTEGNLYGLFLARERFPTGVAYYSEDTHYSVYKNLRLLNMRHIMIKSQPNGEIDYGDLYETLRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT

SEQ ID NO: 365 - DNA, 1218 bp, synthetic construct
  misc_feature 1..1218: note = Amino Acid Decarboxylase from Planctomycetaceae bacterium
  source 1..1218: mol_type = other DNA
atgggtaatctgaaatttccagatggcgcgttgagcccgcatgaccgccgccgtctggaagatgtgtatcagtacgttcgtgagcaatgcgacaccttcattgggtatccgtgtacgatgcactttgattacacacctcttttaccgttcctggcctatcccgtcaacaacgtaggcgatccgtttgctgcatccagttatcgcgtgcataccgcggaaatcgaacgcgaggttctggcgtggttcgccgaactcatgcacattgcaccagaagaatactggggttatgtgactaatggaggaaccgagggcaacctgtacggtttgtttttagcgcgtgaacgctttcctacgggcgtcgcctactattcggaagacacccattatagcgtgtataagaatctgcgtctgctgaacatgcgtcacatcatgatcaaatcacagccgaatggtgagattgattacggcgatctctacgaaactatgcgcattcatcgtgattgcccgccgatcatcttcgctaacattggtacgacgatgaccgaaggcattgacaacgtacgcaccatccaggatatgttcaagaaactggcgattcacgaatctcatatccattgtgacgcagcattagcggggatgaccctgccctttatggatgatgccccagcgtttgactttgccactggcattgattcgcttagcatctccggtcacaaattcattgggtctccgattccttgcggcatcgttttgacacgcaaacattatgtggagcgtattgctcgcagtatcgaatatgtcggtaccctggacactacgattaccggcagccgtaatggtattacaccgctgtttctttggtacgcggtgaaacaagctggacgcgatggcttcaaacgtcgtgttgcggcatgtctggataacgcccgctatacggtcgagcagtttaaacgcatcggtgtagaagcgcgtcgtaatccatgcgccattaccgttgtgttcccgaaacctccgcagtcagtgctgcacaagtggcaaatcgcagttaacggcgaccgcgcgcatattattttaatgccaagtaccaccctccagcatgtcgatgaattgatcgctgacattgcagccgccaataaagcggctgcagcaccgagcgatacgcagaaacccgccgccaagaaacgcccgacaccggctggtgcaccgcaacgtactacg

SEQ ID NO: 366 - protein, 406 aa, synthetic construct
  REGION 1..406: note = Amino Acid Decarboxylase from Planctomycetaceae bacterium
MGNLKFPDGALSPHDRRRLEDVYQYVREQCDTFIGYPCTMHFDYTPLLPFLAYPVNNVGDPFAASSYRVHTAEIEREVLAWFAELMHIAPEEYWGYVTNGGTEGNLYGLFLARERFPTGVAYYSEDTHYSVYKNLRLLNMRHIMIKSQPNGEIDYGDLYETMRIHRDCPPIIFANIGTTMTEGIDNVRTIQDMFKKLAIHESHIHCDAALAGMTLPFMDDAPAFDFATGIDSLSISGHKFIGSPIPCGIVLTRKHYVERIARSIEYVGTLDTTITGSRNGITPLFLWYAVKQAGRDGFKRRVAACLDNARYTVEQFKRIGVEARRNPCAITVVFPKPPQSVLHKWQIAVNGDRAHIILMPSTTLQHVDELIADIAAANKAAAAPSDTQKPAAKKRPTPAGAPQRTT